(19) United States
(12) Patent Application Publication
(10) Pub. No.: US 2025/0281466 A1
(43) Pub. Date: Sep. 11, 2025

(54) USE OF CLOPIDOGREL OR COMBINATIONS THEREOF TO TREAT AMYOTROPHIC LATERAL SCLEROSIS, DAMAGES CAUSED BY THE NORMAL AGING PROCESS, NERVE INJURIES, MUSCULAR DYSTROPHY, STRENUOUS PHYSICAL ACTIVITIES AND OTHER CONDITIONS THAT AFFECT SYNAPSES AS WELL AS SKELETAL MUSCLES

(71) Applicant: Brown University, Providence, RI (US)

(72) Inventor: Gregorio Valdez, Barrington, RI (US)

(21) Appl. No.: 19/072,783

(22) Filed: Mar. 6, 2025

Related U.S. Application Data
(60) Provisional application No. 63/562,661, filed on Mar. 7, 2024.

(51) Int. Cl. A61K 31/4365; A61K 31/7076; A61K 45/06; A61P 25/28

(52) U.S. Cl. CPC A61K 31/4365; A61K 31/7076; A61K 45/06; A61P 25/28

(57) ABSTRACT

New treatments using Clopidogrel, Ticagrelor, Prasugrel, and/or Cangrelor and new uses for Clopidogrel are disclosed herein. For example, it has been found that conditions such as ALS (Amyotrophic Lateral Sclerosis), Alzheimer's Disease, the normal aging process, nerve damages, nerve injuries, muscular dystrophy, strenuous physical activities, fatigue, and other conditions that affect synapses as well as skeletal muscles can be treated using Clopidogrel or pharmaceutically acceptable other forms. New mechanisms and modes of action are explained herein.

### CROSS REFERENCE TO RELATED APPLICATIONS

This application claims the benefit of priority to U.S. Provisional Patent No. 63/562,661, filed 7 Mar. 2024, the entire disclosure of which is incorporated by reference as if fully set forth herein in its entirety.

### FIELD OF THE INVENTION

The embodiments of the present invention relate to novel mechanisms of and novel uses for Clopidogrel, which can be sold under the trade name Plavix®, either alone or when applied in combination therapies with one or more of Ticagrelor, Prasugrel, and/or Cangrelor optionally with any other therapeutic agent/treatment.

### FIELD STATEMENT REGARDING FEDERALLY SPONSORED RESEARCH OR DEVELOPMENT

Not applicable (N/A).

### BACKGROUND OF THE INVENTION

Clopidogrel, which can be known by the trade name Plavix®, is a generic drug that can be used to treat cardiovascular conditions. Clopidogrel is sold by several drug makers. Clopidogrel is an FDA-approved drug with few recognized side effects. Clopidogrel can be used to prevent heart attack and stroke in people who are at high risk of these events, including those with a history of myocardial infarction and other forms of acute coronary syndrome, stroke, and those with peripheral artery disease.

The histories of each of Clopidogrel, Ticagrelor, Prasugrel, and Cangrelor are deeply rooted in the development of antiplatelet therapies, which have been pivotal in the management of cardiovascular diseases in humans. For example, Clopidogrel, a thienopyridine class antiplatelet agent, was first approved in the late 1990s. It works by inhibiting a receptor on platelets, thereby preventing platelet aggregation. This mechanism of action has made Clopidogrel a cornerstone in the prevention of thrombotic events in patients with coronary artery disease, peripheral vascular disease, and cerebrovascular disease.

Ticagrelor, introduced in the early 2010s, is a cyclopentyltriazolopyrimidine that also targets platelets but does so in a reversible manner. Unlike Clopidogrel, Ticagrelor does not require metabolic activation, which allows for a more rapid onset of action. This characteristic has made Ticagrelor a preferred choice in acute coronary syndrome settings, where quick platelet inhibition is crucial.

Prasugrel, another thienopyridine, was developed to overcome some of the limitations associated with Clopidogrel, such as variable patient response due to genetic polymorphisms in platelets and also affecting drug metabolism. Approved in the late 2000s, Prasugrel provides more consistent platelet inhibition and has been shown to be particularly effective in patients undergoing percutaneous coronary intervention.

Cangrelor, a non-thienopyridine targeting platelets, is unique in its intravenous administration and rapid onset and offset of action on platelets. Approved in the mid-2010s, Cangrelor is used in situations where oral antiplatelet therapy is not feasible, such as during percutaneous coronary interventions when immediate platelet inhibition is required.

Amyotrophic lateral sclerosis (ALS) is also known as motor neurone disease (MND) or Lou Gehrig's disease. ALS is a tragic, rare, and terminal neurodegenerative disease that results in the progressive loss of motor neurons that control voluntary muscles. Examples of early symptoms of ALS can include stiff muscles, muscle twitches, gradual increasing weakness, and muscle wasting. Motor neuron loss continues until the abilities to eat, speak, move, or, lastly, breathe are lost. Both genetic and environmental factors are thought to be involved in ALS. Unfortunately, there is no cure for ALS, and treatments do not currently provide much hope for families. In addition to ALS, there are many neuromuscular conditions and neuronal conditions that have no effective treatments. What is urgently needed are new treatments for ALS and for other conditions that cause instability and degeneration of muscle and other synapses.

### BRIEF SUMMARY OF THE INVENTION

The following presents a simplified summary of the innovation, or technology herein, in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

Neuromuscular and nervous conditions encompass a wide range of disorders that affect the muscles and the nerves that control them. These conditions can lead to significant impairment in movement, coordination, and overall quality of life. Common neuromuscular disorders include muscular dystrophy, amyotrophic lateral sclerosis (ALS), and myasthenia gravis, among others. These disorders often result in the degeneration of neuromuscular junctions (NMJs), which are critical for the transmission of signals from nerves to muscles. The degeneration of NMJs can lead to muscle weakness, fatigue, and atrophy, posing a substantial challenge for affected individuals and healthcare providers.

Current treatment options for neuromuscular conditions are limited and primarily focus on managing symptoms rather than addressing the underlying causes of NMJ degeneration. There is a pressing need for therapeutic strategies that can preserve or repair NMJs to improve patient outcomes. The development of such treatments is crucial, as they hold the potential to not only alleviate symptoms but also to slow or halt the progression of these debilitating conditions. As research in this area continues to evolve, innovative approaches that target the preservation and repair of NMJs are disclosed herein to meet the unmet medical needs of patients with neuromuscular disorders.

In some embodiments, a method of treating a neuromuscular condition or a nervous condition in a subject in need thereof is disclosed herein, the method comprising the steps of: (a) obtaining a subject diagnosed with a condition, suspected of having the condition, or a subject with a likelihood of developing the condition; and (b) administering a therapeutically effective amount of Clopidogrel (formula I):

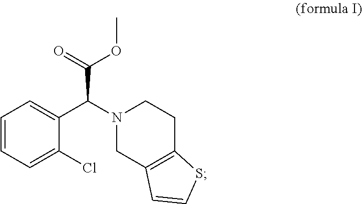 [SMILES: COC(=O)[C@H](c1ccccc1Cl)N1CCc2sccc2C1]

or of administering one or more of Clopidogrel, Ticagrelor, Prasugrel, and/or Cangrelor, a therapeutically effective amount of a salt of the one or more thereof, a prodrug of the one or more thereof, a hydrate and/or a solvate of the one or more thereof, or a combination thereof.

According to some aspects, the method is wherein the one or more of Clopidogrel, Ticagrelor, Prasugrel, and/or Cangrelor is included within a pharmaceutical formulation.

In some embodiments, the method above can be wherein the condition comprises ALS (Amyotrophic Lateral Sclerosis), Alzheimer's Disease, the normal aging process, nerve damages, nerve injuries, muscular dystrophy, strenuous physical activities, fatigue, and other conditions that affect synapses as well as skeletal muscles.

According to some aspects, the method is wherein the method modulates P2Y12 receptors that are found in perisynaptic Schwann cells in the subject.

In some embodiments, the method is executed further comprising administering an additional therapeutic agent either before, during, or after the administration of a therapeutically effective amount of Clopidogrel (formula I), Ticagrelor, Prasugrel, and/or Cangrelor or a pharmaceutically effective amount of a salt thereof, a prodrug thereof, a hydrate and/or solvate thereof, or a combination thereof.

According to some aspects, the method is wherein formula I further comprises a pharmaceutically acceptable anion, in a non-limiting example of such:

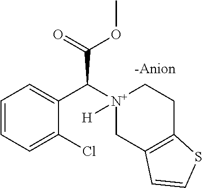

(formula II, an example of a salt of formula I); which can represent salt of Clopidogrel, Ticagrelor, Prasugrel, and/or Cangrelor with Anion and/or Cation.

In some embodiments, the salt shown above can be wherein the -Anion includes bisulfate, chloride, bromide, carbide, fluoride, hydride, iodide, nitride, phosphide, oxide, sulfide, selenide, azide, peroxide, triiodide, carbonate, chlorate, chromate, dichromate, dihydrogen phosphate, hydrogen carbonate, hydrogen sulfate, hydrogen sulfite, hydroxide, hypochlorite, mono-hydrogen phosphate, nitrate, nitrite, perchlorate, permanganate, peroxide, phosphate, sulfate, sulfite, superoxide, thiosulfate, silicate, metasilicate, aluminum silicate, acetate, formate, oxalate, a combination thereof, or any of the other anions disclosed in the sections below.

According to some aspects, the methods herein can be wherein the formula I or formula II, or Clopidogrel, Ticagrelor, Prasugrel, and/or Cangrelor further comprises a hydrate or a solvate made by evaporating a slurry or a solution of formula I in water or as solvent until hydrogen bonding occurs between the water and/or solvent and formula I, and a solid hydrate and/or solvate results. In this example, a formation of the hydrate or the solvate can usually be confirmed by an ATR-IR (attenuated total reflection)-IR of the hydrate or solvate solid having an —OH stretch region, as compared to the non-hydrate or non-solvate solid which does not exhibit an —OH stretch region in the ATR-IR spectrum.

In some embodiments, the methods of any embodiment disclosed herein can be used in combination with a physical therapy, a chemotherapy, or an electromagnetic radiation or radiation therapy.

The invention contemplates that the above-described compounds or combinations of one or more can be derivatized or can be structurally altered, for example, by addition or substitution of one or more atoms using a radioisotope or using a different element (e.g., B or boron in place of C or carbon), by removal of an ester or by addition of a salt form, an amino acid, a sugar, or a peptide. Hydrates and/or solvates can be formed by 1) dissolving the molecule in water and/or solvent and slowly drying, whereby water and/or solvent remain hydrogen bonded with OH groups in the molecule or associated with the molecule. A formation of a hydrate or solvate can typically be confirmed by an attenuated total reflection (ATR) infrared spectrum acquired from the solid-state sample. The ATR spectrum of a hydrate or solvate will typically show increased broad bands (indicating hydrogen bonding) above about 3200 cm⁻¹, as compared to the non-hydrate or non-solvate solid sample. In some embodiments, the above-described compounds are attached to or associated with a targeting moiety. In some embodiments, the targeting moiety is a particle or an antibody with affinity for a specific type of cell.

In some embodiments, the technology provides a method of treating a neuromuscular condition or a nervous condition in a subject in need thereof, the method comprising the steps of: (a) obtaining a subject diagnosed with a condition, suspected of having the condition, or a subject with a likelihood of developing the condition; and (b) administering a therapeutically effective amount of one or more of Clopidogrel, Ticagrelor, Prasugrel, and/or Cangrelor or a therapeutically effective amount of a salt of Clopidogrel, Ticagrelor, Prasugrel, and/or Cangrelor thereof, a prodrug of Clopidogrel, Ticagrelor, Prasugrel, and/or Cangrelor thereof, a hydrate and/or a solvate of Clopidogrel, Ticagrelor, Prasugrel, and/or Cangrelor thereof, or a combination thereof; whereby neuromuscular junctions (NMJs) are preserved and/or repaired in the subject by the administering or whereby a number of denervated neuromuscular junctions (NMJs) in the subject are reduced by the administering.

New methods are needed for preserving and/or repairing neuromuscular junctions (NMJs) in humans; the technology disclosed herein are new methods for Clopidogrel, Ticagrelor, Prasugrel, and/or Cangrelor, which are all FDA-approved drugs that work by blocking the activity of a specific purinergic receptor called P2Y12. The technology herein has newly discovered P2Y12 expressed in a special type of Schwann cells, called perisynaptic Schwann cells. These cells are critical for the maintenance and repair of the synapse critical for the viability and function of skeletal muscles. We have gathered data showing that muscles maintain their synapses in a mouse model of Amyotrophic Lateral Sclerosis (ALS) treated with Clopidogrel, Ticagrelor, Prasugrel, and/or Cangrelor but not vehicle. This finding indicate that Clopidogrel, Ticagrelor, Prasugrel, and/or Cangrelor can be used to treat ALS and other conditions that cause instability and degeneration of muscle and other synapses. The technology contemplates tautomers, isomers, and pro-drugs of the small molecules used herein. In some embodiments, perisynaptic Schwann cells (PSCs) are glial cells that wrap around nerve terminals at the neuromuscular junction (NMJ). They play a role in synaptic transmission, nerve regeneration, and synapse development.

As an additional brief summary, for example, to provide discussion points for a brief summary, some features of the technology disclosed herein can be briefly summarized (or discussed) by the following list of features, any of which can be inter-combined with any other feature, detail, embodiment, discussion point, aspect, or example disclosed herein:
Feature 1: A method of treating a neuromuscular condition or a nervous condition in a subject in need thereof, the method comprising the steps of: (1) obtaining a subject diagnosed with a condition, suspected of having the condition, or a subject with a likelihood of developing the condition; and (2) administering a therapeutically effective amount of one or more of Clopidogrel, Ticagrelor, Prasugrel, and/or Cangrelor, a therapeutically effective amount of a salt of the one or more thereof, a prodrug of the one or more thereof, a hydrate and/or a solvate of the one or more thereof, or a combination thereof; whereby neuromuscular junctions (NMJs) are preserved and/or repaired in the subject by the administering.
Feature 2: The method of feature 1, wherein the neuromuscular condition is selected from the group consisting of amyotrophic lateral sclerosis (ALS), spinal muscular atrophy (SMA), Duchenne muscular dystrophy, Becker muscular dystrophy, myasthenia gravis, Charcot-Marie-Tooth disease, spinal cord injury, a condition due to the normal aging process, sarcopenia, cachexia, peripheral demyelination diseases, a condition due to pregnancy, a condition due to menopause, nerve damages, nerve injuries, damages due to strenuous physical activities, fatigue, or other conditions that affect synapses and/or skeletal muscles
Feature 3: The method of feature 1, wherein the nervous condition is selected from the group consisting of Alzheimer's disease, Parkinson's disease, Huntington's disease, multiple sclerosis, amyotrophic lateral sclerosis (ALS), spinal muscular atrophy (SMA), Charcot-Marie-Tooth disease, Friedreich's ataxia, Lewy body dementia, vascular dementia, frontotemporal dementia, progressive supranuclear palsy, corticobasal degeneration, chronic traumatic encephalopathy, Creutzfeldt-Jakob disease, and/or neurodegenerative disorders.
Feature 4: The method of feature 1, wherein the administering is performed via a route selected from the group consisting of oral, parenteral, subcutaneous, intravenous, intramuscular, intraperitoneal, transdermal, intranasal, and rectal administration.
Feature 5: The method of feature 1, wherein the Clopidogrel, Ticagrelor, Prasugrel, and/or Cangrelor or a pharmaceutically acceptable form is administered in a dosage form optimized for the selected route of administration, the dosage form being selected from the group consisting of a tablet, a capsule, a solution, a suspension, an emulsion, a powder, a granule, a suppository, an injection, an infusion, and an implant.
Feature 6: The method of feature 1, wherein the therapeutically effective amount of Clopidogrel, Ticagrelor, Prasugrel, and/or Cangrelor is determined based on the severity and progression of the neuromuscular or nervous condition, an age, weight, and general health of the subject, and an optimal balance between therapeutic efficacy and potential side effects, and ranges from about 1 mg to about 1000 mg per day.
Feature 7: The method of feature 1, wherein the therapeutically effective amount of Clopidogrel, Ticagrelor, Prasugrel, and/or Cangrelor is administered as a single daily dose or divided into multiple doses per day to maintain a consistent plasma concentration and to minimize potential adverse effects while maximizing therapeutic benefits.
Feature 8: The method of feature 1, wherein the therapeutically effective amount of Clopidogrel, Ticagrelor, Prasugrel, and/or Cangrelor is administered in a designed dosing regimen of once, twice, or thrice daily, taking into account the pharmacokinetic and pharmacodynamic properties of Clopidogrel, Ticagrelor, Prasugrel, and/or Cangrelor, to ensure optimal receptor occupancy and to minimize the risk of tachyphylaxis or tolerance development.
Feature 9: The method of feature 1, wherein the method modulates P2Y12 receptors, P2Y1 receptors, P2X receptors, adenosine receptors, 5-HT receptors, NMDA receptors, AMPA receptors, kainate receptors, or a combination thereof that are found in perisynaptic Schwann cells in the subject.
Feature 10: The method of feature 1, wherein the method is executed in combination with a physical therapy, a chemotherapy, an electromagnetic radiation or radiation therapy, a gene therapy, a stem cell therapy, administration with other drugs, or a combination thereof.
Feature 11: A method of preserving and repairing damages to neuromuscular junctions (NMJs) in a subject having, suspected of having, or likely to develop a neuromuscular condition or a nervous condition, the method comprising: administering to the subject a therapeutically effective amount of Clopidogrel, Ticagrelor, Prasugrel, and/or Cangrelor and/or one or more pharmaceutically acceptable forms thereof.
Feature 12: The method of feature 11, wherein the subject is diagnosed with the neuromuscular condition or the nervous condition, is suspected of having the neuromuscular condition or the nervous condition, or has a likelihood of developing the neuromuscular condition or the nervous condition.
Feature 13: The method of feature 11, wherein the subject is a postmenopausal woman who has experienced a decline in estrogen levels, which may contribute to an increased risk of developing neuromuscular or nervous conditions, and who may benefit from the neuroprotective and neuroregenerative effects of Clopidogrel, Ticagrelor, Prasugrel, and/or Cangrelor in reducing the number of denervated NMJs and promoting the reinnervation of NMJs, thereby improving neuromuscular function and quality of life.
Feature 14: The method of feature 11, wherein the subject is a pregnant woman who is at an increased risk of developing neuromuscular or nervous conditions due to the physiological changes and hormonal fluctuations associated with pregnancy, and who may benefit from the safe and effective use of Clopidogrel, Ticagrelor, Prasugrel, and/or Cangrelor in reducing the number of denervated NMJs and promoting the healthy development and function of NMJs, thereby ensuring the well-being of both the mother and the fetus.
Feature 15: The method of feature 11, wherein the pharmaceutically acceptable forms of one or more of Clopidogrel, Ticagrelor, Prasugrel, and/or Cangrelor comprise one or more of a salt of the one or more, a prodrug of the one or more, a hydrate of the one or more, a solvate of the one or more, or a combination thereof.
Feature 16: The method of feature 11, wherein the neuromuscular condition is selected from the group consisting of amyotrophic lateral sclerosis (ALS), spinal muscular atrophy (SMA), Duchenne muscular dystrophy, Becker muscular dystrophy, myasthenia gravis, Charcot-Marie-Tooth disease, spinal cord injury, a condition due to the normal aging process, nerve damages, nerve injuries, damages due to strenuous physical activities, fatigue, or other conditions that affect synapses as well as skeletal muscles.
Feature 17: The method of feature 11, wherein the nervous condition is selected from the group consisting of Alzheimer's disease, Parkinson's disease, Huntington's disease, multiple sclerosis, amyotrophic lateral sclerosis (ALS), spinal muscular atrophy (SMA), Charcot-Marie-Tooth disease, Friedreich's ataxia, Lewy body dementia, vascular dementia, frontotemporal dementia, progressive supranuclear palsy, corticobasal degeneration, chronic traumatic encephalopathy, Creutzfeldt-Jakob disease, and other neurodegenerative disorders.
Feature 18: The method of feature 11, wherein the effective amount of the one or more of Clopidogrel, Ticagrelor, Prasugrel, and/or Cangrelor or its pharmaceutically acceptable forms is a therapeutically effective amount that is determined based on a comprehensive assessment of the subject's medical history, the severity and progression of the neuromuscular or nervous condition, the presence of comorbidities, the concomitant use of other medications, and the individual pharmacokinetic and pharmacodynamic profile of the subject, and is adjusted as necessary to achieve the optimal balance between therapeutic efficacy and safety, while minimizing the risk of adverse effects and drug interactions, and is administered in a personalized dosing regimen that takes into account the subject's age, weight, gender, and other relevant factors, and is monitored and modified as needed to ensure the best possible outcomes for the subject.
Feature 19: The method of feature 11, wherein the administering is performed via a route selected from the group consisting of oral, parenteral, subcutaneous, intravenous, intramuscular, intraperitoneal, transdermal, intranasal, and rectal administration.
Feature 20: The method of feature 11, wherein the one or more of Clopidogrel, Ticagrelor, Prasugrel, and/or Cangrelor or a pharmaceutically acceptable forms is administered in a dosage form optimized for the selected route of administration, the dosage form being selected from the group consisting of a tablet, a capsule, a solution, a suspension, an emulsion, a powder, a granule, a suppository, an injection, an infusion, and an implant.

Other implementations are also described and recited herein. These and other features and advantages will be apparent from a reading of the following detailed description and a review of the associated drawings. It is to be understood that both the foregoing general description and the following detailed description are explanatory only and are not restrictive of aspects as claimed. The technology solves a long-felt but unmet need in the face of failure by others as is evidenced by Lou Gehrig's disease and the long history publicly associated with such a tragic condition as such the technology herein brings hope to many people.

### BRIEF DESCRIPTION OF THE DRAWINGS

Solely for the purpose of illustration, certain embodiments of the present invention are explained using examples in the drawings described below. It should be understood, however, that the invention is not limited to the precise arrangements, dimensions, and configurations shown. In the drawings:

FIG. 1A shows a schematic of the neuromuscular junction (NMJ) that includes perisynaptic Schwann cells (PSCs) ensheathing the motor axon. FIG. 1B shows images showing NMJs in a healthy young adult and from a symptomatic SOD1^{G93A} mouse, a model of ALS. Note that the motor axon is present in the healthy (WT) (at left) but absent from the SOD1^{G93A} NMJ.

FIG. 2A shows PSCs (yellow arrows, or now lighter grey arrows) and all other SCs (green arrows, or now darker grey) in skeletal muscle of an S100B-GFP;NG2-dsRed transgenic mouse. PSCs uniquely express a combination of NG2 (red, or now lighter grey) and S100B (green, or now darker grey) and are localized to NMJs (blue, or now grey as rendered). All other SCs express S100B, with most representing myelinating SCs that cover motor axons (not shown). FIG. 2B shows how P2Y12 protein (magenta, or now grey) is expressed in PSCs (yellow arrows, or now brighter grey) at the NMJ but not in myelinating Schwann cells (green arrows, or now darker grey as rendered). Scale bar=10 μm.

FIG. 3A shows representative images of AChRs (blue, or now grey on black) and synaptotagmin-labeled axons (red, or now lighter grey) in the EDL of P120 SOD1^{G93A} mice treated with Vehicle (top) or the P2Y12 antagonist Clopidogrel (bottom). FIG. 3B shows analysis of NMJ innervation revealed fewer fully denervated NMJs in Clopidogrel-treated SOD1^{G93A} mice. FIG. 3C shows clopidogrel-treated SOD1^{G93A} mice also had higher TA muscle mass. FIG. 3D shows a graphic example demonstrating a proposed role of Clopidogrel in inhibiting P2Y12 expressed by PSCs to promote the preservation of innervation in ALS mice. P2Y12 are the blue rectangles (or grey rectangles) on PSCs at the NMJ, which are inhibited by Clopidogrel (red X's or grey X's shown at right). For these FIGs., *p<0.05 versus vehicle; n=7-8. FIG. 3E shows Kir4.1 (green, or now darker grey) localizes to Sox10-Cre+ PSCs (red) at NMJs (labeled with α-BTX) in young adult Sox10-Cre;Stop-tdTom muscle. FIG. 3F shows tdTom (red, or now lighter grey) expressed under the Kir4.1 promoter localizes to NMJs (arrowhead) in Kir4.1-CreER;Stop-tdTom muscle. Arrow denotes extra junctional labeling in the phrenic nerve.

FIG. 4A and FIG. 4B provide data showing how Clopidogrel-treated SOD1^{G93A} mice had moderately improved gait parameters for stride frequency and FIG. 4C with FIG. 4D provide data for swing duration. FIG. 4E and FIG. 4F provide data showing that Clopidogrel treated SOD1^{G93A} mice also had improved braking which is important for generating force for propulsion. FIG. 4G and FIG. 4H show data for forelimb % stride in braking and hindlimb % stride in braking. *p<0.05, **p<0.01 versus vehicle; n=3-5. In FIGS. 4A-4H, DigiGait™ was used for gate analysis of P120 SOD1^{G93A} mice treated with Vehicle or the P2Y12 antagonist Clopidogrel. FIG. 4I shows fBTX-labeled AChRs and NG2⁺/S100B⁺ PSCs in P120 NG2-dsRed;S100B-GFP (WT) and NG2-dsRed;S100B-GFP;SOD1^{G93A} EDL. Analysis of SOD1^{G93A} muscle shows more PSCs/NMJ, reduced NMJ coverage (arrowhead), increased bridges between NMJs (arrows), and sprouts (asterisks). * p<0.05 versus WT. Scale bar=10 μm.

FIG. 5A illustrates using the neurological scoring system (NeuroScore) which is used to determine disease stage in SOD1^{G93A} mice. NeuroScore involved observing the ability of mice to control the position and mobility of both hindlimbs independently of each other. In this 0-4 point scoring system, 0 represents normal control and 4 represents complete loss of the ability to command hindlimb movement (FIG. 5A). SOD1^{G93A} mice were examined at 15 and 30 days after treatment with Clopidogrel and Vehicle starting at 90 days of age. NeuroScore revealed that the disease progressed slower in Clopidogrel treated compared to vehicle SOD1^{G93A} mice (see FIG. 5B).

FIG. 6A and FIG. 6B show representative NMJs from P190 WT and PFN1^{G118V} mice with arrow heads showing lack of PSC coverage, white arrow showing a PSC sprout, and orange (light grey) arrow showing partial denervation. FIG. 6C shows the innervation status of 30 NMJs from female P135, P170, and P190 WT and PFN1^{G118V} mice was determined. An NMJ was classified as innervated if >95% of the nAChRs was covered with SV2/2H3 staining, partial if 5-95% was covered, and denervated if <5% was covered. Two-way ANOVA ***p<0.001, ****p<0.0001 comparing fully innervated NMJs and § § § p<0.001, § § § § p<0.0001 comparing partially denervated NMJs. FIG. 6D shows the number % of NMJs with PSC sprouts and FIG. 6E shows incomplete PSC coverage was analyzed over 30 NMJs per mouse. FIG. 6F and FIG. 6G show how neurofilament light chain levels were measured monthly in male (FIG. 6F) and female (FIG. 6G) WT and PFN1^{G118V} mice. Statistical significance was determined using multiple unpaired t-tests with Welch's correction. Significance levels: *p>0.05, ***p<0.001, and ****p<0.0001. Scale bar=10 μm.

FIG. 7A shows a plot demonstrating P2ry12 transcripts are enriched in PSCs. FIG. 7B provides a plot of the analysis of P2Y12 fluorescence intensity in GFP-labeled PSCs in EDL of WT and SOD1^{G93A} S100B-GFP mice following P2Y12 IHC. Each data point represents 1 NMJ. FIG. 7C shows representative images of P2Y12 IHC WT and SOD1^{G93A} S100B-GFP EDL. Scale bar=10 μm. FIG. 7D shows a timeline of NMJ degeneration and symptom onset in SOD1^{G93A} and PFN^{G118V} ALS mouse models.

FIG. 8A shows representative images of AChR (Blue, or now rendered to grey), synaptotagmin-labeled axons (red, or now grey), and PSCs (green, or now grey) in the EDL of P115 SOD1^{G93A};S100B-GFP mice treated with Vehicle or the P2Y12 antagonist Clopidogrel. Fewer denervated NMJs and more PSC sprouts were observed in Clopidogrel treated SOD1^{G93A};S100B-GFP mice. FIG. 8B shows analysis of SOD1G93A muscle shows more NMJs where PSCs are extending sprouts and FIG. 8C shows reduced PSC coverage of the NMJ (see arrowhead in FIG. 4I). *p<0.05, **p<0.01, ***p<0.001, ****p<0.0001 versus WT. Scale bar=10 μm.

FIG. 9A, FIG. 9B, FIG. 9C, and FIG. 9D show electron micrographs of NMJs in WT and SOD1^{G93A} EDL with summary table shown in FIG. 9E. Scale bars=1 μm.

FIG. 10A, FIG. 10B, and FIG. 10C show IHC visualization and analysis showing P2Y12 (red in the merged image, or different shade of grey) increased at NMJs of the TA muscle of SOD1^{G93A} compared to WT mice at P110. (D) qPCR evaluation of P2Y12 mRNA levels in pre symptomatic (P70) and symptomatic (P110) SOD1^{G93A} TA muscles. *p<0.05 versus age-matched control; n=7 for IHC and 3-4 for qPCR. Scale bar=5 μm. FIG. 10D shows transcriptional analysis shows P2Y12 starting to increase in the presymptomatic and becoming significantly elevated in the early symptomatic phase of ALS pathogenesis in SOD1^{G93A} mice. FIG. 10E shows Table 1 illustrating Previous Aim 1B Experimental Design and plan that we will cross the Ki4.1-CreER;Ai14 line with a conditional P2Y12 line (P2Y12^{fl/fl}) 50 to delete P2Y12 from PSCs. A second cross will be made with SOD1^{G93A} mice. FIG. 10F shows Table 2 illustrating (previous plan) we will use 10 mice per genotype, age and sex for these experiments. Statistical comparisons will be made between tamoxifen-treated Kir4.1-CreER mice with and without P2Y12^{fl/fl} and vehicle treated Kir4.1-CreER; P2Y12^{fl/fl} mice1.

FIGS. 11A-11F provide a summary of Clopidogrel treatment on mouse model of ALS. FIG. 11A (control) and FIG. 11B provide images showing neuromuscular junctions (NMJs) treated with vehicle (control) and Clopidogrel. Blue depicts axons and Magenta muscle region in contact with motor axons. FIG. 11C and FIG. 11E are plots showing muscle regions are more intact in mice treated with Clopidogrel (“Clopid”). FIG. 11D and FIG. 11F are plots showing Clopidogrel reduces the number of denervated NMJs, and hence slows a hallmark of ALS pathogenesis. FIG. 11G provides representative FACS scatter plots showing the number of single-labeled S100B-GFP⁺ SCs (green) and double labeled S100B-GFP⁺; NG2-dsRed⁺ PSCs FACS-isolated from P90 WT and SOD1^{G93A} S100B-GFP;NG2-dsRed mice. FIG. 11H shows images of FACS-isolated PSCs in cell culture show that PSCs collected from SOD1^{G93A} S100B-GFP;NG2-dsRed have robust expression of both GFP and dsRed.

All trademarks, images, likenesses, words, and depictions in the drawings and the disclosure are plainly in fair use and are provided solely for the purposes of illustration of the invention in view of need to save lives and/or treat diseases as further discussed in detail below, even if unintentionally placed herein.

### DETAILED DESCRIPTION OF THE INVENTION

The subject innovation is now described in some instances, when necessary, with reference to the drawings. In the following description, for purposes of explanation, numerous specific details are set forth in order to provide a thorough understanding of the present invention. It may be evident, however, that the present invention may be practiced without these specific details. In other examples, well-known structures, methods, and devices are shown in diagram form or with illustrations in order to facilitate describing the present invention. It is to be appreciated that certain aspects, modes, embodiments, variations and features of the invention are described below in various levels of detail in order to provide a substantial understanding of the present invention.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. This disclosure is purposefully in commonly understood words, known to a person of skill in the art, but Merriam-Webster's Online Dictionary is used, when appropriate, for terms not specifically demonstrated herein or not known in the art.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a value or parameter are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). As used herein, reference to “approximately” or “about” a value or parameter includes (and describes) embodiments that are directed to that value or parameter. For example, description referring to “about X” includes description of “X”.

As used herein, the term “or” means “and/or.” The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” means that other elements can also be present in addition to the defined elements presented. The use of “comprising” indicates inclusion rather than limitation. The term “including” can be interchanged with “comprising”.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention. For example, a pharmaceutical formulation can consist essentially of Clopidogrel and another therapeutic agent, meaning that a variety of excipients or other additives can be present in the formulation, but no other active pharmaceutical ingredient (API) is present in the formulation, except in formulations wherein an intended synergistic effect is demonstrated by the claims or examples herein. In another example, a pharmaceutical formulation can consist essentially of Clopidogrel, meaning that the formulation is provided in the form of a nasal spray, an inhaled formulation, an orally administered formulation, or an injection formulation, each of which is tailored for a fast-acting agent, therapeutic agent, or treatment but not tailored for long-term administration (e.g., as a simultaneous treatment). In another example, in the case of a preventative therapy to purposefully prevent a condition, the opposite, long-term combination therapy, can be referred to with “consisting essentially of”. This example could be applied to a patient who is best treated by an evolving combination therapy. In another example, the term “consisting essentially of” can also be exemplified by plain language provided in the claims.

The term “statistically significant” or “significantly” refers to statistical significance and generally means a two-standard deviation (2SD) or greater difference.

As used herein, the term “subject” refers to a mammal, including but not limited to a dog, cat, horse, cow, pig, sheep, goat, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects particularly include human subjects in urgent treatment as described herein. The human subject may be a pediatric, adult, or a geriatric subject. The human subject may be of any sex.

As used herein, the terms “effective amount”, “therapeutically effective amount”, and “pharmaceutically effective amount” include an amount sufficient to prevent or ameliorate a manifestation of or a suspected manifestation of a medical condition. The manifestation can be a sign or symptom or otherwise. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the medical condition and on the characteristics of the individual, such as general health, age, sex, body weight and tolerance to drugs. It will also depend on the degree, severity, and type of medical condition. The skilled artisan will be able to determine appropriate dosages depending on these and other factors. Examples of other factors can be route of administration and length of administration(s). The compositions can also be administered in combination with one or more additional therapeutic compounds.

As used herein, a therapeutic that “prevents” a disorder or condition refers to a compound (or combination) that, in a statistical sample, reduces the occurrence of the disorder or condition in the treated sample relative to an untreated control sample, or delays the onset or reduces the severity of one or more symptoms of the disorder or condition relative to the untreated control sample. For example, a compound (or combination) that prevents epilepsy may reduce the frequency of seizures and/or reduce the severity of seizures.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The phrases “conjoint administration” and “administered conjointly” refer to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered at the same time, within one minute, 2 minutes, 4 minutes, 6 minutes, 10 minutes, 30 minutes, or an hour or 90 minutes of one another. In some embodiments, the different therapeutic compounds can be administered within 1 year of one another. Thus, an individual who receives such treatment can benefit from a combined effect of different therapeutic compounds.

As used herein, the terms “treat,” “treatment,” “treating,” or “amelioration” when used in reference to a disease, disorder, or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), sign(s), diminishment of extent of the deficit, stabilized (i.e., not worsening) state of a symptom or condition, delay or slowing of onset of symptoms or indications, and an increased lifespan as compared to that expected in the absence of treatment.

As used herein, the term “long-term” administration means that the therapeutic agent or drug is administered for a period of at least 12 weeks. This includes that the therapeutic agent, combination, or drug is administered such that it is effective over, or for, a period of at least 12 weeks and does not necessarily imply that the administration itself takes place for 12 weeks, e.g., if sustained release compositions or long-acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

The administration of the compositions contemplated herein may be carried out in any convenient manner, including by aerosol inhalation, injection, ingestion, transfusion, implantation, application (e.g., topical, otic, or ocular), or transplantation. Administration can be accomplished by an implant. In some embodiments, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into an artery, vein, lymph node, or organ (e.g., heart, muscle, organ).

The terms: “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The term “prodrug” is intended to encompass compounds which, under physiologic conditions, are converted into the therapeutically active agents of the present invention (e.g., any compound selected from this disclosure). A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present invention. In certain embodiments, some or all of the compounds selected from this disclosure in a formulation can be replaced with the corresponding suitable prodrug, e.g., wherein a hydroxyl in the parent compound is presented as an ester or a carbonate or carboxylic acid present in the parent compound is presented as an ester. In some embodiments, a “prodrug” is made by using an absorbing particle that subsequently releases an active form after administration.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, an agent or a therapeutic agent provided to a subject and suspected to be or involved in a treatment can be a small molecule less than 1000 MW or a large molecule not less than 1000 MW including biologics, oligonucleotides, peptides, oligosaccharides, and larger molecules. Any of the therapeutic agents disclosed herein can be used as or in combination with small molecules and/or large molecules as discussed herein.

As used herein, a subject may or may not be aware of suffering from a condition. A health care provider may suspect the condition or may have confirmed the condition.

A subject can be one who has been previously diagnosed with or identified as suffering from or having a condition in need of treatment (e.g., a nerve injury or related disorder) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition, or one or more complications related to a condition or a subject who does not exhibit risk factors. A “subject in need” of treatment for a particular condition can be a subject having that condition, diagnosed as having that condition, suspected as having, or at risk of developing that condition. In another example, the subject has been brought into a treatment situation entirely without the subject's knowledge and/or intent. For example, a subject can obviously be in need of treatment but not be responsive to a treatment, and as described herein the present methods and formulations may save the subject's life.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art. See, e.g., Isselbacher, et al., (1996)(Isselbacher, Braunwald et al. 1996).

As discussed above, unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only and is not intended to limit the scope of the present invention, which is defined solely by the claims. Definitions of common terms in immunology and molecular biology can be found in The Merck Manual of Diagnosis and Therapy; (Porter 2011) The Encyclopedia of Molecular Cell Biology and Molecular Medicine; (Robert S. Porter 1999-2012) Molecular Biology and Biotechnology: a Comprehensive Desk Reference; (Robert A. Meyers 1995) Immunology; (Luttmann 2006) Janeway's Immunobiology; (Kenneth Murphy 2014) Lewin's Genes XI; (Krebs, Lewin et al. 2014) Molecular Cloning: A Laboratory Manual.; (Green 2012) Basic Methods in Molecular Biology; (Davis 2012) Laboratory Methods in Enzymology; (Jon Lorsch 2013) Current Protocols in Molecular Biology (CPMB)(Frederick M. Ausubel 2014); Current Protocols in Protein Science (CPPS); (John E. Coligan 2005) and Current Protocols in Immunology (CPI)(John E. Coligan 2003). If there is an apparent discrepancy between the usage of a term in the art and its definition provided herein, the definition provided within the specification shall prevail. In general, chemical terminology is found in the International Union of Pure and Applied Chemistry GoldBook (IUPAC).

The term “protecting group” refers to a group of atoms that, when attached to a reactive functional group in a molecule, mask, reduce or prevent the reactivity of the functional group. Typically, a protecting group may be selectively removed as desired during the course of a synthesis. Examples of protecting groups can be found in Greene and Wuts, Protective Groups in Organic Chemistry, 3^{rd} Ed. (Wuts 1999) and in Harrison, et al., Compendium of Synthetic Organic Methods, Vols. 1-8 (Ian T. Harrison 1971-1996). Examples of representative nitrogen protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like. Examples of representative hydroxylprotecting groups include, but are not limited to, those where the hydroxyl group is either acylated (esterified) or alkylated such as benzyl and trityl ethers, as well as alkyl ethers, tetrahydropyranyl ethers, trialkylsilyl ethers (e.g., TMS or TIPS groups), glycol ethers, such as ethylene glycol and propylene glycol derivatives and allyl ethers.

In some embodiments, the decrease in the one or more signs or symptoms is evaluated according to the DSM-5 (Association 2013). In some embodiments, signs are observed or measured by a health care provider. Symptoms can be reported by the subject. In some embodiments, the decrease of signs or symptoms occurs in less than about 120 minutes, 90 minutes, less than about 60 minutes, less than about 30 minutes, less than about 15 minutes, less than about 10 minutes, or less than about 5 minutes, or less than about 3 minutes, or less than about 1 minute. In some embodiments, the decrease of signs or symptoms occurs in less than 1 day, less than 1 week, less than 1 month, or in less than 1 year.

### Pharmaceutical Compositions

The compositions and methods of the present invention may be utilized to prevent a need for other treatment, to provide benefit when other treatment(s) fail, or to treat an individual in need thereof. In some embodiments, the individual is suspected of needing treatment. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. A compound can represent a combination therapy herein. Pharmaceutically acceptable carriers are well known in the art and include, for example, aqueous solutions such as water or physiologically buffered saline or other solvents or vehicles such as glycols, glycerol, oils such as olive oil, or injectable organic esters. In some embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-micro emulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible compositions employed in pharmaceutical formulations.

A pharmaceutical composition (preparation) can be administered to a subject by any of a number of routes of administration including, for example, orally, for example, drenches as in aqueous or non-aqueous solutions or suspensions, tablets, capsules including sprinkle capsules and gelatin capsules, boluses, powders, granules, pastes for application to the tongue; absorption through the oral mucosa (e.g., sublingually); subcutaneously; transdermal administration (for example as a patch applied to the skin); and topically (for example, as a cream, ointment or spray applied to the skin). The compound may also be formulated for inhalation. Inhalation can include inhalation of a liquid (droplets or aerosol). Inhalation can include a micronized powder adhered to carrier particles or can be without carrier particles. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, all of which are incorporated herein in entirety by reference, and as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound (or combination therapy) of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules, including sprinkle capsules and gelatin capsules), tablets, pills, dragees, powders, granules and the like, the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropyl methyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropyl methyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymers and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, micro-emulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (e.g., in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intraocular (such as intravitreal), intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The Clopidogrel, Ticagrelor, Prasugrel, and/or Cangrelor can be attached to a targeting moiety. Examples of targeting moieties are antibody drug conjugates, nanoparticles, metals, polymers, nucleotides, peptides, and aptamers.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In other embodiments, the active compound will be administered once daily.

The subject or patient receiving this treatment is any animal in need, including primates, in particular humans; and other mammals such as equines bovine, porcine, sheep, feline, and canine; birds such as poultry; and pets in general.

In certain embodiments, compounds of the invention may be used alone or conjointly administered with another type of therapeutic agent. The another type of therapeutic agent can include therapies such as diet, exercise, application of radiation or electromagnetic waves, or an electrotherapy.

The present disclosure includes the use of pharmaceutically acceptable salts of compounds of the invention in the compositions and methods of the present invention. In certain embodiments, contemplated salts of the invention include, but are not limited to; bisulfate; chloride; alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, I-ascorbic acid, I-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, I-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, I-pyroglutamic acid, salicylic acid, sebacic acid, stearic acid, succinic acid, sulfuric acid, I-tartaric acid, thiocyanic acid, p-toluenesulfonic acid, trifluoroacetic acid, and undecylenic acid salts. In some embodiments, an N—H in Clopidogrel can become charged and a corresponding salt formed at or near the charge.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent. The invention contemplates polymorphs, cocrystals, and amorphous forms of all substances discussed herein. As discussed above, solvates and/or hydrates can be formed by, for example, a slow evaporation whereby water and/or solvent remain hydrogen bonded with OH groups in the molecule. The formation of a solvate/hydrate can be quickly confirmed after the evaporation by using attenuated total reflectance Fourier transform infra-red spectroscopy wherein the solid solvate/hydrate is directly placed on the instrument and the subsequent IR spectrum is compared to the IR spectrum of the solid non-solvate, non-hydrate.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In the embodiments discussed and in any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

A Novel Use of Clopidogrel/Plavix® (or Combinations Thereof) to Treat Amyotrophic Lateral Sclerosis, Damages in Caused by the Normal Aging Process, Nerve Injuries, Muscular Dystrophy, Strenuous Physical Activities and Other Conditions that Affect Synapses as Well as Skeletal Muscles.

Introduction: Clopidogrel is an FDA-approved (US Food and Drug Administration approved) drug that works by blocking the activity of a specific purinergic receptor called P2Y12. Our lab discovered P2Y12 expressed in a special type of Schwann cells, called perisynaptic Schwann cells. These cells are critical for the maintenance and repair of the synapse critical for the viability and function of skeletal muscles. We have gathered data showing that muscles maintain their synapses in a mouse model of Amyotrophic Lateral Sclerosis (ALS) treated with Clopidogrel but not vehicle. This finding indicate that Clopidogrel could be used to treat ALS and other conditions that cause instability and degeneration of muscle and other synapses. In place of the Clopidogrel can be used Ticagrelor, Prasugrel, and/or Cangrelor. Also, the Clopidogrel, Ticagrelor, Prasugrel, and/or Cangrelor can be used in any combination.

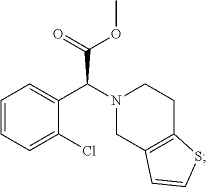 [SMILES: COC(=O)[C@H](c1ccccc1Cl)N1CCc2sccc2C1]

Example chemical structure of Clopidogrel shown above;

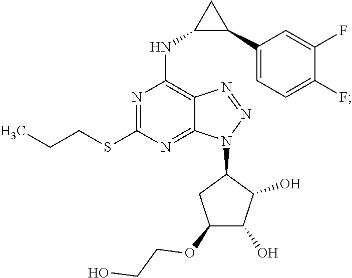 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

Example chemical structure of Ticagrelor shown above;

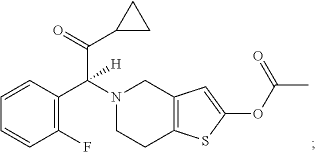 [SMILES: CC(=O)Oc1cc2c(s1)CCN([C@H](C(=O)C1CC1)c1ccccc1F)C2]

Example chemical structure of Prasugrel shown above;

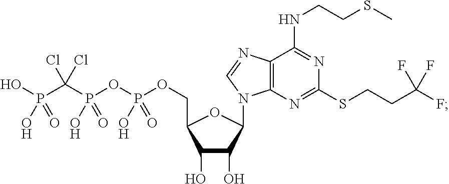

Example chemical structure of Cangrelor shown above.

The chemical structures shown above are non-limiting because (as discussed herein) all tautomers, isomers, salts, etc. are contemplated in the spirit of the Invention (namely, to save human lives). In some embodiments, this invention can provide treatments for conditions that adversely affect synapses that include ALS, Alzheimer's Disease, nerve injuries, muscular dystrophy, aging, strenuous physical activities. To date, there are no effective treatments for any of those conditions. Hence, this invention would address major health issues affecting millions of lives.

We propose to use Clopidogrel, Ticagrelor, Prasugrel, and/or Cangrelor to treat ALS and other conditions that cause synapses to degenerate. One or more of the small molecules (example structures shown above) would be administered patients with ALS, Alzheimer's Disease, muscular dystrophy, recovering from nerve injuries, strenuous physical activities, and aging.

Treatments for ALS, Alzheimer's Diseases, recovery of nerve injuries and strenuous physical activities are either only moderately effective or do not exist. Thus, Clopidogrel could be a better treatment alone or used in a combinatorial manner with existing treatments for conditions that cause synaptic instability and degeneration. The drug is already approved by the FDA and routinely used orally. Hence, the barriers to get this drug to patients suffering from conditions that cause synapses to degenerate are minimum.

### Research Strategy

A. SIGNIFICANCE: Amyotrophic Lateral Sclerosis (ALS) is an adult-onset neurodegenerative disease that targets motor neurons, causing paralysis and death within 5 years of diagnosis[[1,2]]. ALS affects ˜1:40,000 Americans and incurs an immense emotional and financial burden to patients and their families. Although the disease has been clinically recognized for over 140 years and multiple genetic factors have been identified[[3]], effective therapies for ALS have yet to be developed. Riluzole, Radicava (Edaravone), Relyvrio, and Qalsody, the only therapies approved for ALS, are minimally effective, only extending life by several months[[4-7]]. Thus, there is a pressing need to identify new and more efficacious therapies for ALS.

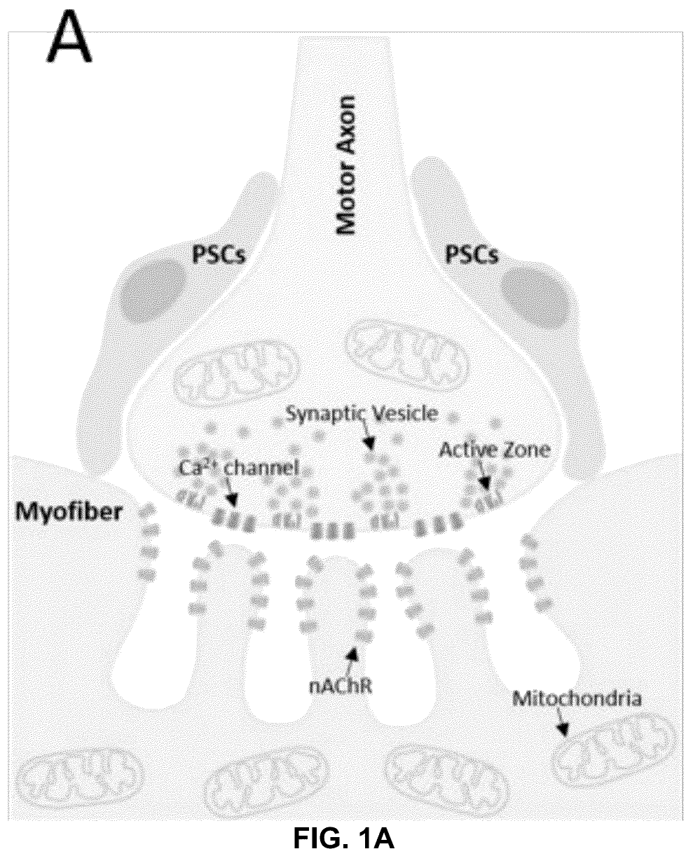

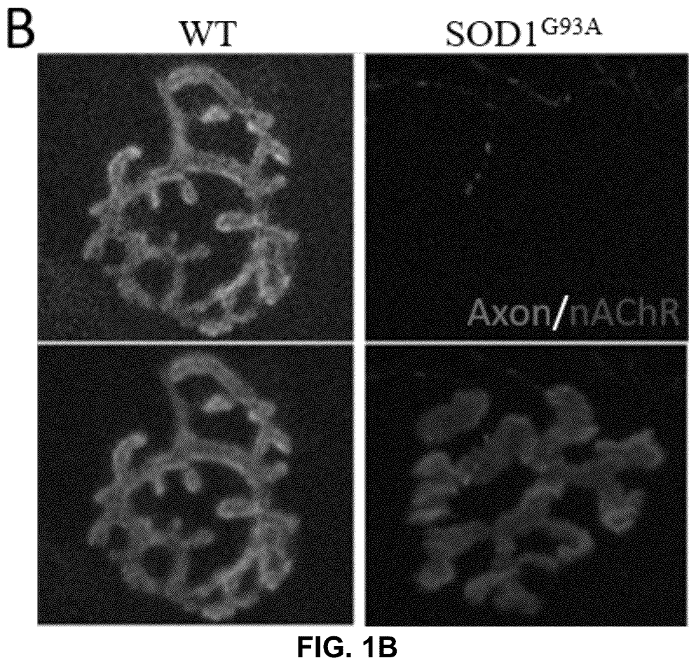

The minimal impact of these drugs is partially due to their administration after symptom onset, which occurs only after the majority of motor neurons have been lost. Thus, there is a pressing need to identify targets that will prevent motor neuron death during the earliest stages of ALS. One attractive target is the interface between motor neurons and skeletal muscles, known as the neuromuscular junction (NMJ) (FIG. 1A). This synapse is necessary for all voluntary movements[[8]] and its deterioration is an early pathological feature of ALS (FIG. 1B) thought to contribute to motor neuron death[[9]]. Studies in ALS patients and animal models have revealed that a large portion of denervated NMJs become reinnervated by surviving motor neurons[[10-12]], underscoring the regenerative potential of the lower motor system. Ongoing clinical trials of drugs that target the NMJ to treat related motor neuron diseases, such as spinal muscle atrophy[[13]], show promise, however no such therapies have been developed for ALS. Thus, as an accessible synapse located outside of the blood brain barrier, the NMJ is an attractive therapeutic target to prevent or slow motor neuron degeneration and muscle atrophy in ALS[[9]].

The NMJ is a tripartite cholinergic synapse (FIG. 1A) comprised of an α-motor neuron (the presynapse), the specialized region on an extrafusal muscle fiber (the postsynapse), and specialized glia called perisynaptic Schwann cells (PSCs)[[14]]. Extensive effort has been devoted to understanding the initial cellular and molecular changes that precipitate NMJ degeneration and repair. However, most studies, to date, have primarily focused on the pre- and postsynapse due to the ease with which skeletal muscles and motor axons can be identified and targeted. This has given us a clearer understanding of the molecular and cellular changes associated with ALS in the pre- and postsynaptic regions of the NMJ. In stark contrast, little is known about how PSCs contribute to NMJ degeneration in ALS. FIG. 1A shows a schematic of the neuromuscular junction (NMJ) that includes perisynaptic Schwann cells (PSCs) ensheathing the motor axon. FIG. 1B shows images showing NMJs in a healthy young adult and from a symptomatic SOD1^{G93A} mouse, a model of ALS. Note that the motor axon is present in the healthy (WT) (at left) but absent from the SOD1^{G93A} NMJ.

Rigor of the Research: Accumulating evidence suggests that perisynaptic Schwann cells (PSCs) play roles in the degeneration of NMJs in ALS. PSCs are specialized non-myelinating Schwann cells that are integral components of the NMJ, and thus bona-fide synaptic glial cells. PSCs maintain and repair NMJs in healthy animals[[14-16]]. They do so by surveying and modulating neurotransmission, and becoming repair cells that clear axonal debris and promote reinnervation of previously denervated NMJs[[16,17]]. But in ALS, PSCs have been found with a number of abnormalities that include altered sensitivity to synaptic transmission[[18]], migration away from the NMJ[[19]], aberrant insertion of processes into the synaptic cleft[[20]], and impaired phagocytosis of degenerating axonal debris[[21]]. These functional changes in PSCs are similar to those seen in glia of the central nervous system (CNS) in ALS. While CNS glia have been directly implicated in ALS progression[[22]], it is unknown if these changes in PSCs ultimately compromise the health of NMJs or motor neurons in ALS. There is even less known about molecular mechanisms in PSCs that could serve as therapeutic targets to promote NMJ stability and repair. This information could lead to treatments for ALS that target PSCs. We have generated data showing that PSCs transition to a reactive phenotype in the early stages of ALS progression that may contribute to NMJ degeneration.

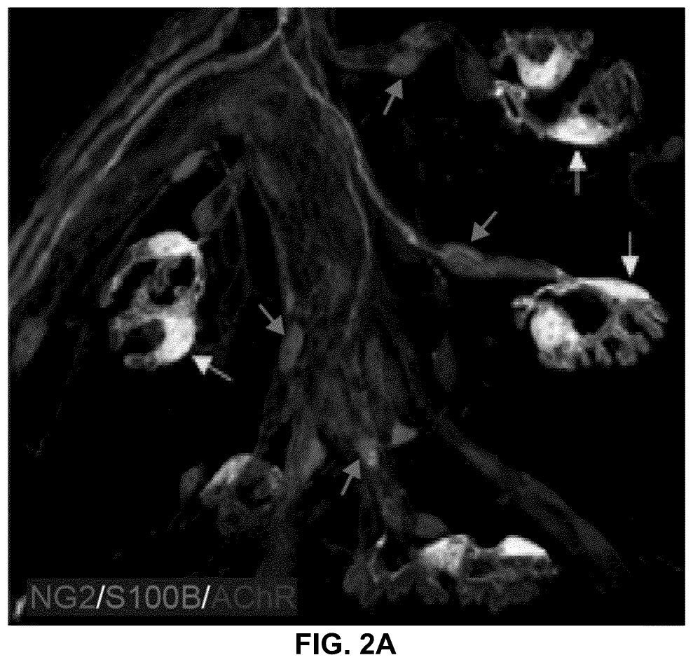

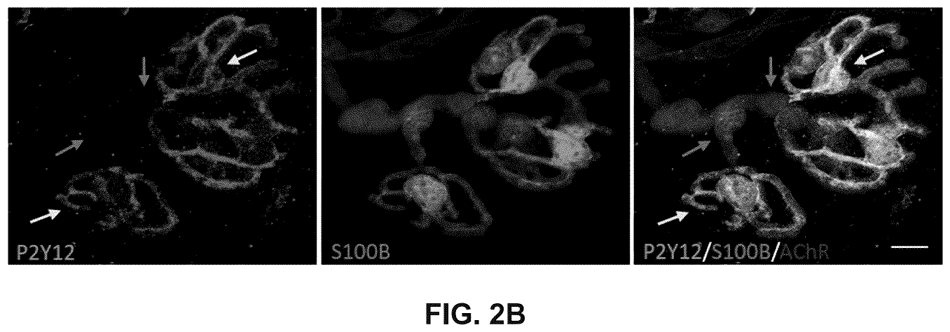

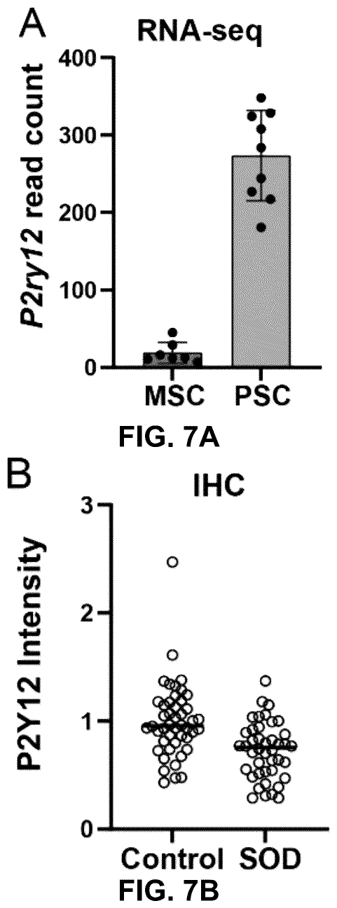

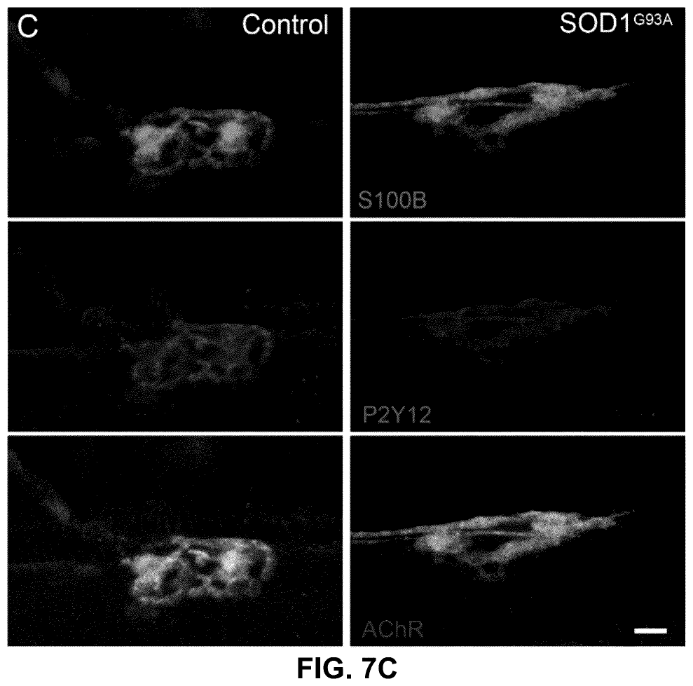

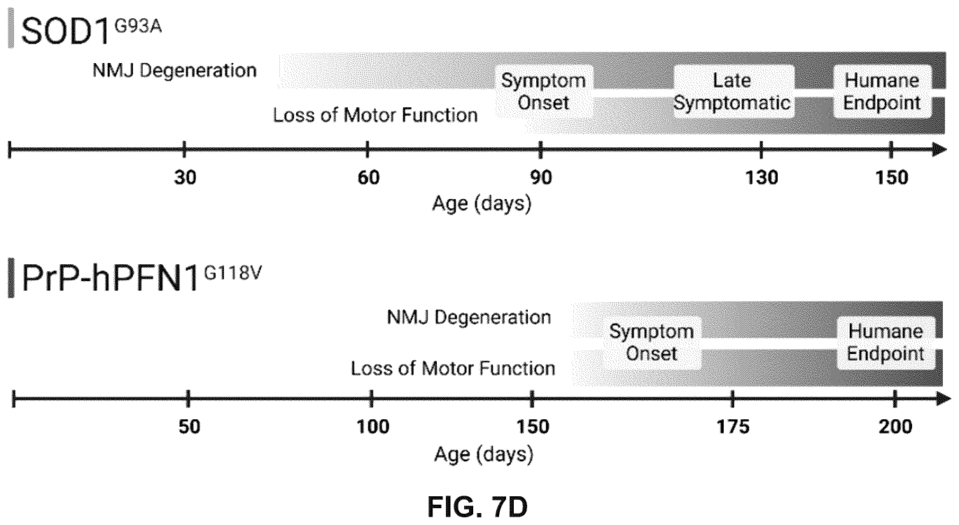

Until recently, there was even less known about molecular mechanisms in PSCs that could serve as therapeutic targets to promote NMJ stability and repair. This gap in our knowledge was primarily due to the lack of genetic tools to specifically identify and target molecular mechanisms in PSCs. To overcome this limitation, our lab recently developed a new mouse transgenic line and method to isolate PSCs from all other SCs to interrogate their gene expression profile[[23]](e.g., transgenic mice FIG. 2A). These new developments led us to discover the purinergic receptor P2Y12 is primarily expressed by PSCs among Schwann cells and other skeletal muscle resident cells (FIG. 2B, FIG. 7A). In addition, we were led to discover that the purinergic receptor P2Y12 is enriched in PSCs (FIG. 7A). This finding is significant because activity-guided transitions between maintenance and repair modes in PSCs are thought to be acutely controlled by purinergic receptors in ALS-afflicted NMJs[[18,24]] and by both purinergic and muscarinic receptors in healthy NMJs[[25,26]]. Further highlighting the potential importance of purinergic signaling in ALS-afflicted muscle, this signaling pathway has been shown to mediate PSC axonal pruning and NMJ remodeling[[27]]. Purinergic signaling has also been shown to play important functions in mediating the homeostatic actions of glial cells in the central nervous system[[28-31]]. Additionally, purinergic signaling has been demonstrated to impact cardiovascular health and inflammation in peripheral tissues as well as fibrosis in skeletal muscles[[32,33]]. Because of these published findings and our discovery that P2Y12 is enriched in PSCs, we have begun to examine purinergic signaling in PSCs with ALS.

In experiments, we have generated data indicating that P2Y12 is dysregulated in PSCs of SOD1^{G93A} mice, a mouse model of ALS. We found P2Y12 expression elevated in SOD1^{G93A} mice before they exhibit significant ALS-related pathology, such as the degeneration of NMJs. Importantly, we have obtained initial exciting data showing that inhibiting P2Y12 with Clopidogrel, an FDA approved drug commonly known as Plavix®, prevents NMJ degeneration, preserve muscle mass and improves motor function in SOD1^{G93A} mice. As discussed above, Clopidogrel is a blood thinner taken orally on a daily basis by patients at risk of heart attack and stroke. We have postulated that attenuating P2Y12 signaling in PSCs will protect NMJs from degeneration in ALS. In addition to PSCs, we will examine the phenotype of microglia in mouse models of ALS treated with Clopidogrel because they also express P2Y12 and are well-known to contribute to ALS pathogenesis. Overall, this is highly significant because it provides herein: 1) critical pre-clinical data to use Clopidogrel as a potential new treatment for ALS; 2) new insights into the role of PSCs in NMJ degeneration in ALS; 3) new leads regarding the role of P2Y12 signaling, including downstream effectors, in both PSCs and microglia; and 4) set the stage for additional future studies to modulate P2Y12 signaling in PSCs, microglia and other P2Y12 positive cells that include platelets to further advance basic and translational research to treat ALS.

We have obtained data showing that inhibiting P2Y12 slows NMJ degeneration in symptomatic SOD1^{G93A} mice. FIG. 2A shows PSCs (brighter grey arrows, previously were yellow when in color) and all other SCs (darker grey arrows, previously green) in skeletal muscle of an S100B-GFP;NG2-dsRed transgenic mouse. PSCs uniquely express a combination of NG2 (red, now brighter grey) and S100B (green, now darker grey) and are localized to NMJs (blue, now grey). All other SCs express S100B, with most representing myelinating SCs that cover motor axons (not shown). FIG. 2B shows how P2Y12 protein (magenta, now grey) is expressed in PSCs (yellow arrows, now brighter grey) at the NMJ but not in myelinating Schwann cells (green arrows, now darker grey). Scale bar=10 μm.

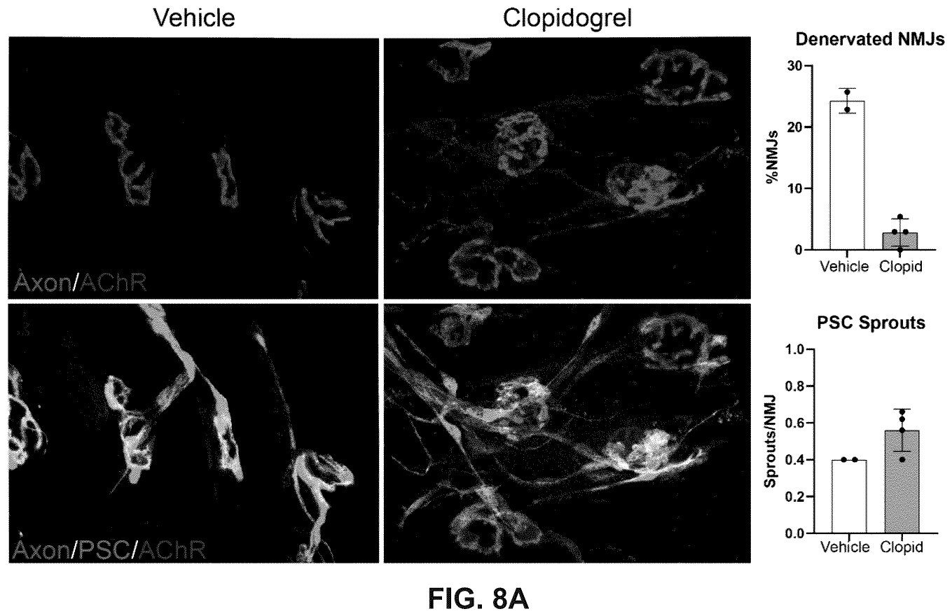

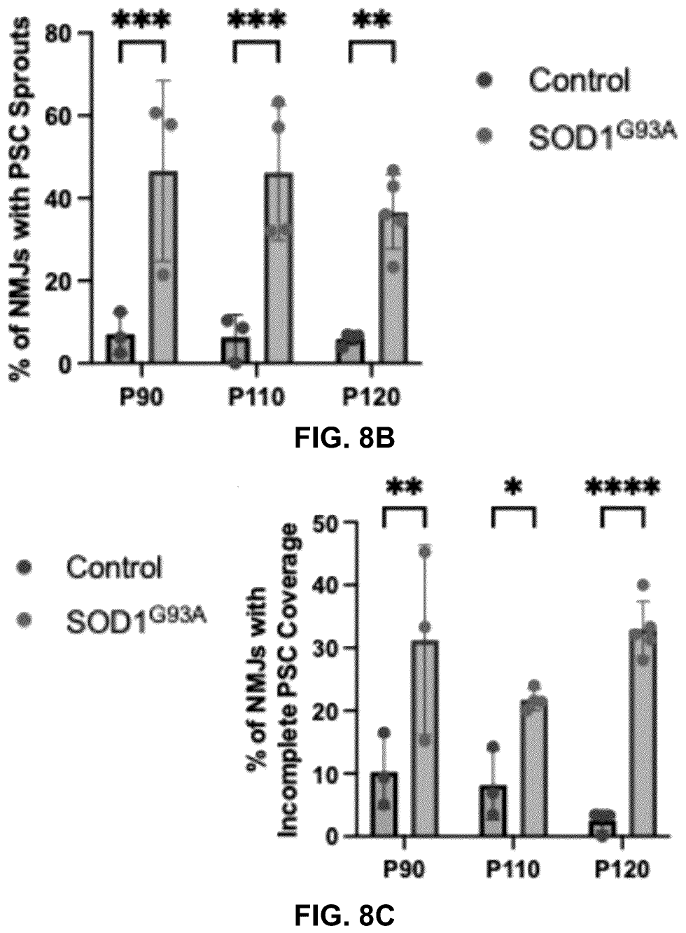

In this study, we define the role of P2Y12 in mediating the maintenance and repair activity of PSCs at NMJs afflicted with ALS. Additionally, we performed experiments to identify downstream effectors of P2Y12 that are dysregulated in PSCs afflicted with ALS. We carried out these experiments in both the SOD1^{G93A} and PFN1^{G118V} mouse models of ALS. These are well characterized models of ALS, shown to exhibit similar pathological symptoms as human patients with these ALS-causing mutations[[12,34]]. To further increase the impact of our discoveries, we validate promising findings in additional mouse models of the disease.
B. INNOVATION. This study is both conceptually and methodologically innovative for the following reasons: 1—Conceptual framework: This proposal is conceptually unique because it focuses on the contribution of synaptic Schwann cells to ALS-induced NMJ degeneration. It focuses on the contribution of synaptic glia to ALS-induced NMJ degeneration. It relies on genetic, cellular and molecular approaches along with the use of one or more pharmacological agents to ascertain the contribution of P2Y12 signaling in PSC-mediated NMJ degeneration in ALS. It will also use recently discovered markers of PSCs[[23]] to identify P2Y12 signaling dependent and independent pathways dysregulated in PSCs from mouse models of ALS and to generate additional molecular insights about downstream effectors of P2Y12 and other pathways that are dysregulated in PSCs affected by ALS. Additionally, it will examine the function of microglia using cellular and molecular approaches. These combined strategies will provide both basic and translational insights for treating ALS by targeting P2Y12 signaling. It will also set the stage for additional future studies to further fine-tune the activity of P2Y12 signaling and to target downstream effectors altered by Clopidogrel in PSCs and microglia in ALS. These combined strategies will provide both basic and translational insights for treating ALS by targeting a specific signaling module in PSCs to prevent NMJ degeneration.
2—Modulating purinergic signaling to promote NMJ maintenance and repair: This proposal is the first to examine the contribution of purinergic signaling mediated by P2Y12 in the destructive actions of PSCs and microglia in ALS. We are investigating purinergic signaling because it has been shown to guide the transition of glial cells, including PSCs at NMJs11, between maintenance and repair modes. Purinergic signaling has been shown to guide the transition of glial cells, including PSCs[[15]], between maintenance and repair modes. Relevant to ALS, purinergic signaling is important for PSC axonal pruning and NMJ remodeling[[27]]. Despite this information, the specific purinergic receptor(s) that mediate PSC responses in ALS have remained unclear. In a preliminary experiment, we used FACS and RNA-seq to identify several purinergic receptors in PSCs. We found P2Y12 to be highly enriched in these glial cells (FIG. 7A). We have collected preliminary data showing that P2Y12 mediates PSC function and slows NMJ degeneration in ALS (FIG. 8A). We will leverage this information along with an FDAapproved modulator of this receptor to test whether fine-tuning purinergic signaling in PSCs prevents NMJ and motor neuron degeneration in ALS. We will use the well-characterized SOD1^{G93A} and PFN1^{G118V} mouse models of ALS[[12,34,35]]. These mice were generated to express either human superoxide dismutase 1 (SOD1) or profilin 1 (PFN1) with mutations found to cause ALS, with symptoms beginning in the hindlimbs and progressing rostrally.
3—Molecular Markers and genetic tools to study PSCs independently of other SCs: We have recently a novel transgenic mouse line that enables us to separate PSCs from all other SCs (FIG. 2A) via FACS for gene expression analysis. We have used this tool to identify genes that are uniquely enriched in PSCs20 that include purinergic receptor P2Y12. We will use our new genetic tools to identify P2Y12 dependent and independent signaling pathways dysregulated in PSCs afflicted with ALS. PSCs have been implicated in ALS-pathogenesis[[18-21,36-39]] yet they have received little attention as potential targets to treat the disease. To date, there is a paucity of information about the intrinsic mechanisms behind PSC repair, making it difficult to determine if or how these cells become dysfunctional in ALS. This has been due to the inability to isolate PSCs from other SCs using a selective marker.

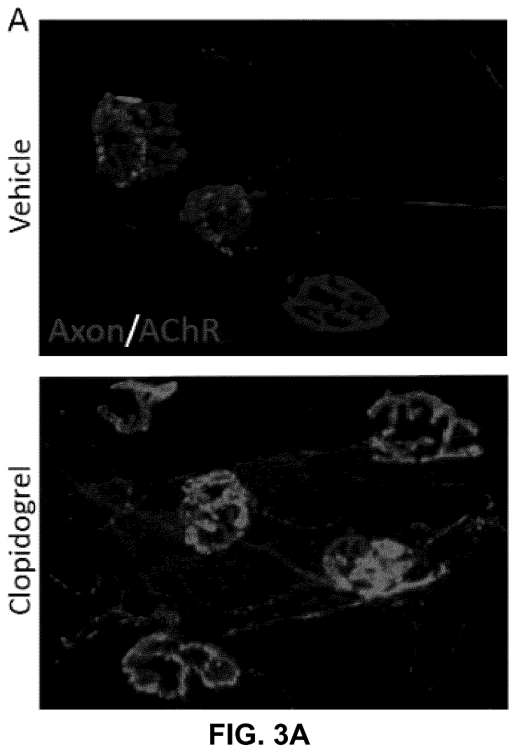

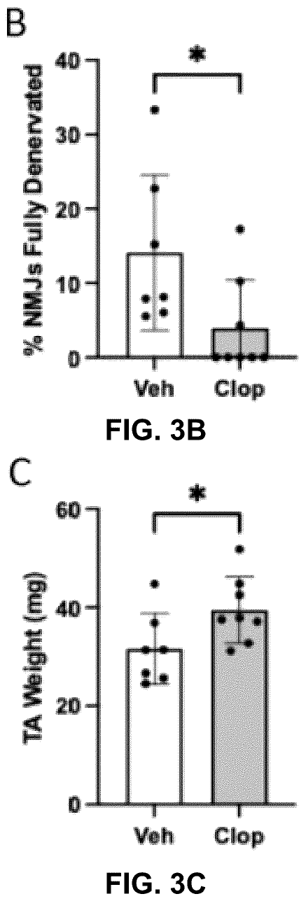

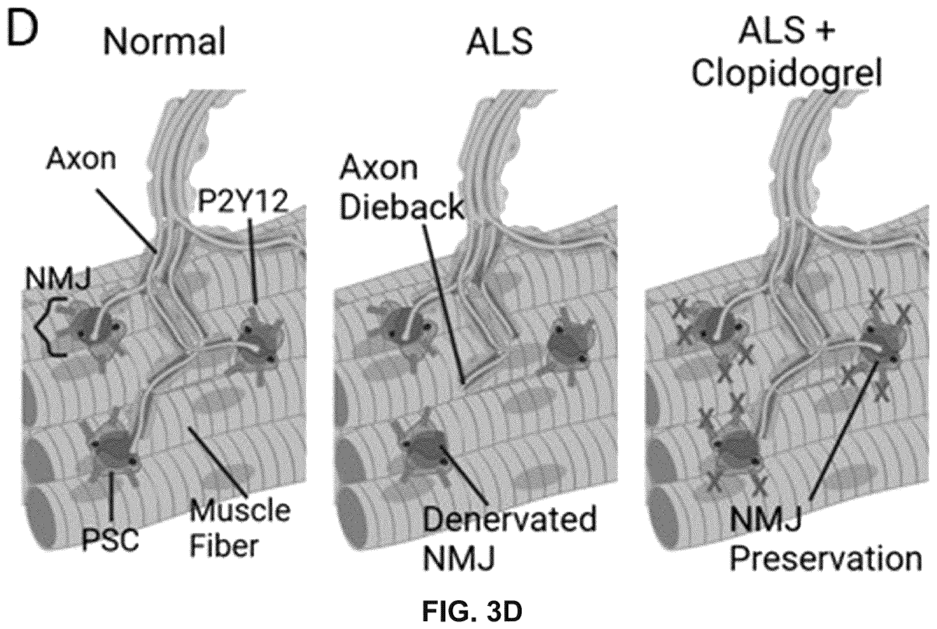

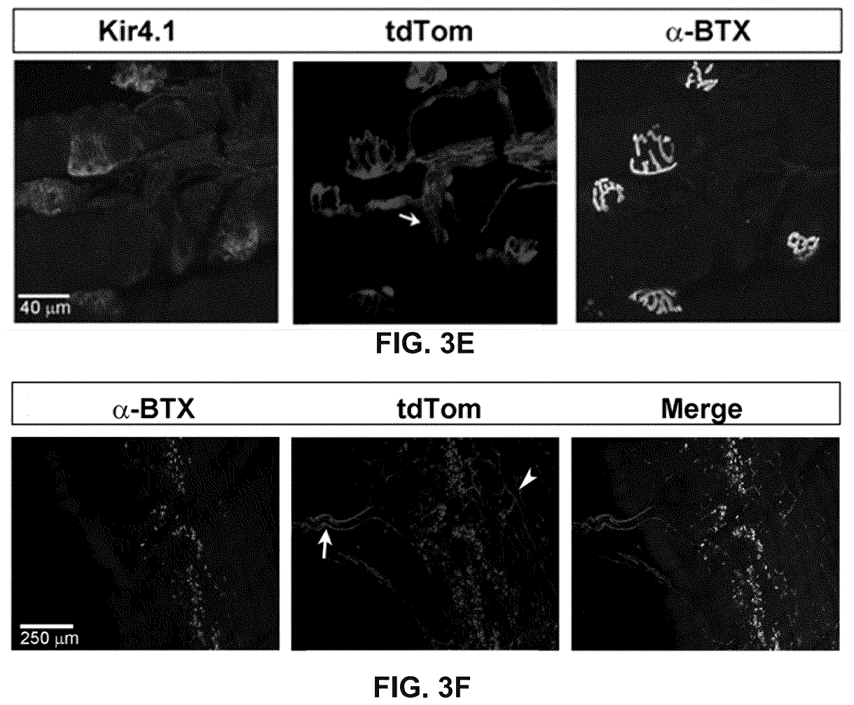

We have recently identified a unique marker of PSCs and developed a transgenic mouse line (FIG. 2A) that enables us to separate PSCs from all other SCs via FACS for gene expression analysis[[23]]. We have used this tool to identify genes that are uniquely enriched in PSCs[[23]]. This has led to the discovery of a set of purinergic receptors with well-established roles in neurodegeneration that are enriched in PSCs. Additionally, new genetic tools to genetically excise or introduce genes primarily in PSCs among all other cells residing in skeletal muscles have been generated[[40]](FIG. 3E, FIG. 3F). FIG. 3E shows Kir4.1 (green, or now darker grey) localizes to Sox10-Cre+ PSCs (red, or now lighter grey) at NMJs (labeled with α-BTX) in young adult Sox10-Cre;Stop-tdTom muscle. FIG. 3F shows tdTom (red, or now lighter grey) expressed under the Kir4.1 promoter localizes to NMJs (arrowhead) in Kir4.1-CreER;Stop-tdTom muscle. Arrow denotes extra junctional labeling in the phrenic nerve.
C. APPROACH: This work had, for example, three goals. First, it tests the hypothesis that Clopidogrel slows the initiation and progression of ALS in two different mouse models of the disease. Second, it tests the hypothesis that deleting P2Y12 in PSCs protects the NMJ and the motor axon from ALS-induced degeneration. Third, it seeks to identify P2Y12 dependent and independent mechanisms in PSCs and microglia affected with ALS. While some of the experiments in these aims build on each other, each aim is sufficiently independent to stand alone. For example, the experiments in Aim 1 and 3 reveal whether Clopidogrel attenuates ALS pathogenesis by modifying the function of PSCs and spinal cord microglia. Aim 2 provides data regarding the intrinsic role of P2Y12 signaling in PSCs that cannot be attained in vivo using Clopidogrel given its systemic effects. Although a major goal in Aim 3 is to identify downstream signaling of P2Y12 in PSCs, it also reveals for the first time all pathways, including P2Y12 signaling independent mechanisms, dysregulated in PSCs affected with ALS. Aim 3 also reveals P2Y12 effectors altered in microglia with ALS by Clopidogrel. In all three aims, we perform experiments in both female and male mice of the same strain. We use the necessary number of mice to power each experiment and perform the appropriate statistical analysis.

While presenting this technology in another discussion example, this proposal has additional overarching goals. First, it will define the role of a purinergic receptor, P2Y12, in PSC-mediated maintenance and repair of NMJs. Second, it will test the efficacy of a purinergic drug in slowing and reversing ALS pathogenesis. Third, it will identify downstream effectors of P2Y12 in PSCs affected by ALS. We will perform experiments in both female and male mice of the same strain origin within each experiment.

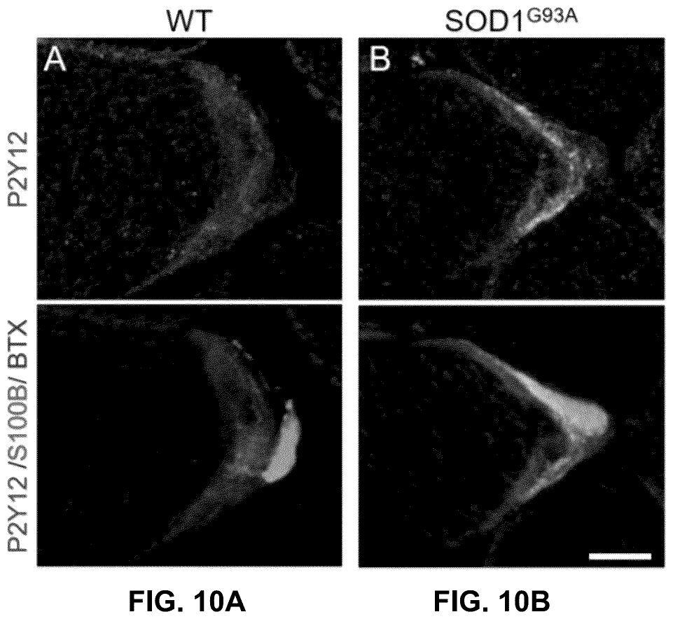

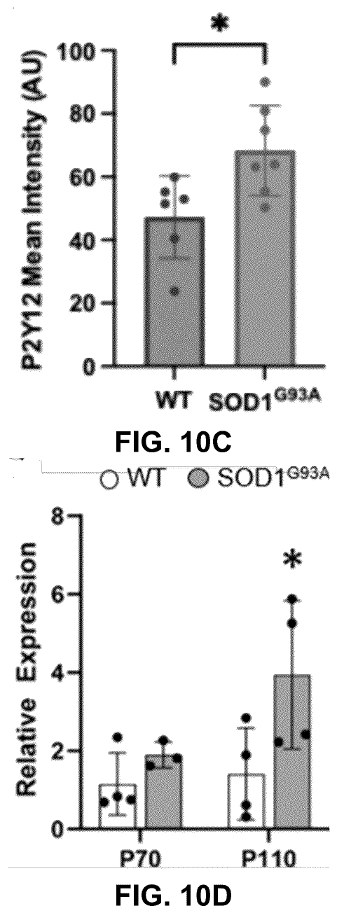

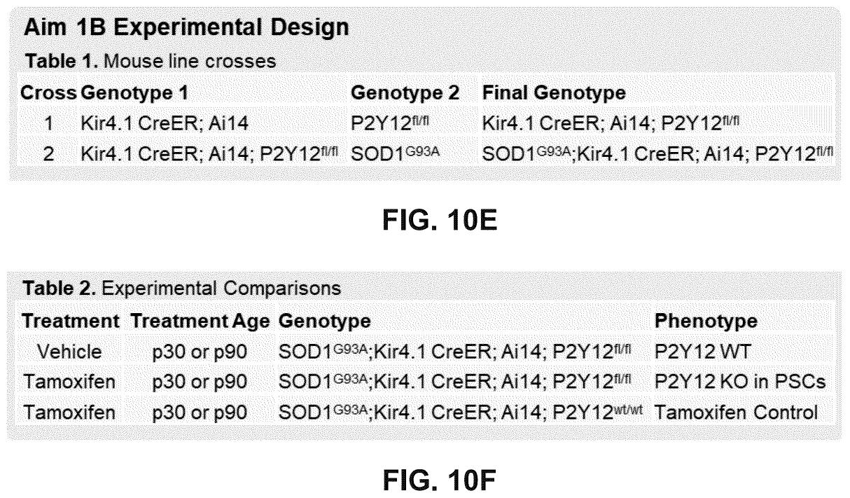

Present Aim 1 (presently disclosed): Test the efficacy of Clopidogrel at slowing the initiation and progression of ALS in live mouse models of the disease. Overview and Supporting Information: Since we had found that P2Y12 is expressed by PSCs at the NMJ (FIG. 7A, FIG. 2B) and upregulated in ALS (e.g., FIG. 10A, FIG. 10B, FIG. 10C, FIG. 10D), we decided to assess the impact of Clopidogrel treatment on NMJ pathology in a mouse model of ALS.

We treated SOD1^{G93A} mice with either vehicle or Clopidogrel (1 mg/kg) for 30 days starting at symptom onset (90 days old). Importantly, this experiment provided exciting initial data indicating that Clopidogrel protects the NMJ in the extensor digitorum longus (EDL) muscle from ALS-induced degeneration in 120-day old SOD1^{G93A} mice (FIG. 3A, FIG. 3B). Clopidogrel treatment also significantly preserved the overall mass of the tibialis anterior (TA) muscle in SOD1^{G93A} mice (see FIG. 3C). The EDL and TA are hindlimb muscles mostly composed of fast-twitch fibers that play similar function in leg movement. These muscles are adjacent to each other and are affected early in ALS38. Based on these findings, we propose that PSCs remain in a homeostatic phenotype in the presence of Clopidogrel to promote the repair of NMJs in ALS (see FIG. 3D).

In addition to preserving NMJs, we wanted to determine whether Clopidogrel has a measurable impact on the mobility of SOD1^{G93A} mice. To this end, before sacrificing mice for histological analysis, we examined the gait patterns of SOD1^{G93A} mice treated with Clopidogrel and vehicle using the DigiGait™ system. Previous studies using DigiGait testing on SOD1^{G93A} mice found that they accumulate gait abnormalities including decreased stride frequency and increased swing duration among other changes.

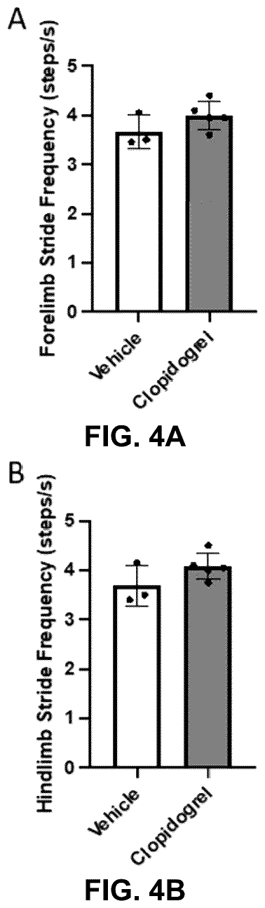

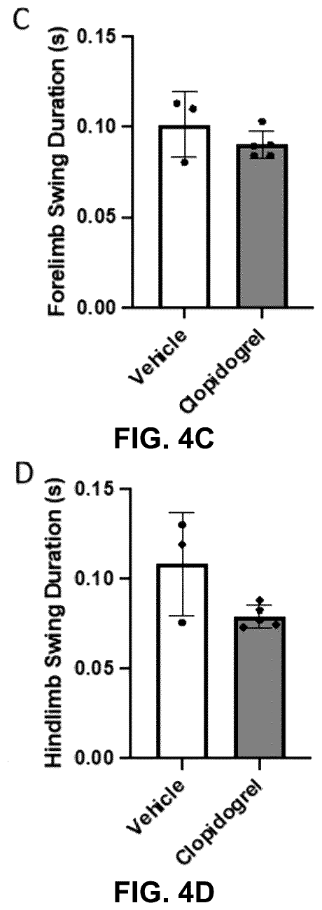

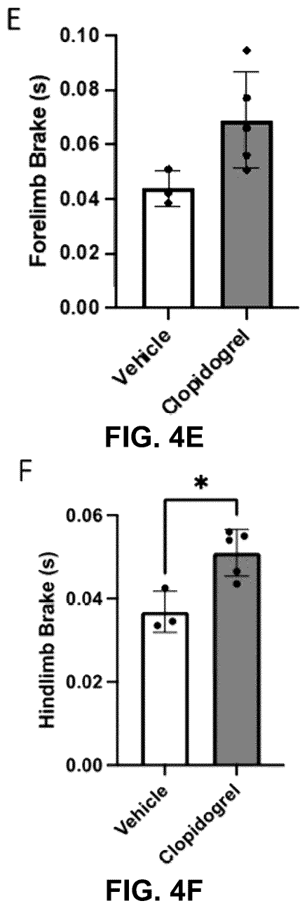

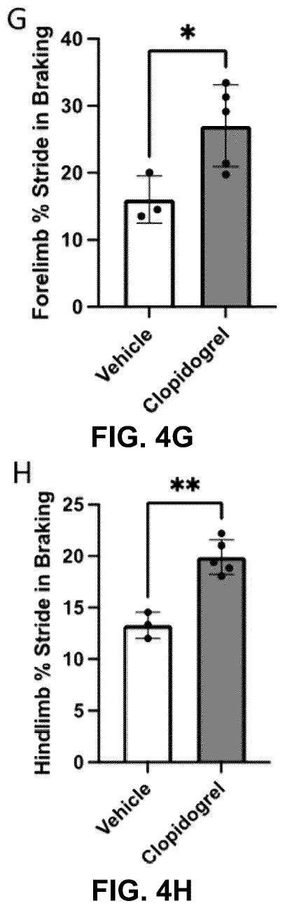

We found that Clopidogrel treatment improved gait parameters of the SOD1^{G93A} mice. Compared to vehicle, Clopidogrel increases stride frequency and reduces swing duration (see FIG. 4A, FIG. 4B, FIG. 4C, and FIG. 4D). Furthermore, Clopidogrel treatment increased the brake time and percent brake of stride (see FIG. 4E, FIG. 4F, FIG. 4G, and FIG. 4H), which is important for force generation. Altogether, these findings indicate that Clopidogrel treatment improves the ability of SOD1^{G93A} mice to coordinate, accelerate and power themselves to complete steps. As such, the treatments claimed herein are enabled.

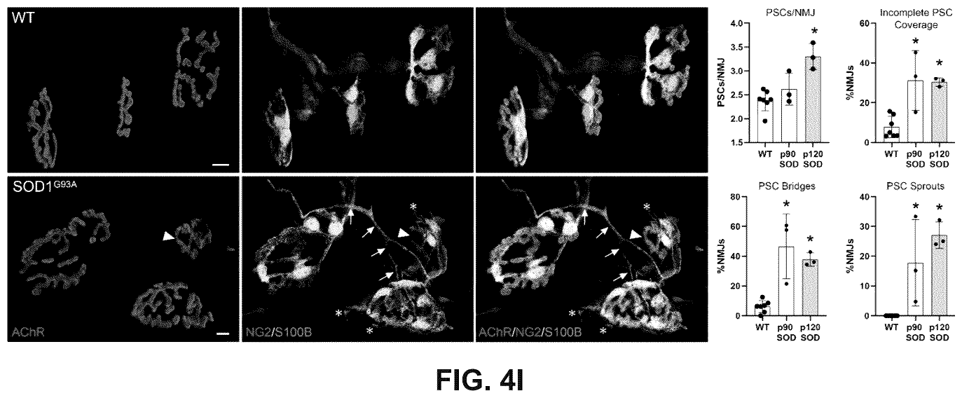

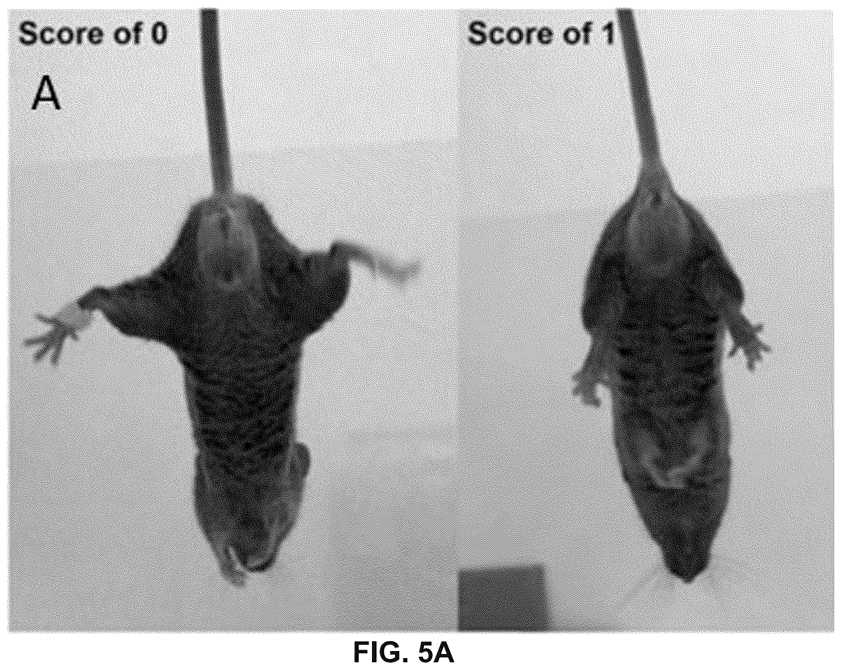

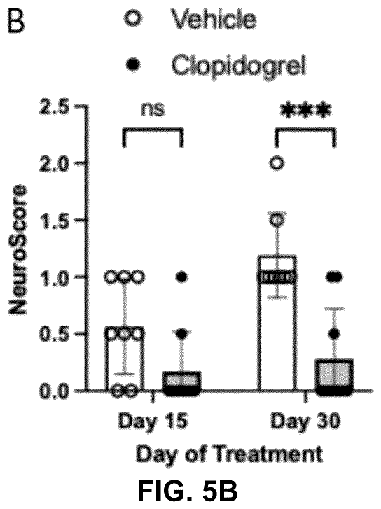

We also assessed disease progression in mice using the neurological scoring system (NeuroScore) which is used to determine disease stage in SOD1^{G93A} mice. NeuroScore involved observing the ability of mice to control the position and mobility of both hindlimbs independently of each other. In this 0-4 point scoring system, 0 represents normal control and 4 represents complete loss of the ability to command hindlimb movement (for example, see FIG. 5A). SOD1^{G93A} mice were examined at 15 and 30 days after treatment with Clopidogrel and Vehicle starting at 90 days of age. NeuroScore revealed that the disease progressed slower in Clopidogrel treated compared to vehicle SOD1^{G93A} mice (FIG. 5B). Together, the NeuroScore, DigiGait and cellular data strongly indicate that Clopidogrel slows the progression of ALS pathogenesis in SOD1^{G93A} mice.

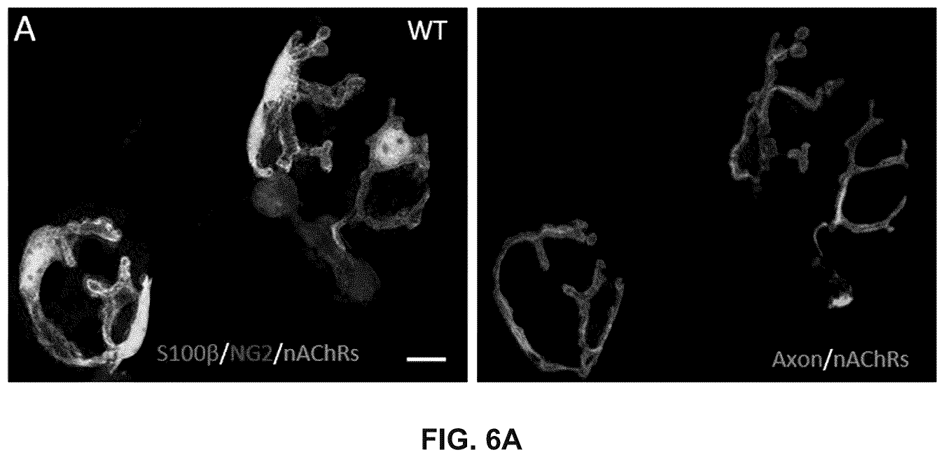

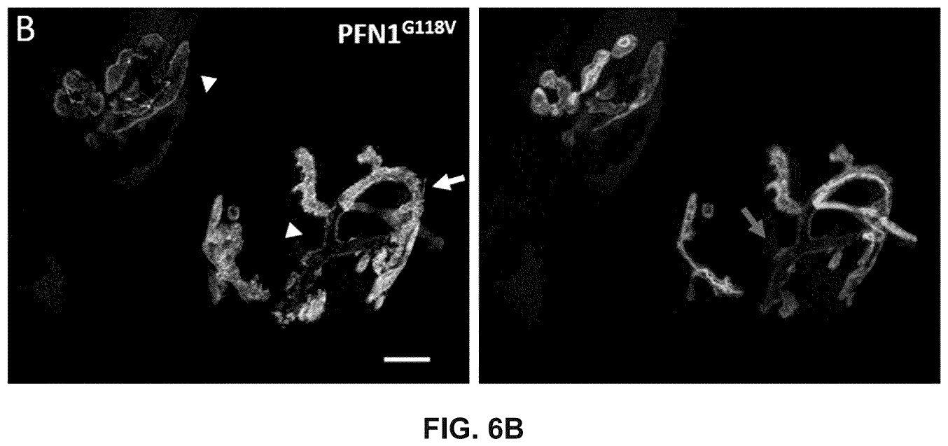

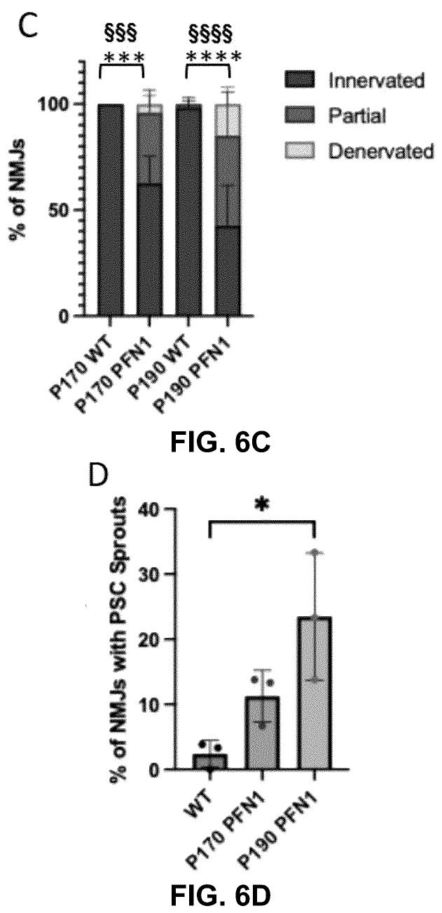

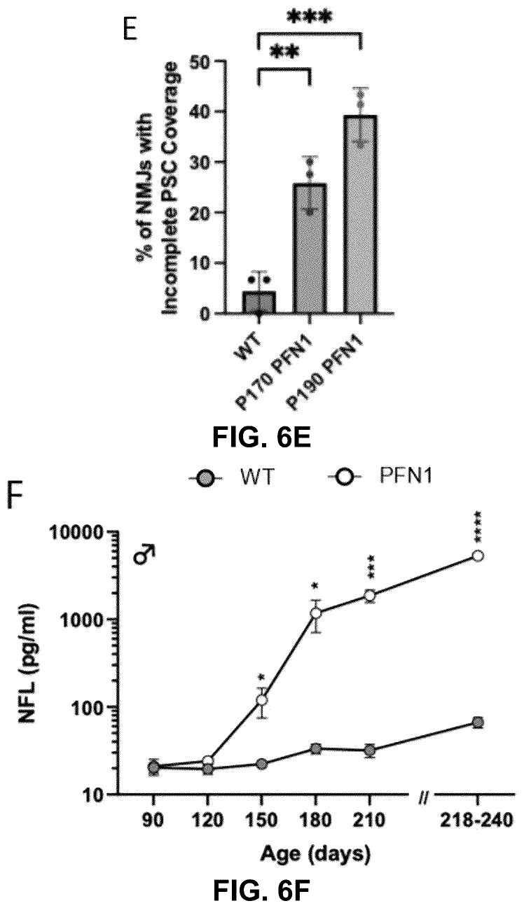

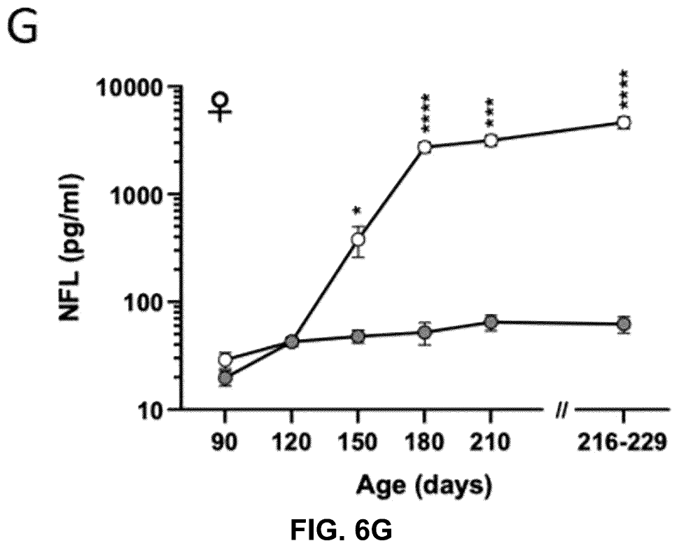

Our goal is to expand on these exciting initial findings by performing additional studies with Clopidogrel using SOD1^{G93A} mice and another mouse model of ALS and PrP-hPFN1^{G118V} (PFN1^{G118V}) The PFN1^{G118V} mouse line is a recently established mouse model of ALS which we chose to examine for the following reasons. (1) PFN1^{G118V} mice display cytoplasmic TDP43 inclusions in motor neurons, which are found in more than 95% of individuals with ALS41, in contrast to SOD1^{G93A} mice42. (2) We have worked recently with ALS TDI (Therapy Discovery Institute) to confirm the suitability of this mouse line for studies to test therapeutic interventions for ALS. We have found that both male and female PFN1^{G118V} mice progressively acquire ALS-related pathology that includes NMJ degeneration (FIG. 6A, FIG. 6B, FIG. 6C).

Additionally, we have found that PSCs fail to cover the NMJ and present with sprouts in PFN1^{G118V} mice (FIG. 6D, FIG. 6E). These mice also progressively present with increased levels of plasma neurofilament light chain (Nf-L), a blood-based biomarker of ALS (FIG. 6F, FIG. 6G). We have found that PFN1^{G118V} mice exhibit ALS-related pathology housed in our facility at Brown University and at ALS TDI. Thus, PFN1^{G118V} mice do not have the issues of other mouse models of ALS that either do not present with obvious ALS pathology or the penetration of the disease varies depending on housing condition. This makes PFN1^{G118V} mice an attractive model to study ALS pathogenesis alongside SOD1^{G93A} mice.

FIG. 6A and FIG. 6B show representative NMJs from P190 WT and PFN1^{G118V} mice with arrow heads showing lack of PSC coverage, white arrow showing a PSC sprout, and orange (or now light grey) arrow showing partial denervation. FIG. 6C shows the innervation status of 30 NMJs from female P135, P170, and P190 WT and PFN1^{G118V} mice was determined. An NMJ was classified as innervated if >95% of the nAChRs was covered with SV2/2H3 staining, partial if 5-95% was covered, and denervated if <5% was covered. Two-way ANOVA ***p<0.001, ****p<0.0001 comparing fully innervated NMJs and § § § p<0.001, § § § § p<0.0001 comparing partially denervated NMJs.

FIG. 6D shows the number % of NMJs with PSC sprouts and FIG. 6E shows incomplete PSC coverage was analyzed over 30 NMJs per mouse. FIG. 6F and FIG. 6G show how neurofilament light chain levels were measured monthly in male (FIG. 6F) and female (FIG. 6G) WT and PFN1^{G118V} mice. Statistical significance was determined using multiple unpaired t-tests with Welch's correction. Significance levels: *p>0.05, ***p<0.001, and ****p<0.0001. Scale bar=10 μm.

Discussion of Research Design (current) and Experimental Methods: Currently Disclosed Aim 1A: Identify the optimal Clopidogrel dose to protect NMJs from ALS. We found that systemic administration of Clopidogrel at 1 mg/kg for 30 days starting at symptom onset reduces NMJ degeneration during the early symptomatic stage of ALS in SOD1^{G93A} mice. To expand on these exciting findings, we test the efficacy of higher doses of Clopidogrel to identify the treatment that optimally protects NMJs and cells of the neuromuscular system from degenerating in SOD1^{G93A} and PFN1^{G118V} mice. We test the safety and efficacy of higher doses of Clopidogrel in ALS model mice because other studies that have repurposed Clopidogrel to treat conditions such as diabetic cardiomyopathy and chronic kidney disease have found that the optimal dosage of Clopidogrel depends heavily on the condition being treated.

We use SOD1^{G93A} and PFN1^{G118V} mice crossed with S100-GFP;NG2-DsRed mice20, in which PSCs are uniquely labeled with GFP and DsRed and all other SCs are labeled with only GFP. We refer to the offspring from these crosses as double labeled (DL) mice (DL-SOD1^{G93A} and DL-PFN1^{G118V}). The presence of fluorescent markers in PSCs and other SCs facilitate visualization in this discussion of a sub-aim as well as in the molecular analysis of PSCs in Aim 3.
Experiment 1A-1: Determine if higher Clopidogrel doses are safe to administer to mouse models of ALS. First, we investigate whether we can safely administer higher doses of Clopidogrel to DL-SOD1^{G93A} and DL PFN1^{G118V} mice. In this pilot study, we administer Clopidogrel via intraperitoneal (IP) injections at 4, 16, 32, and 60 mg/kg. Clopidogrel is administered every day for 15 days starting prior to symptom onset in both DL-SOD1^{G93A} and DL-PFN1^{G118V} mice (P75 DL-SOD1^{G93A} and P135 DL-PFN1^{G118V} mice; see example timeline of disease progression for both mouse models in FIG. 7D).

The range of doses in this experiment has been previously reported in mouse studies. A group of mice treated with vehicle (saline) serve as controls. We use six DL-SOD1^{G93A} and DL-PFN1^{G118V} mice per treatment (Clopidogrel and vehicle) and sex. All mice are weighed daily, and are qualitatively assessed for bleeding, water and food intake, and lethargic behavior. We select two of the four higher doses of Clopidogrel alongside 1 mg/kg to perform additional testing on mouse models of ALS in Experiment 1A-2 below. We choose doses which do not (1) affect body weight, (2) cause bleeding, (3) reduce food or water intake, or (4) result in abnormal behaviors. If none of the doses tested adversely affect mice, we will perform additional studies in Experiment 1A-2 below using Clopidogrel at 1, 32 and 60 mg/kg. This will allow us to test the effect of low, intermediate and high doses of Clopidogrel on ALS pathogenesis.
Experiment 1A-2: Testing the efficacy of higher doses of Clopidogrel on the NMJ, PSCs, motor neurons, and spinal cord microglia with ALS. We have designed this experiment to achieve two goals. First, we will be able to generate additional data to support using 1 mg/kg of Clopidogrel to treat ALS. Second, we will determine whether two higher doses identified in Experiment 1A are even more effective at protecting NMJs, motor neurons and spinal cord microglia from ALS-induced degeneration. Clopidogrel dissolved in saline will be administered prior to symptom onset to both DL-SOD1^{G93A} (at P75) and DL-PFN1^{G118V} (at P135) mice every day for 45 days, which will be sufficient time of exposure to impact ALS pathogenesis. These two treatment regimens will provide critical insight regarding the efficacy of Clopidogrel during the initial (P90 for SOD1^{G93A} and P165 for PFN1^{G118V}) and mid-stage (P120 for SOD1^{G93A} and P180 for PFN1^{G118V}) of the disease in both mouse models of ALS. Mice treated with vehicle (saline) will serve as controls.

To assess NMJ integrity, we will dissect out the extensor digitorum longus (EDL), a hindlimb muscle composed primarily of fast-twitch muscle fibers impacted early and severely in ALS. We will also examine NMJs in the diaphragm, a muscle composed of slow-, intermediate- and fast-twitch muscle fibers in which NMJ degeneration leads to respiratory failure and death of individuals with late-stage ALS. We will visualize NMJs by immunolabeling for neurofilament and synaptic vesicle protein 2 (SV2) which together reveal the motor axon and with fluorescently-tagged α-bungarotoxin which labels muscle postsynaptic nicotinic acetylcholine receptors (nAChRs).

To determine the innervation status of NMJs, we will analyze the overlap of the motor axon terminal with the muscle postsynaptic region. We will bin NMJs as fully innervated, partially innervated, or fully denervated based on the overlap between the axon and muscle postsynaptic region. We will also analyze the fragmentation of postsynaptic nAChRs, which increases in later stages of ALS. PSCs will be identified by the co-expression of GFP and DsRed as well as their location at the NMJ. We will count the number of PSCs at each NMJ. We will also measure PSC coverage of the NMJ and whether they present with sprouts as done previously by our lab.

To assess motor neuron health, we count the number of motor neurons in 30 μm coronal sections collected from the lumbar and cervical regions of the spinal cord. We will use antibodies against the neuronal nuclei protein (NeuN) and vesicular acetylcholine transferase (VAChT) to identify motor neurons. This method has been used in published studies by our lab. We will also immunolabel for anti-cleaved caspase 3 to assess the number of dying motor neurons. In addition, we will examine the impact of Clopidogrel treatment on spinal cord microglia. To analyze the phenotype of microglia, we will immunolabel for ionized calcium binding adaptor molecule 1 (Iba1), a microglia marker. Microglia with small cell bodies and long branched processes will be deemed to be in a homeostatic state. Microglia with large cell bodies, phagocytic cups and short processes will be deemed to be in a hyperactive state. We will also assess whether Clopidogrel differentially affects microglia near and associated with motor neurons in ALS as done previously by our lab.

During the course of treatment, we collect blood samples to examine levels of neurofilament light chain (Nf-L), a biomarker of neuronal degeneration. Nf-L is routinely used as a biomarker to assess ALS disease progression in mice and humans. To determine if Clopidogrel prevents Nf-L from increasing in the blood and thus protects neurons from ALS, blood plasma will be collected at the start and the end of treatments. Nf-L in plasma will be analyzed using ultrasensitive SIMOA assay from the Quanterix and Simoa Platform located in the Biomarkers Core Facility at Brown University.

Through this work, we identify the optimal dose of Clopidogrel to use in Aim 1B based on which dose has the most significant benefit on preserving: (1) the structural integrity of NMJs in both the EDL and diaphragm muscles, including its effect on PSCs; (2) the health of motor neurons and glial cells in the spinal cord; and (3) normal levels of Nf-L in blood plasma.

Sampling & Statistics: Assignment of different Clopidogrel doses and vehicle treatments will be randomized. All data will be blinded. Images of NMJs, PSCs, motor neurons, and microglia will be randomly sampled using the fractionator method59. Images will be obtained with a Zeiss 900 Airyscan confocal microscope using 20×, 0.8 numerical aperture (NA) and 63×, 1.4 NA objectives. We will blind data during acquisition and analysis to avoid bias. We will use eight DL-SOD1^{G93A} and DL-PFN1^{G118V} mice per treatment and sex for these cellular analyses. Separate statistical comparisons will be made for DL-SOD1^{G93A} and DL-PFN1^{G118V} mice using two-way ANOVA.
Aim 1B: Determining the impact of Clopidogrel on muscle function, mobility and lifespan of two mouse models of ALS. We determine whether Clopidogrel treatment preserves motor function, mobility and lifespan in DL-SOD1^{G93A} and DL-PFN1^{G118V} mice. This allows us to evaluate the extent to which any improvements in NMJ, motor neuron, and glial health found in Aim 1A translate into functional improvements. Clopidogrel treatment will begin prior to symptom onset. By starting treatment prior to symptom onset, we will be able to determine the effectiveness of Clopidogrel at both preventing and in slowing symptom progression of ALS induced pathogenesis. We perform electromyography to measure compound muscle action potential (CMAP) and behavioral tests to assess muscle strength and coordination, as well as determine the lifespan of mice.
Experiment 1B-1: Examining the effect of long-term Clopidogrel treatments on muscle function and mobility of SOD1^{G93A} and PFN1^{G118V} mice. We will administer the optimal dose of Clopidogrel identified in Aim 1A daily starting prior to symptom onset and continuing until their humane endpoint (see SOD1^{G93A} and PFN1^{G118V} symptom timeline in FIG. 7D). In SOD1^{G93A} mice, treatment will begin at 75 days of age. In PFN1^{G118V} mice, treatment will begin at 135 days of age.

By starting treatments prior to symptom onset, we will generate critical data regarding the efficacy of Clopidogrel at preventing and slowing the progression of ALS-associated pathology. We will evaluate body weight, motor function by measuring CMAP, and behavioral ability throughout the course of treatments. Electromyography will be performed to measure CMAP from the right TA muscle on a monthly basis. This frequency of testing will allow the mice to adequately recover from the previous minor injury caused by temporarily inserting electrodes to record CMAP. Mice will be anesthetized and placed on a heating pad, with the locations for electrode insertion shaved and cleaned. Active and passive stimulating electrodes (A-M system, USA) will be inserted in the sciatic notch and at the base of the tail, respectively.

Monopolar Teflon-coated needle electrodes for recording EMG signal (A-M system, USA) will be inserted into the belly of the TA and the Achilles tendon of the same leg, with a grounding electrode placed on the ear of the mouse. We will use a micro-current stimulation device (Master-9, A.M.P.I, Israel) and a flexible stimulus isolator (Iso-Flex, A.M.P.I, Israel) to stimulate the sciatic nerve in single pulses beginning at an intensity of 2.75 mA. Intensity of the stimulus will gradually increase until the amplitude of the muscle response no longer increases. The intensity of stimulation will then increase one last time to produce a myograph corresponding to the supramax intensity. The amplitude of the response to this stimulation will be recorded by a data acquisition system (Bio-Signal Technologies, USA). Spike2 software (Cambridge Electronic Design Limited, UK) will be used to analyze traces, with CMAP measured from the peak of the positive deflection to the peak of the negative deflection. Mean CMAP amplitude will be calculated from 5-6 traces.

We will examine mice for neuropathology using the NeuroScore system as described in Aim 1A. We will expand our DigiGait analysis described in Aim 1A to also include information on paw position and angle, stride length, speed, fore- and hind-paw base width, and paw overlap, as previously described in motor function evaluations of rodent ALS models. In addition, we plan to perform rotarod, and wire hang tests which assess other types of motor function in these mice such as strength and coordination.

We will test motor function once per month and stop testing mice once they are unable to perform adequately on a rotarod and lose more than 20% of their adult weight within one week. Experiment 1B-2: Determine the long-term efficacy of Clopidogrel on reducing neurodegeneration and extending the lifespan of SOD1^{G93A} and PFN1^{G118V} mice. We will determine the impact of long-term Clopidogrel treatment on neurodegeneration and lifespan. We will perform blood draws to examine Nf-L levels, a biomarker of neurodegeneration, prior to and every month after commencing treatment.

We will track the mice to determine their lifespan. We will use the humane endpoints to compare the survival of Clopidogrel and vehicle treated mice. Humane endpoint will be defined as functional paralysis of both hindlimbs and loss of righting reflex within 20 seconds of being placed in lateral recumbency, as well as exhibiting a NeuroScore of 4 as defined in Hatzipetros, et al. Sampling & Statistics: Clopidogrel and vehicle treatments will be randomized. We will treat 24 mice per genotype and sex with Clopidogrel or vehicle. SOD1^{G93A} and PFN1^{G118V} mice will be analyzed separately using two-way ANOVA to evaluate the effects of treatment on muscle function, mobility and lifespan.

Expected outcomes, alternative approaches, and future directions: For Aim 1A, the primary limitation is discovering the optimal dose of Clopidogrel that protects the NMJ and extends the lifespan of mouse models of ALS. The dose of Clopidogrel at which it is most efficacious for a given disease ranges from 1 mg/kg to 60 mg/kg in mice. While the doses we will test cover this range, it is plausible that a different Clopidogrel dose outside of this range is most effective at treating ALS pathogenesis in the mouse models of the disease. However, we view this as a minor limitation given that we already have found that 1 mg/kg Clopidogrel treatment protects NMJs in ALS.

We would also have the ability to test other doses based on information we gather during the course of this study. For instance, we would test a range of dose closer to 1 mg/kg if we find that 4 mg/kg is harmful to the mouse models of ALS. While the premise of this proposal is that Clopidogrel protects NMJs in ALS by blocking P2Y12 in PSCs, we recognize that other cells expressing P2Y12 may also contribute to the positive effects of Clopidogrel on NMJs. For instance, the beneficial effects may partially or fully emanate from blocking P2Y12 in microglia and/or platelets. Our analysis of microglia phenotype in Aim 1A and molecular composition in Aim 3 will provide insights about the effect of Clopidogrel on microglia in ALS. If we find that Clopidogrel has minimal effect on the morphology (Aim 1A) and molecular composition (Aim 3) of PSCs and microglia with ALS, we will assess the effect of Clopidogrel on platelets and other cells, such as macrophages and mast cells, in ALS.

Platelets are a particularly attractive cell type because Clopidogrel has been shown to reduce levels of pro-inflammatory genes in these cells. Additionally, platelets' generation of serotonin has been shown to decrease in ALS and to closely correlate with survival in humans with the disease. Regardless, the data accrued in this study will provide leads to target P2Y12 to treat ALS using an already FDA-approved drug, Clopidogrel/Plavix, and potentially other known and tested P2Y12 inhibitors. In Aim 1B, we do not expect to encounter any major issues in assessing Clopidogrel effect on motor function and lifespan of the mouse models of ALS. In future studies, we will assess the impact of commencing Clopidogrel treatments after the onset of ALS-pathogenesis. We will also design experiments to test whether administering Clopidogrel together with Riluzole and other drugs shown to slow ALS, albeit minimally, through different pathways provides even more protection against ALS.

As this work has taken years to develop is now provided Currently Disclosed Aim 2: Define the direct role of P2Y12 in PSC-mediated maintenance and repair of NMJs in ALS. Overview and Supporting Information: Despite their important roles at NMJs, it remains unknown whether PSCs present with cellular and functional abnormalities prior to NMJs degenerating in ALS. Answering this question is important for determining whether PSCs are drivers of NMJ degeneration in ALS or simply innocent bystanders impacted by deleterious changes in other cells, such as motor neurons and muscles.

To answer this question, we examined PSCs in the EDL muscle of DL-SOD1^{G93A} mice at 90 days of age (P90), when most NMJs are still intact, and also at P120, when a significant number of NMJs have degenerated. Our initial analysis revealed that abnormalities in PSC morphology precede significant degeneration of NMJs (FIG. 4I, FIG. 8B, FIG. 8C). We found significantly more PSCs with sprouts in P90 DL-SOD1^{G93A} mice (FIG. 8B). We also found that PSCs cover less of the NMJ in P90 DL-SOD1^{G93A} mice (FIG. 8C). Incomplete PSC coverage of the NMJ likely impacts PSC modulation of neurotransmission and disrupts the cellular barrier which protects the NMJ from external and potentially harmful stimuli. We found that PSCs undergo the same deleterious morphological changes in PFN1^{G118V} mice (FIG. 6D, FIG. 6E), further indicating that abnormalities in PSCs contribute to NMJ degeneration in ALS.

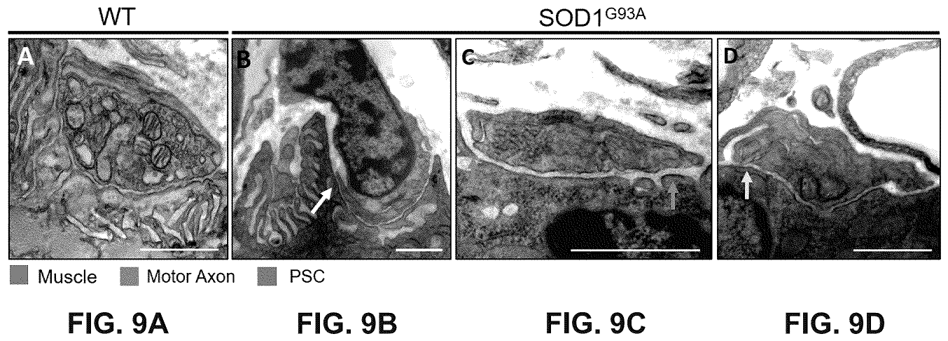

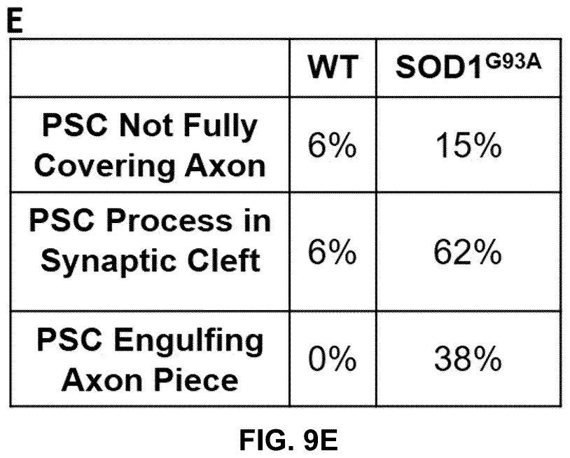

We next used transmission electron microscopy (TEM) to discern the relationship between PSCs and the NMJ in ALS at a higher resolution. We examined NMJs again in the EDL muscle of P90 of control and SOD1^{G93A} mice. In control mice, the majority of PSCs completely capped the presynapse (FIG. 9A). In contrast, PSCs had several morphological abnormalities in SOD1^{G93A} mice. There were more SOD1^{G93A} NMJs with PSCs only partially capping the presynapse and with processes intruding into the synaptic cleft, a change in PSCs shown to impair neurotransmission and contribute to NMJ degeneration (FIG. 9B, FIG. 9C). SOD1^{G93A} PSCs also appeared to be in the act of engulfing segments of the presynapse (FIG. 3D), suggesting that PSCs may be more phagocytic in ALS.

The TEM data showing PSC abnormalities at otherwise healthy, innervated NMJs support our light microscopy finding (FIG. 6D, FIG. 6E, FIG. 8B, FIG. 8C) that PSCs alter their morphology prior to motor neuron degeneration in ways that are detrimental to the stability of the NMJ. Altogether, these data indicate that PSCs transition to a reactive phenotype in the early stages of ALS progression that may contribute to NMJ degeneration. Thus, molecular mechanisms that mediate the transition of PSCs from a homeostatic to a reactive state could be therapeutic targets to preserve the NMJ in ALS. Purinergic signaling has been shown to guide the transition of PSCs between homeostatic and reactive modes in healthy and more acutely in ALS-afflicted NMJs.

Because of these findings, we sought to identify the purinergic receptors enriched in PSCs to examine their roles in ALS. We discovered P2Y12 enriched in PSCs20 (FIG. 2A, FIG. 7A, FIG. 2B). There are additional reasons for why P2Y12 is an attractive candidate for mediating the function of PSCs in ALS. These include: (1) P2Y12 has been shown to mediate inflammation and the synaptic pruning activity of microglia23. (2) Nothing is known about P2Y12 function in mediating the activity of PSCs in normal and under stress conditions that includes in ALS. (3) Targeting this receptor using Clopidogrel, see Aim 1, protects the NMJ from degeneration in SOD1^{G93A} mice. (4) We found P2Y12 protein significantly increased at NMJs of early symptomatic SOD1^{G93A} mice (FIGS. 10A-10C). Transcriptional analysis also shows P2Y12 starting to increase in the presymptomatic and becoming significantly elevated in the early symptomatic phase of ALS pathogenesis in SOD1^{G93A} mice (FIG. 10D). Because of these findings, we propose to test the direct role of P2Y12 in PSC-mediated repair of NMJs with ALS using SOD1^{G93A} mice in this Aim.

FIG. 10A, FIG. 10B, and FIG. 10C show IHC visualization and analysis showing P2Y12 (red in the merged image, or different shade of grey) increased at NMJs of the TA muscle of SOD1^{G93A} compared to WT mice at P110. (D) qPCR evaluation of P2Y12 mRNA levels in pre symptomatic (P70) and symptomatic (P110) SOD1^{G93A} TA muscles. *p<0.05 versus age-matched control; n=7 for IHC and 3-4 for qPCR. Scale bar=5 μm.

Disclosure of Research Design (current) and Experimental Methods: Aim 2A: Determine the direct contribution of P2Y12 in PSCs to ALS-induced NMJ degeneration. We will delete from PSCs in SOD1^{G93A} mice. For this, we have created a conditional P2Y12fl/fl;Sox10 Cre mouse line in which the Sox10 promoter, active in all SCs, drives P2Y12 receptor deletion in both PSCs and other SCs. We will breed this line with SOD1^{G93A} mice and with a Rosa-stp-tdTomato (tdTom) transgenic line to generate SOD1^{G93A};Sox10-Cre;P2Y12fl/fl;tdTom mice. The presence of tdTomato will allow us to clearly visualize PSCs and ascertain the activity of constitutive Cre. We will use SOD1^{G93A};Sox10-Cre;tdTom, Sox10-Cre;P2Y12fl/fl;tdTom, and Sox10-Cre;tdTom as controls. We choose to use Sox10-Cre for two reasons. One, our data shows that P2Y12 is expressed primarily in PSCs in skeletal muscles (FIG. 2A, FIG. 7A, FIG. 2B). Second, this approach will allow us to examine the impact of deleting P2Y12 in all PSCs on muscle function, mobility and lifespan of SOD1^{G93A} mice. It is worth noting that we are not concerned about these mice prematurely dying nor presenting with obvious neuropathology based on prior studies of P2Y12 global knockout mice.

We will determine the impact of deleting P2Y12 in PSC-mediated NMJ degeneration in ALS at P70, P90, P120 and P140 using the aforementioned experimental and control animals. FIG. 7D depicts the correspondence between the age of SOD1^{G93A} mice and disease progression. To glean as much information as possible from these experiments, we will examine NMJs in both the EDL and diaphragm muscles. The progression of ALS pathology is delayed in the diaphragm relative to the EDL muscle in SOD1^{G93A} mice as the disease follows a caudal to rostral progression in these mice. The two muscles also differ in their anatomical locations, functional demands, and muscle fiber type composition. Hence, comparing these two different muscles provide opportunities to more globally assess the roles of PSC-enriched purinergic receptors on NMJs in ALS.

In addition to analyzing NMJs and associated PSCs, we will examine spinal motor neurons and resident microglia. This will allow us to determine the extent to which deletion of P2Y12 in PSCs impacts the health of NMJs and motor neurons. We will perform these cellular analyses using the methods described in Aim 1, with the exception that PSCs will be identified in this Aim based on their expression of only tdTomato and location at the NMJ in addition to their unique morphology compared to myelinating SCs. We will also compare levels of Nf-L in the blood between SOD1^{G93A} with and without P2Y12 in PSCs as described in Aim 1.

Sampling & Statistics: Samples will be blinded, imaged and randomly sampled according to the methods described in Aim 1. Female and male mice will be used. We will use six mice per genotype, age and sex for these experiments. Statistical comparisons will be made between SOD1^{G93A};Sox10-Cre;P2Y12fl/fl;tdTom and control mice that include SOD1^{G93A};Sox10-Cre;tdTom, Sox10-Cre;P2Y12fl/fl;tdTom, and Sox10-Cre;tdTom. A two-way ANOVA will be used to evaluate the effects of genotype, age of treatment, and interactions between genotype and treatment age. Aim 2B: Compare motor function and lifespan of SOD1^{G93A} mice with and without P2Y12. We will examine the effect of deleting P2Y12 in PSCs on motor function, muscle function and lifespan of SOD1^{G93A} mice. Mice will be monitored daily for water and food intake and weekly for body weight.
Experiment 2B-1: We will evaluate motor behavior on a weekly basis starting at P70. This will include DigiGait to collect information on paw position and angle, stride length, speed, fore- and hind paw base width, and paw overlap, as previously described in motor function evaluations of rodent ALS models. In addition, we will perform hindlimb clasping reflex, rotarod, and wire hang tests. We will stop testing mice for motor function once they are unable to perform adequately on a rotarod and lose significant weight. Data analyses will be performed as described in Aim 1.
Experiment 2B-2: We will examine muscle function by measuring CMAP as also described in Aim 1. Experiment 2B-3: We will track mice to determine their lifespan. We will use the humane endpoints determine the lifespan of SOD1^{G93A};Sox10-Cre;P2Y12fl/fl;tdTom and control mice that include SOD1^{G93A};Sox10-Cre;tdTom, Sox10-Cre;P2Y12fl/fl;tdTom, and Sox10-Cre;tdTom. Humane endpoint will be defined as functional paralysis of both hindlimbs and loss of righting reflex within 20 seconds of being placed in lateral recumbency, as well as exhibiting a NeuroScore of four.

Sampling & Statistics: We will examine 20 mice per genotype and sex for motor function, muscle function and survival analysis. The experimenter will be blinded to genotype and sex. For data analysis, we will use two-tailed unpaired t-test to assess significance in normally distributed data. If assumptions of normality cannot be established, we will use a Mann-Whitney test. To evaluate drug combinations, ordinary one-way ANOVA with Bonferroni post hoc or Kruskal-Wallis ANOVA will be used. Expected outcomes, alternative approaches, and future directions: We predict that deletion of P2Y12 in PSCs will allow them to better respond to damages at NMJs caused by ALS. However, it is possible that deletion of P2Y12 will instead accelerate NMJ degeneration. This latter is an unlikely outcome because our initial analysis using Clopidogrel, a P2Y12 antagonist, shows preservation of NMJs in SOD1^{G93A} mice (FIGS. 3A-3D).

Given that animals completely lacking P2Y12 do not present with obvious abnormalities, we do not expect that deleting P2Y12 from all SCs in the embryo to affect the development of PSCs and the NMJ. Overall, we are confident that the genetic approach outlined in this Aim along with pharmacologically targeting P2Y12 in Aim 1 will provide the necessary data to make additional inferences about the role of this receptor in mediating the actions of PSCs at NMJs with ALS.

Our lab routinely performs the molecular, histological and behavioral analyses described here. All mouse lines proposed in this experiment are maintained as colonies in our lab. We are therefore confident that we will successfully complete this work.

Aim 3: Identify downstream effectors of P2Y12 in PSCs and microglia affected with ALS. Overview and Supporting Information: Purinergic signaling has been shown to modulate intracellular calcium responses to neurotransmission as well as expression of cytokines and growth factors in PSCs and microglia. It also regulates other signaling pathways, such as PI3K, that in turn affect levels of purinergic receptors. Despite these advances, nothing is known about the molecular pathways P2Y12 recruits to influence the function of glial cells. In this aim, we will perform bulk RNA-seq of FACS isolated PSCs to identify molecular pathways independent of and dependent on P2Y12 signaling that disrupt the homeostatic functions of PSC in ALS. We will also identify molecular pathways downstream of P2Y12 signaling in FACS isolated microglia with ALS.

Additional Discussion of Research Design and Experimental Methods: Aim 3A: Perform bulk RNAseq analyses on PSCs and microglia isolated from ALS mice treated with Clopidogrel or vehicle. PSCs will be FACS isolated from the TA and EDL muscles of S100B-GFP;NG2-DsRed (DL) SOD1^{G93A}, DL-PFN1^{G118V} and DL control mice following our published method. Microglia will also be obtained via FACS from the lumbar and sacral regions of the spinal cord following our published protocol. To label microglia for FACS isolation, we will cross the CX3CR1-GFP line, in which microglia are transgenically labeled with GFP88, with SOD1^{G93A} and PFN1^{G118V} mice. This crossing will allow us to FACS isolate microglia from CX3CR1-GFP;SOD1^{G93A} and CX3CR1-GFP;PFN1^{G118V} mice. Mice will be treated for 45 days with either vehicle or Clopidogrel at 1 mg/kg prior to symptom onset (P75 for SOD1^{G93A} and P135 for PFN1^{G118V} mice). PSCs and microglia will be FACS-isolated at the onset of symptoms (P90 for DL-SOD1^{G93A} and P165 for DL-PFN1^{G118V}) and at the symptomatic (P120 for DL-SOD1^{G93A} and P180 for DL-PFN1^{G118V}) phase of ALS-pathogenesis. We will isolate PSCs and microglia from untreated presymptomic mice (P75 for DL-SOD1^{G93A} and P135 for DL-PFN1^{G118V}). We will isolate PSCs and microglia from eight mice per genotype and sex.

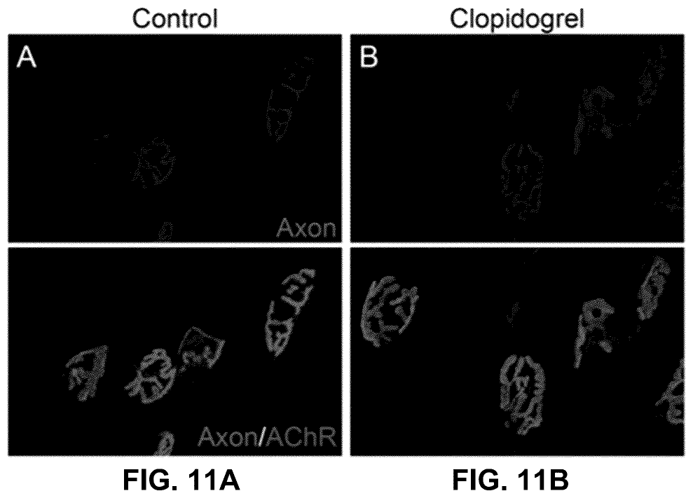

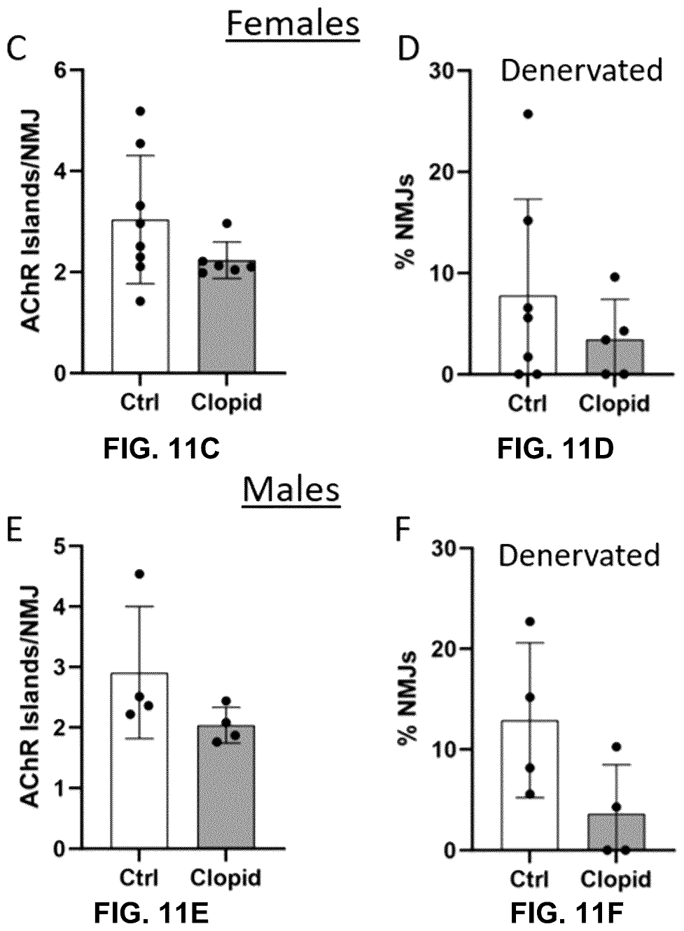

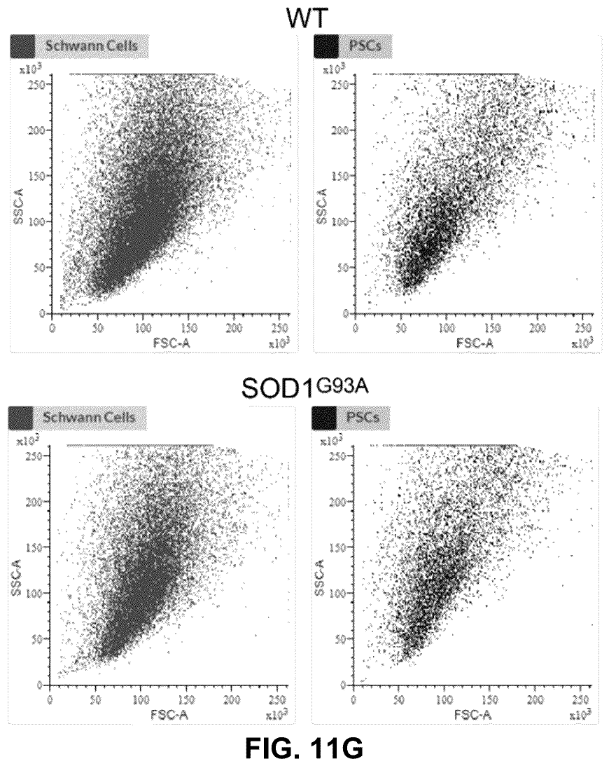

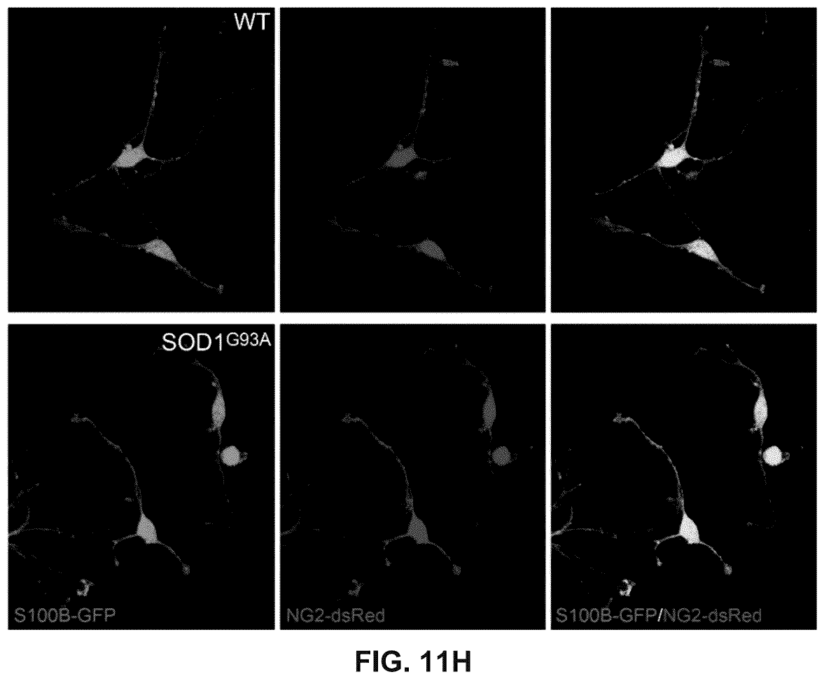

Given our expertise isolating both PSCs and microglia from young and aged mice, we do not foresee issues in obtaining sufficient cells for bulk RNAseq. As proof of principle, we have FACS isolated PSCs from symptomatic DL-SOD1^{G93A} mice. As expected, demonstrating that these cell types maintain expression of their respective markers during the dissociation process (FIG. 11G, FIG. 11H). To compare gene expression across ages and treatments, we will apply our recently published data curation and statistical parameters for both PSCs and microglia. We use 100 transcripts and P-adjusted value of 0.05 as cut-offs to identify differentially expressed genes. Overall, these comparisons will reveal genes and pathways altered in PSCs as well as microglia with ALS treated with vehicle and Clopidogrel before, at the onset and during the progression of ALS pathogenesis. FIG. 11G shows representative FACS scatter plots showing single-labeled S100B-GFP+ SCs (green) and double labeled S100B-GFP+; NG2-dsRed+ PSCs FACS-isolated from P90 WT and SOD1^{G93A} S100B-GFP;NG2-dsRed mice. FIG. 11H shows images of FACS-isolated PSCs in cell culture show that PSCs collected from SOD1^{G93A} S100B-GFP;NG2-dsRed have robust expression of both GFP and dsRed.

Aim 3B: Identify and validate P2Y12 dependent and independent candidate genes altered in ALS-affected PSCs and microglia. We will identify genes of interest altered in PSCs and microglia with ALS that are dependent on and independent of P2Y12 signaling based on the following criteria: 1) Expression is altered in both SOD1^{G93A} and PFN1^{G118V} mice compared to controls; 2) The magnitude of change increases from presymptomatic the to the symptomatic stage; 3)

Blocking P2Y12 prevents the change in expression induced by both the SOD1^{G93A} and PFN1^{G118V} transgenes, which would suggest that Clopidogrel alters the functions of PSCs and microglia through such other genes and pathways. We will stratify for genes with known or predicted functions in synaptic maintenance and repair. These will include genes encoding cytoskeletal molecules, scaffolding proteins, secreted factors, cytokines and other pro-inflammatory molecules as well as receptors that we and others have previously identified in PSCs and microglia. In addition to genes and pathways regulated by P2Y12 signaling, this approach will reveal other genes and signaling pathways dysregulated in PSCs and microglia with ALS that may synergize with P2Y12 signaling to affect the initiation and progression of ALS.

We will use qPCR to validate expression changes of candidate genes in FACS-isolated PSCs and microglia according to methods previously published by our lab. In addition, RNAscope and IHC will be used to validate RNAseq findings. For PSCs, validation experiments will be performed in the TA and diaphragm muscles of SOD1^{G93A}, PFN1^{G118V} and control mice treated with Clopidogrel and vehicle. Validated genes will be further examined in PSCs of SOD1^{G93A};Sox10-Cre;P2Y12fl/fl;tdTom mice, which lack P2Y12 in PSCs and thus should present with the same expression pattern as PSCs treated with Clopidogrel. For microglia, validation experiments will be performed in the lumbar region of the spinal cord of SOD1^{G93A}, PFN1^{G118V} and control mice treated with Clopidogrel and vehicle.

These experiments will reveal candidate genes P2Y12 uses to mediate the actions of PSCs in ALS. We will validate promising candidate genes in human tissue. We will also design future studies to examine the function of candidate genes validated in humans afflicted with ALS.

Expected outcomes: We expect to gain additional mechanistic insights into how Clopidogrel modifies the function of PSCs and microglia to attenuate the initiation and progression of ALS pathogenesis using bulk RNA-seq. We have shown that PSCs and spinal cord microglia under chronic stress increase expression of pro-inflammatory and phagocytic genes among other genes known or suspected of destabilizing synapses and impairing cellular health. We expect Clopidogrel treatment to reduce levels of such genes while maintaining or increasing levels of genes important for the homeostatic function of glial cells.

We make this prediction based on our cellular and functional data in Aim 1 showing the protective effects of Clopidogrel on SOD1^{G93A} mice and its known effect on platelets under stress. It is, however, possible that PSCs in different muscle fiber types (ex.: intermediate- vs fast-twitch fibers which makeup the TA and EDL) and microglia in different regions of the spinal cord (ex.: in the ventral horn and next to motor neurons vs in the dorsal horn) respond in unique ways to Clopidogrel. Our cellular analysis of both cell types in Aim 1 and validation experiments with IHC and RNAscope will largely address this possibility. We don't expect this to be case because our IHC analysis shows P2Y12 in all NMJs and at similar levels and others have shown this receptor expressed in all microglia. Our temporal analyses of both PSCs and microglia with and without Clopidogrel should provide leads about genes and pathways dysregulated prior to ALS pathogenesis.

This information is arguably more critical for PSCs given that a comprehensive analysis of their molecular landscape in ALS has not been performed. Given our findings that PSCs begin to accrue cellular abnormalities prior to NMJ degeneration in ALS, it is even more crucial to identity genes and pathways that cause PSCs to lose their homeostatic phenotype. Our analyses will provide a similar temporal view of the molecular landscape of spinal cord microglia in ALS. Overall, we don't expect to encounter any issues performing these analyses on both PSCs and microglia. This is because we have already performed these analyses in both cell types from aged mice, in which muscles and the spinal cord at a minimum show similar pathology as in the early stage of ALS. Moreover, our data shows that both cells maintain expression of their respective fluorescence proteins during the progression of ALS symptoms.

Timeline and Summary: This work has taken years and will take additional years to complete with the purpose of improving human lives. In the first 3 years, we define the role of P2Y12 signaling in PSC-mediated maintenance and repair of NMJs in ALS. This includes testing the impact of inhibiting P2Y12 activity both pharmacologically in Aim 1, using the P2Y12 antagonist Clopidogrel, and genetically in Aim 2 using conditional mice in slowing and preventing the accumulation of ALS-related pathogenesis. In parallel, we examine microglia, particularly in Aim 1 to determine whether Clopidogrel positive effects are carried out by microglia. In the following 2 years, we perform analysis of motor function and determine the lifespan of ALS mice with and without altered purinergic signaling related to Aims 1 and 2. We also identify and validate molecular mechanisms dependent and independent on P2Y12 signaling dysregulated in PSCs and microglia affected with ALS in Aim 3. Overall, the work resulting from this study could lead to critical preclinical data to advance a new treatment for ALS. It also provides important basic cellular and molecular information for future studies to continue to make strides to fully cure ALS. This will be possible because we have the expertise and additional resources required to complete all the experiments proposed in this study successfully regardless of outcome during the funding period.

In another example of how many years the technology disclosed herein and all the work has taken, we also discuss Previous AIM 1 (previous AIM): Defining the role of P2Y12 in PSC-mediated maintenance and repair of NMJs in ALS. OVERVIEW: The functional state of PSCs was thought to be guided by synaptic activity[[15]]. Therefore, disruptions in synaptic transmission associated with ALS may profoundly influence whether PSCs participate in synaptic maintenance versus repair activities. We have gathered strong data indicating that PSCs become reactive early, prior to denervation, in ALS. We assessed the morphology of PSCs in the extensor digitorum longus (EDL) of male SOD1^{G93A} mice at the onset (postnatal day 90 (P90)) and mid symptomatic (P120) stage. To facilitate analysis of PSCs, we crossed SOD1^{G93A} mice with the S100B-GFP;NG2-dsRed line to generate S100BGFP;NG2-dsRed;SOD1^{G93A} mice. In these mice, PSCs are marked by the co-expression of GFP and dsRed (FIG. 2A and FIG. 4I).

The NMJ postsynaptic region was visualized by staining for nicotinic acetylcholine receptors (AChRs) using fluorescently tagged alpha-bungarotoxin (fBTX). Our initial analysis shows that the number of PSCs per NMJ increases with the progression of ALS-pathogenesis in SOD1^{G93A} compared to control mice (FIG. 4I). Despite this increase in number, PSCs cover less of the NMJ at both P90 and P120 in SOD1^{G93A} mice (FIG. 4I). SOD1^{G93A} mice also had more NMJs with PSC sprouts and bridges, which are sprouts that PSCs use to guide motor axons to another NMJ (FIG. 4I). Given that most NMJs are fully innervated at P90 in SOD1^{G93A} mice[[41]], these findings strongly suggest that PSCs alter their morphology and relationship with the NMJ prior to ALSdriven denervation. FIG. 4I shows fBTX-labeled AChRs and NG2⁺/S100B⁺ PSCs in P120 NG2-dsRed;S100B-GFP (WT) and NG2-dsRed;S100B-GFP;SOD1^{G93A} EDL. Analysis of SOD1^{G93A} muscle shows more PSCs/NMJ, reduced NMJ coverage (arrowhead), increased bridges between NMJs (arrows), and sprouts (asterisks). * p<0.05 versus WT. Scale bar=10 μm.

We next used transmission electron microscopy (TEM) to better discern the relationship between PSCs and the NMJ in ALS. We examined NMJs in the EDL muscle of P90 SOD1^{G93A} and control mice. In control mice, the majority of PSCs completely capped the presynapse (FIG. 9A, FIG. 9B, FIG. 9C, FIG. 9D, and FIG. 9E), as has been shown previously. In contrast, PSCs and the NMJ contain several morphological abnormalities in SOD1^{G93A} mice. There were more NMJs with PSCs only partially capping the presynapse and with processes instead found intruding into the synaptic cleft in SOD1^{G93A} compared to control mice (FIG. 9A, FIG. 9B, FIG. 9C, FIG. 9D, and FIG. 9E). PSCs also appear to be in the act of engulfing segments of the presynapse (FIG. 9A, FIG. 9B, FIG. 9C, FIG. 9D, and FIG. 9E), suggesting that PSCs may be more phagocytic in ALS. These EM data showing PSC abnormalities at otherwise healthy, innervated NMJs support our light microscopy finding (FIG. 4I) that PSCs alter their morphology prior to motor neuron degeneration in an ALS mouse model. Together with published findings[[18,21]], these data indicate that PSCs transition to a reactive phenotype in the early stages of ALS progression that may contribute to NMJ degeneration. FIG. 9A, FIG. 9B, FIG. 9C, FIG. 9D, and FIG. 9E show (in some embodiments) electron micrographs of NMJs in WT and SOD1^{G93A} EDL. Arrowhead denotes incomplete PSC coverage of synaptic cleft. Asterisk denotes PSC process insertion into the synaptic cleft. Arrow denotes axon engulfment by PSC. The percent of NMJs observed with these morphological features is displayed in the table. N=13-17 NMJs. Scale bar=1 μm.

Purinergic signaling has been shown to guide the transition of PSCs between maintenance and repair modes in healthy[[25,26]] and more acutely in ALS-afflicted NMJs[[18,24]]. We sought to identify the purinergic receptors enriched in PSCs to examine their roles in ALS. For this, we examined RNA-seq datasets that our lab has generated of control PSCs and other Schwann cells (SCs) isolated using fluorescence activated cell sorting (FACS). We found P2Y1, P2X7 and P2Y12 among the most enriched purinergic receptors in PSCs. We chose to focus on P2Y12 among these three purinergic receptors for the following reasons. (1) It has been shown to mediate inflammation and the synaptic pruning activity of microglia[[28]]. (2) Nothing is known about P2Y12 function in mediating the activity of PSCs in normal and under stress conditions that includes in ALS. (3) Targeting this receptor using an antagonist that acts only in peripheral tissues, Clopidogrel/Plavix, reduces cardiovascular risks, inflammation and fibrosis[[33]]. (4) The availability of molecular, genetic and pharmacological tools makes it possible to investigate the action of this receptor specifically in PSCs and in ALS. To validate that P2Y12 is expressed in PSCs, we performed immunolabeling for this receptor.

As shown in FIG. 7C, we found P2Y12 highly enriched at NMJs of control mice. We next compared P2Y12 levels between PSCs at NMJs of SOD1^{G93A} and control mice. Our initial analysis shows that P2Y12 trends lower in PSCs of SOD1^{G93A} compared to control mice (FIG. 7B). The trend towards reduced P2Y12 protein in PSCs in SOD1^{G93A} mice correlates with a wealth of published reports showing that this receptor is reduced in microglia shifting to an activated phenotype in response to diseases and aging[[29-31]]. Thus, our initial findings and published data support that P2Y12 may play roles in the response of PSCs to damages at NMJs, including those brought about by ALS.

In this aim, we define the relationship between P2Y12 levels in PSCs and NMJ degeneration caused by ALS. We then genetically targeted P2Y12 to assess its direct role in PSC-mediated NMJ maintenance and repair in ALS using the SOD1^{G93A} mouse model of the disease. We use newly developed mouse lines to delete P2Y12 specifically from PSCs and in a temporal manner. The data obtained here inform studies to modulate the function of P2Y12 to maximize the PSC-mediated repair actions to preserve and repair NMJs with ALS. FIG. 7A shows a plot demonstrating P2ry12 transcripts are enriched in PSCs. FIG. 7B provides a plot of the analysis of P2Y12 fluorescence intensity in GFP-labeled PSCs in EDL of WT and SOD1^{G93A} S100B-GFP mice following P2Y12 IHC. Each data point represents 1 NMJ. FIG. 7C shows representative images of P2Y12 IHC WT and SOD1^{G93A} S100B-GFP EDL. Scale bar=10 μm.

Previously Disclosed/Contemplated Research Design; Previous Aim 1A: Determining the relationship between P2Y12 levels in PSCs and NMJ degeneration in ALS. We expand on our initial observations showing downregulated expression of P2Y12 in PSCs with ALS. We examine mRNA levels of P2Y12 in addition to P2Y1, P2X7 and other purinergic receptors specifically in PSCs of SOD1^{G93A} mice. To isolate PSCs, we use SOD1^{G93A} crossed with our recently developed transgenic S100B-GFP;NG2-dsRed mouse line23 (FIG. 2A) to create S100B-GFP;NG2-dsRed;SOD1^{G93A} mice. PSCs will be FACS-isolated from lower hindlimb muscles at 3 disease stages: (1) prior to symptom onset at P70, (2) early symptomatic at P90, and (3) at the tipping point from early to late symptomatic at P11042 (FIG. 7D). FIG. 7D shows a timeline of NMJ degeneration and symptom onset in SOD1^{G93A} and PFN^{G118} V ALS mouse models. Comparisons will be made between S100B-GFP;NG2-dsRed littermates with and without the SOD1^{G93A} transgene. FACS isolation will be performed with an in-house BD Melody FACS machine using methods previously published by our lab[[23]]. We typically collect ˜5,000 cells from young adult hindlimb skeletal muscles using this method. In addition to PSCs, we will also examine levels of purinergic receptors in all other single-labeled S100B-GFP+ SCs to generate additional insights. A highly sensitive RNA isolation kit designed for low cell counts, PicoPure, will be used to collect RNA. After Reverse Transcription, our target genes will be pre-amplified and then assessed by qPCR. Based on the degree of variability that we have observed in qPCR data from this method[[23]] and with the expectation of a modest effect size, we will use 8 animals per genotype, sex, and disease stage.

Next, we analyze P2Y12 expression and distribution in PSCs in multiple ALS mouse models, including SOD1^{G93A}, PFN1^{G118V}, PrP-TDP43Q331K 43 and Stathmin-2^{-/-} [[44,45]] at the pre-symptomatic, symptom onset and symptomatic stages of these disease models. To this end, we will use RNAscope, immunohistochemistry (IHC), and Western blot (WB). For RNAscope, probes against S100B and NG2 will be used to identify PSCs. For IHC, PSCs will be identified by a combination of S100B expression and localization to the NMJ, as has been shown previously[[46-49]]. NMJs will be identified by labeling of AChRs with Alexa Fluor 647 conjugated α-bungarotoxin (fBTX). For both IHC and WB, we will use a P2Y12 antibody that clearly labels PSCs in skeletal muscle (see FIG. 7C & Key Resources). In addition to P2Y12, we will analyze other purinergic receptors that display altered mRNA levels in SOD1^{G93A} PSCs in our initial FACS experiment.

For IHC and RNAscope, images will be obtained with a Zeiss 900 Airy scan confocal microscope using 20×, 0.8 numerical aperture (NA) and 63×, 1.4 NA objectives. We will use the fractionator method to randomly sample NMJs, and we will blind data during acquisition and analysis to avoid bias. Separate statistical comparisons will be made for each ALS model (i.e., SOD1^{G93A} vs WT littermates or PFN1^{G118V} vs WT littermates, etc.). For each disease model, a two-way ANOVA will be used to evaluate the effects of genotype, disease stage (age), and interactions between genotype and disease stage. Additional information on mouse lines, antibodies and reagents can be found in the Key Resources document.

Previously Disclosed Aim 1B: Determining the direct contribution of P2Y12 in PSCs to ALS-induced NMJ degeneration. Our data have shown that P2Y12 is enriched in PSCs compared to all other SCs (FIG. 7A). Importantly, we have generated initial data indicating that P2Y12 expression is altered in PSCs in SOD1^{G93A} mice (FIG. 7B, FIG. 7C) and that targeting this receptor slows denervation of NMJs in this model (FIG. 8A). FIG. 8A shows representative images of AChR (Blue), synaptotagmin-labeled axons (red), and PSCs (green) in the EDL of P115 SOD1^{G93A};S100B-GFP mice treated with Vehicle or the P2Y12 antagonist Clopidogrel. Fewer denervated NMJs and more PSC sprouts were observed in Clopidogrel treated SOD1^{G93A};S100B-GFP mice. To better understand how P2Y12 impacts PSC function in ALS, we will specifically and temporally delete P2Y12 from PSCs in SOD1^{G93A} mice. For this, we will use a mouse line expressing CreER from the Kir4.1 promoter[[40]](Kir4.1-CreER).

This line has been crossed with a Rosa-stp-tdTomato (Ai14) reporter line. Ai14 has the dual purposes of confirming CreER activity and visualizing PSCs. Under the Kir4.1, Cre expression is highly specific to PSCs in skeletal muscles following tamoxifen treatment (FIG. 3E, FIG. 3F) [[40]]. We will cross the Ki4.1-CreER;Ai14 line with a conditional P2Y12 line (P2Y12^{fl/fl}) [[50]] to delete P2Y12 from PSCs. A second cross will be made with SOD1^{G93A} mice (FIG. 10E, Table 1). We will excise P2Y12 from PSCs at P30 (prior to NMJ degeneration) and P90 (during the early stages of NMJ denervation—see FIG. 7D) by activating Cre-ER with tamoxifen. These timepoints will allow us to evaluate P2Y12 in PSC maintenance of NMJs (prior to NMJ degeneration) or repair (during NMJ denervation). Mice will be sacrificed at P120 for evaluation of NMJ innervation, PSC morphology, and motor neuron health. FIG. 10E shows Table 1 illustrating Aim 1B Experimental Design and plan that we will cross the Ki4.1-CreER;Ai14 line with a conditional P2Y12 line (P2Y12^{fl/fl}) 50 to delete P2Y12 from PSCs. A second cross will be made with SOD1^{G93A} mice.

We will examine NMJs in both the EDL and diaphragm muscles using IHC. The progression of ALS pathology is delayed in the diaphragm relative to the EDL muscle in SOD1^{G93A} mice as the disease follows a caudal to rostral progression in these mice. These differences in ALS pathology, as well as functional demands and muscle fiber type composition, will allow us to glean as much information as possible from each mouse. The presynapse will be revealed by antibodies against synaptotagmin-2 and synaptophysin. AChRs in the postsynapse will be visualized using fBTX. PSCs will be identified by tdTom (see example in FIG. 3F). To determine the effect of deleting P2Y12 in PSCs on NMJ health, we will examine the presynapse for presence of large blebs, which are present in degenerating axons[[51]], as well as the extent of postsynaptic innervation (full, partial, absent). We will examine the ability of PSCs to maintain and repair the NMJ[[14,16]], including PSC number, the extent to which they cover the NMJ and the number of sprouting bridges they form. These methods of NMJ analysis have been used in published studies from our lab[[23,41,46,52-56]].

Transmission Electron Microscopy (TEM) has been used to determine whether P2Y12 deletion impacts PSCs on a subcellular level in the EDL. We evaluated PSC coverage of the motor axon terminal and synaptic cleft and process insertion into the synaptic cleft (see FIG. 9A, FIG. 9B, FIG. 9C, FIG. 9D, and FIG. 9E). At each NMJ, we will compare changes in PSCs with morphological signs of synaptic deterioration, including loss of innervation, disorganized or shallow postsynaptic gutters, decreased number of active zones, presence of synaptic vesicles outside of the axon terminal, and damaged mitochondria.

The health of spinal motor neurons will also be evaluated. We will count the number of motor neurons in 30 μm coronal sections collected from lumbar segments 1 to 3. We will use antibodies against NeuN and vesicular acetylcholine transferase to identify motor neurons. This method has been used in published studies by our lab[[57,58]]. We will also use anti-cleaved caspase 3 to assess the number of dying motor neurons.

Samples will be blinded, imaged and randomly sampled according to the methods described in Aim 1A. Female and male mice will be used. We will use 10 mice per genotype, age and sex for these experiments. Statistical comparisons will be made between tamoxifen-treated Kir4.1-CreER mice with and without P2Y12^{fl/fl} and vehicle treated Kir4.1-CreER; P2Y12^{fl/fl} mice (FIG. 10F, Table 2). A two-way ANOVA will be used to evaluate the effects of genotype, age of treatment, and interactions between genotype and treatment age. FIG. 10F shows Table 2 illustrating we will use 10 mice per genotype, age and sex for these experiments. Statistical comparisons will be made between tamoxifen-treated Kir4.1-CreER mice with and without P2Y12^{fl/fl} and vehicle treated Kir4.1-CreER; P2Y12^{fl/fl} mice1.

Previously Disclosed Aim 1C: Comparing motor function and the lifespan of SOD1^{G93A} mice with and without P2Y12. We will examine the effect of deleting P2Y12 in PSCs on motor function and lifespan of SOD1^{G93A} mice. We will administer Tamoxifen at p30 and evaluate body weight and motor behavior on a weekly basis starting at P70. Assays of motor function will include DigiGait to collect information on paw position and angle, stride length, speed, fore- and hind paw base width, and paw overlap, as previously described in motor function evaluations of rodent ALS models[[59]]. In addition, we will perform hindlimb clasping reflex, rotarod, and wire hang tests [[60,61]]. We will stop testing mice for motor function once they are unable to perform adequately on a rotarod and lose significant weight. For survival analysis, the lifespan of mice will be determined by their humane endpoint, which is defined by functional paralysis of both hindlimbs and loss of righting reflex within 20 seconds of being placed in lateral recumbency.

We will examine 20 mice per genotype and sex for motor function and survival analysis. The experimenter will be blinded to genotype. Statistical comparisons will be made between tamoxifen-treated Kir4.1-CreER mice with and without P2Y12^{fl/fl} and vehicle treated Kir4.1-CreER;P2Y12^{fl/fl} mice (Table 2, p30 treatment age only, FIG. 10F). Distribution normality of residuals will be assessed by Shapiro-Wilk test and QQ plots. For normally distributed data ordinary one-way ANOVA with Bonferroni post hoc will be used to evaluate the effect of genotype. For data that are not normally distributed, a Kruskal-Wallis test will be used.

Expected outcomes: We expect that our molecular analysis will show a close relationship between levels of P2Y12 and progression of NMJ degeneration in ALS. We make this prediction based on immunostaining analysis of P2Y12 indicating decreased levels in PSCs in symptomatic SOD1^{G93A} mice (see FIG. 7B, FIG. 7C) and extensive published data showing decreased levels of P2Y12 in microglia responding to stressors[[28-31]]. During the course of this study, we will also examine expression of other purinergic receptors from FACS-isolated PSCs and all other SCs from control and ALS models. If we discover that expression of other purinergic receptors is impacted by ALS pathology, we will develop strategies to modulate their activities to treat ALS in future studies. In future studies, we will seek collaborations to ascertain levels and distribution of purinergic receptors in PSCs from humans. Given the importance of purinergic signaling for PSCs to decode functional and structural changes at NMJs in healthy and ALS-affected NMJs[[18,24-26,62]], this information will provide unique insights to design future studies irrespective of outcome.

We predict that deletion of P2Y12 in PSCs will allow them to better respond to damages at NMJs caused by ALS. However, it is possible that deletion of P2Y12 will instead accelerate NMJ degeneration. This is an unlikely outcome because our initial analysis using Clopidogrel, a P2Y12 antagonist, shows preservation of NMJs in SOD1^{G93A} mice (FIG. 8A). If we find that deleting P2Y12 either slows or accelerates NMJ degeneration in ALS models, we will measure calcium-mediated activity in PSCs lacking P2Y12 in WT and SOD1^{G93A} mice. This is based on our initial findings that calcium responses in PSCs are modulated by purinergic signaling. We will also use electrophysiology to evaluate synaptic transmission in NMJs of SOD1^{G93A} mice in which P2Y12 is deleted in PSCs. We will perform these experiments in collaboration with Dr. Thomas Gould (Co-I in this application).

We expect variable penetrance of P2Y12 deletion in PSCs using the Kir1.4-CreER model. Because of this, we expect to observe high phenotypic variability following conditional P2Y12 excision. It is also possible that tamoxifen, which has been found to have a modestly positive effect on ALS, may mask the consequences of deleting P2Y12 from PSCs. If high phenotypic variability is observed, or if tamoxifen controls (see Table 2) show significant differences in PSC morphology or NMJ health versus P2Y12^{WT/WT} controls, we will employ a conditional deletion strategy that does not require Tamoxifen. For this, we will cross conditional P2Y12^{fl/fl} mice with a Sox10-Cre;Ai14 mouse line which we currently maintain in our animal colony. The Sox10 promoter will drive Cre expression in all SCs[[63]], resulting in P2Y12 deletion in both PSCs and other SCs[[50,64]]. The resulting line will be crossed with SOD1^{G93A} mice. Because P2Y12 will be deleted from all SCs during embryogenesis stages, it may affect the normal development of PSCs and NMJs. However, we believe that the combined genetic approaches along with pharmacologically targeting P2Y12 in Aim 2, will provide the necessary data to make meaningful inferences about the role of this receptor in mediating the actions of PSCs at NMJs with ALS. Our lab routinely performs the molecular, histological and behavioral analyses described here[[41,53,55-57,65]]. All mouse lines proposed in this experiment are maintained as colonies in our lab. We are therefore confident that we will complete this work regardless of outcome.

Previously Disclosed Aim 2: Testing the efficacy of the P2Y12 antagonist Clopidogrel in slowing and reversing ALS pathogenesis. OVERVIEW: We performed a pilot study to test the impact of modulating P2Y12 in PSCs with the antagonist Clopidogrel on NMJ degeneration in SOD1^{G93A} mice. Although the number of mice examined was insufficient for statistical comparison, we found that 30 days of daily Clopidogrel treatment (1 mg/kg), starting at symptom onset, reduces NMJ denervation during the symptomatic stage of this ALS mouse model (FIG. 8A). Clopidogrel treatment also increased levels of PSC sprouting, which has been shown to be a mechanism by which PSCs guide motor axons to denervated NMJs[[47,66-71]] to prolong muscle function[[72-74]]. To expand on these exciting initial findings, we will perform additional studies in Aims 2A&B to discover the optimal dose and time of clopidogrel treatment for preventing NMJ degeneration in SOD1^{G93A} mice and in another well-characterized mouse model of ALS, PFN1^{G118V}. In Aim 2C, we will examine the effect of Clopidogrel on the health and lifespan of SOD1^{G93A} and PFN1^{G118V} mice. These experiments will provide pre-clinical data using an FDA-approved drug to treat ALS.

Previously Discussed/Contemplated Research Design; Aim 2A: Determining the maximum safe Clopidogrel dose. The side effects of Clopidogrel in mice include lethargy, weight loss and internal bleeding. To determine the maximum dose with minimal side effects, we will perform a pilot study in SOD1^{G93A} and PFN1^{G118V} mice using daily I.P. administration of Clopidogrel at 1, 2 and 4 mg/kg for 20 days. This range of doses has been previously reported in mouse studies[[75]]. Mice will be monitored daily for water and food intake, bleeding, weight loss and lethargic behavior. We will use 5 SOD1^{G93A};S100BGFP and PFN1^{G118V};S100B-GFP mice per treatment and sex (male and female mice will be used).

Aim 2B: Examine the effect of Clopidogrel on NMJ health. SOD1^{G93A} and PFN1^{G118V} mice will begin daily I.P. clopidogrel treatments at either the pre-symptomatic or early symptomatic stages. This will allow us to separately evaluate the effects of Clopidogrel treatment during PSC-mediated NMJ maintenance (i.e. during the presymptomatic stage) and PSC-mediated NMJ repair (i.e. during the symptomatic stage). In SOD1^{G93A} mice, presymptomatic treatment will begin at P70 and symptomatic treatment will begin at P90. In PFN1^{G118V} mice presymptomatic and symptomatic treatments will begin at P100 and P165, respectively (see SOD1^{G93A} and PFN1^{G118V} symptom timeline in FIG. 7D). All mice will be evaluated at a fully symptomatic age in which motor deficits are evident and muscles have a mixture of innervated and denervated NMJs. This stage corresponds to P110 in SOD1^{G93A} mice and P200 in PFN1^{G118V} mice[[34,42]]. This will allow us to determine the impact of Clopidogrel on (1) preventing NMJ denervation and (2) promoting reinnervation of denervated muscles via PSC sprouting[[47,66-71]].

SOD1^{G93A} and PFN1^{G118V} lines will be crossed with the S100B-GFP line. This line will be used because GFP expression in PSCs is robust, stable and labels PSC processes far more reliably than commercially available S100B antibodies76. PSCs will be identified by transgenic GFP labeling and proximity to the NMJ, as described previously[[46,76]]. We will use the morphological analyses described in Aim 1B to determine the impact of clopidogrel on NMJ innervation, PSC coverage of the NMJ, PSC sprouting, PSC guidance of motor axons to damaged NMJs and motor neuron number in EDL and diaphragm muscles. For subcellular analysis, we will perform TEM on the EDL, as described in Aim 1B.

Assignment of Clopidogrel and vehicle treatments will be randomized. Samples will be blinded, imaged and randomly sampled according to the methods described in Aim 1A. Female and male mice will be used. We will use 10 SOD1^{G93A};S100B-GFP and PFN1^{G118V};S100B-GFP mice per treatment, treatment time point, and sex. SOD1^{G93A} and PFN1^{G118V} mice will be analyzed separately using two-way ANOVA to evaluate the effects of treatment, age of treatment, and interactions between treatment and age of treatment.

Previously Disclosed Aim 2C: Examining the effect of Clopidogrel on the health and lifespan of SOD1^{G93A} and PFN1^{G118V} mice. We will determine whether Clopidogrel treatment impacts progressive loss of motor function and lifespan in SOD1^{G93A} and PFN1^{G118V} mice. This will allow us to evaluate the extent to which any improvements in motor neuron or NMJ health found in Aim 2A translate into functional improvements. As in Aim 2B, Clopidogrel treatment will begin in either the pre-symptomatic or early symptomatic stage.

During the course of drug treatments, we will evaluate motor behavior and mouse weight on a weekly basis. We will use DigiGait, hindlimb clasping reflex, rotarod and inverted wire screen tests of motor behavior as described in Aim 1C. We will track lifespan by recording the age at which the mice reach their humane endpoint, as described in Aim 1C. After mice have been euthanized, we will weigh muscles (TA and soleus) to determine if these treatments prevent loss of muscle mass. We will examine NMJs, motor neurons and glial cells using the methods described in Aim 1B. We will also collect fresh frozen (unfixed) muscles from one leg for molecular analyses of markers associated with degenerating NMJs and muscle atrophy we and others have used in previous studies[[51,65]]. These will include expression analysis of the gamma AChR subunit and the Muscle Specific Kinase (MuSK) for NMJs[[77,78]] and Foxo1 and MuRF1 for muscle atrophy[[79,80]].

Clopidogrel and vehicle treatments will be randomized. We will treat 20 mice per genotype and sex with Clopidogrel or vehicle. Statistical comparisons will be performed according to Aim 2B.

Expected outcomes: Based on our discovery that P2Y12 is highly expressed by PSCs (FIG. 7D) and our data showing that Clopidogrel treatment reduces NMJ denervation in symptomatic SOD1^{G93A} mice (FIG. 8A), we expect that modulating P2Y12 activity with Clopidogrel will delay motor neuron death and muscle fiber denervation and extend the lifespan of SOD1^{G93A} and PFN1^{G118V} mice. However, it is possible that decreased P2Y12 expression PSCs is a deleterious reaction to disease pathology that inhibits their ability to repair NMJs. In that case, we will examine whether a P2Y12 agonist, rather than an antagonist, may have a positive effect on NMJs in ALS. Regardless of outcome, these experiments will demonstrate the impact of modulating PSC-enriched purinergic receptors on NMJs in ALS. They will also provide valuable information for when to start treatments to maximize the positive effects of a given drug.

Clopidogrel does not cross the blood-brain-barrier (BBB), however increased BBB leakiness during disease progression may cause it to have direct effects on glia of the spinal cord. To address this possibility, we will examine spinal cord microglia using Iba1, Cd11 b and P2Y12 antibodies, a combination of markers that reveal homeostatic and hyperactive microglia[[81,82]]. Given that the Clopidogrel is administered systemically, we cannot rule out that it impacts PSCs and the NMJ indirectly by altering fibroblasts in skeletal muscles, the vasculature, or other peripheral organs[[33]]. In that case, even if it were administered intramuscularly it would be difficult to determine whether PSCs are primarily responsible for the negative or positive effects of Clopidogrel. If a treatment effect is observed, we will perform ex vivo experiments to directly test the impact of Clopidogrel on PSC calcium responses to neurotransmission. We will also examine the effect of Clopidogrel on the morphology of PSCs and their secretion of inflammatory molecules. In vivo, we will use electrophysiology to evaluate synaptic transmission in NMJs and ensuing response of PSCs exposed to Clopidogrel. We will perform these experiments in collaboration with Dr. Thomas Gould (Co-I in this application). Additionally, we will examine the effect of Clopidogrel on NMJs and motor neurons in addition to the health and lifespan of other ALS mouse models, such as PrP-TDP43^{Q331K} [[43]] and Stathmin-2^{-/-}[[44,45]] mice. Finally, we will design future studies to determine whether Clopidogrel used together with other drugs that target mechanisms in the central nervous system, such as Riluzole, provide even more protection to NMJs and motor neurons dealing with ALS-causing factors.

Previously Disclosed Aim 3: Identifying downstream effectors of P2Y12 in PSCs dysregulated in ALS. OVERVIEW: Purinergic signaling has been shown to modulate intracellular calcium responses to neurotransmission as well as expression of cytokines and growth factors in PSCs and other glia[[62]]. It also regulates other signaling pathways, such as PI3K, that in turn affect levels of purinergic receptors[[83]]. Despite these advances, little is known about the molecular pathways P2Y12 recruits to influence the function of PSCs. This has been due to the inability to isolate PSCs from other glia of the PNS. Discovering these signaling pathways is not only essential for our basic understanding of PSC biology, it is crucial for developing therapeutic approaches that target purinergic signaling in PSCs in ALS. In this aim, we will use our newly developed method of isolating PSCs[[23]] in combination with single-cell RNA-seq to identify molecular pathways that link purinergic signaling to disease-associated changes in PSC function.

Previously Contemplated/Disclosed Research Design for Aim 3A: Performing scRNA-seq and scATAC-seq. Single-cell RNA-seq (scRNA-seq) and Assay for Transposase Accessible Chromatin (scATAC-seq) will be performed on FACS-isolated PSCs and SCs from control and SOD1^{G93A} mice. We will use the genetic and pharmaceutical approach to modulate P2Y12 activity, described in Aims 1 and 2, to interrogate pathways altered in PSCs by these interventions in the SOD1^{G93A} ALS model. Specifically, we will use the Kir4.1-Cre;P2Y12^{fl/fl} control and SOD1^{G93A} lines developed in Aim 1 and Chlopidogrel, used in Aim 2. All S100B-GFP+(Clopidogrel model) or Sox10-Cre;tdTom⁺ (Sox10-Cre;P2Y12^{fl/fl} model) PSCs and SCs will be collected. PSCs will be differentiated from other SCs at the data analysis level by NG2 expression, which is unique to PSCs. These cells will be FACS-isolated from the TA muscle at the presymptomatic (P70) and early symptomatic (P90) ages. We routinely isolate ˜5,000 PSCs and ˜10,000 of the other SCs from the TA of S100-GFP;NG2-dsRed mice. Thus, we do not foresee issues in obtaining sufficient cells to perform single-cell molecular profiling from only S100B-GFP mice.

We will simultaneously profile gene expression and chromatin accessibility using the Chromium Single Cell ATAC Multiome+Gene Expression platform from 10×Genomics. This method analyzes the transcriptome and chromatin accessibility for the same nucleus. To do so, we will extract nuclei from 10,000 cells/mouse using the Nuclei PURE Isolation Kit (Sigma-Aldrich) and perform transposition to selectively target open chromatin regions (ATAC library) and barcode mRNA (gene expression library). Sequencing depths will be 20,000 read pairs/cell for gene expression and 25,000 read pairs/cell for ATAC sequencing. Programs for data analysis will include Cell Ranger ARC program (10×Genomics) for chromatin accessibility and gene expression profiling, Loupe Browser (10×Genomics) for PSC subgrouping, and both DeepChrome84 and TIMEOR85 to identify gene regulatory networks that are altered by purinergic receptor modulation. We will use an adjusted P value of <0.5 and the log 2 fold change of >0.5 or <0.5 to consider genes expression significantly different between genotypes, treatments and sex. We will use other tools, such as EnrichR v.30 for functional enrichment analysis, as needed and based on advice from Dr. Ashley Webb (see letter of support) and the bioinformatic core at Brown University, to analyze, group and interpret datasets. We will examine at least 4 mice per genotype, treatment, age and sex.

Previously Disclosed Aim 3B: Identifying and validating transcriptional targets of purinergic receptors in ALS-affected PSCs. To identify downstream effectors of P2Y12, candidate genes must meet all 3 of the following criteria: 1) Expression is altered by modulating this receptor in non-SOD1^{G93A} controls; 2) Expression is altered by the SOD1^{G93A} transgene; and 3) Modulation of the purinergic receptor prevents the change in expression induced by the SOD1^{G93A} transgene. We will further narrow the list of genes using the same criteria for chromatin modifications. Genes with known or predicted functions in synaptic maintenance and repair will be prioritized. These will include genes encoding cytoskeletal molecules, scaffolding proteins, secreted factors, and receptors that we have previously identified in PSCs from control mice[[23]]. In addition to genes and pathways regulated by purinergic signaling, this approach will reveal other genes and signaling pathways dysregulated in PSCs of SOD1^{G93A} mice that may synergize with purinergic signaling to affect the function of PSCs in ALS.

We will use qPCR to validate expression changes of candidate genes in FACS-isolated PSCs according to methods previously published by our lab[[23]]. In addition, RNAscope and IHC will be used whenever possible to link changes in gene expression with ALS-related morphological changes in PSCs and NMJ degeneration. Validation experiments will be performed in the TA and diaphragm muscles of P70 and P90 SOD1^{G93A} mice. Validated genes will be further examined in PSCs from other mouse models of ALS, such as PFN1^{G118V}, PrPTDP43Q331K and Stathmin-2^{-/-} mice treated with Clopidogrel and vehicle. These experiments will reveal candidate genes P2Y12 uses to mediate the actions of PSCs in ALS. In future studies, we will examine the function of such candidate genes in affecting the onset and progression of NMJ degeneration in ALS.

Expected outcomes: Our single cell approach utilizes the S100B (Clopidogrel model) or Sox10 (Kir4.1 CreER model) promoters to collect both PSCs and other SCs into a single, heterogenous pool for analysis. Despite the fact that we are analyzing a heterogenous pool of PSCs and other SCs, we do not expect any difficulty in collecting sufficient numbers of PSCs for analysis or differentiating PSCs from other SCs. This is because we typically observe a 1:2 ratio of PSCs to other SCs when FACS isolating these cells from S100B-GP;NG2-dsRed mice (in which PSCs are distinguished from other SCs by dsRed). This ratio will provide ˜5,000 PSCs, which exceeds 10×Genomics recommendations for SC-RNA-seq. We will differentiate PSCs from other SCs by NG2 expression, which continues to be expressed in PSCs symptomatic SOD1^{G93A} mice (FIG. 11G, FIG. 11H). We expect to identify unique molecular pathways P2Y12 uses to mediate the actions of PSCs at NMJs in ALS. Among these, we expect to uncover changes in signaling molecules, including secreted molecules and receptors, with critical roles in maintaining PSC and NMJs. However, it is worth noting that scRNA-seq and scATAC-seq analyses pose two primary concerns. First, these techniques lack adequate read depth to capture lowly expressed transcripts[[86]]. Second, our combined scRNA-seq and scATAC-seq approach works only on nuclei and the exclusion of cytosolic transcripts further dilutes the coverage of the transcriptome. We deem these as minor concerns given that our chromatin modification analysis (scATAC) will provide additional leads about genes and molecular pathways regulated by purinergic signaling in PSCs. If the Tamoxifen-treated Kir4.1-CreER model is shown to be unsuitable in Aim 1B we will use Sox10-Cre mice instead. Altogether, the data collected in this aim will serve as the foundation for future studies to target purinergic signaling effectors in PSCs to preserve and repair NMJs.

This project provides basic and translational data about the role of purinergic signaling in PSCmediated NMJ maintenance and repair in ALS. It will also be the first to define the molecular changes that precipitate PSC dysfunction in ALS. In each Specific Aim, we have included alternative approaches and future directions. We will perform these studies in both female and male mice. We will use the necessary number of mice to power each experiment. For most experiments, we will use the appropriate ANOVA along with Bonferroni post-hoc analysis to assess for potential statistical differences between groups. The number of mice and specific statistical analyses are detailed in each Aim and in the Vertebrate Animal Document. Additionally, we will provide qualitative information when necessary regarding the morphology of PSCs, NMJs and motor neurons in the ALS mouse models with and without altered activity of PSC-enriched P2Y12. We will blind experimenters during data collection and analysis. Altogether, the studies and rigor in this proposal will generate significant basic and translational knowledge to slow, stop and reverse the ravages of ALS on PSCs, NMJs and skeletal muscles. Our efforts are going to be aided by Dr. Gould (Co-I), expert on purinergic signaling in PSCs, Dr. Webb (collaborator), expert on single-cell molecular analysis, and the ALS Therapy Development Institute working closely with Dr. Vieira (CEO and CSO) and Dr. Hatzipetros (Director of Pharmacology).

While providing a Detailed Description, in a discussion, study or a reading of the details, features, embodiments, aspects, and/or examples of the technology disclosed herein, any of the features, embodiments, aspects, and/or examples can be inter-combined (or inter-discussed) with the example details listed below:
Detail 1: A method of treating a neuromuscular condition or a nervous condition in a subject, comprising: administering to the subject a precisely calculated therapeutically effective amount of at least one of Clopidogrel, Ticagrelor, Prasugrel, Cangrelor, or a pharmaceutically acceptable salt, prodrug, hydrate, or solvate thereof, wherein the therapeutically effective amount is determined based on the subject's age, weight, gender, severity of the condition, and overall health status, wherein the at least one of Clopidogrel, Ticagrelor, Prasugrel, Cangrelor, or a pharmaceutically acceptable salt, prodrug, hydrate, or solvate thereof selectively targets and binds to specific receptors in the neuromuscular junctions, thereby preserving and/or repairing the structural integrity and functional capacity of the neuromuscular junctions in the subject, and wherein the administration is performed using a carefully controlled delivery system that ensures the therapeutically effective amount reaches the targeted neuromuscular junctions at a steady and consistent rate over a prolonged period of time.
Detail 2: The method of detail 1, wherein the subject is definitively diagnosed with the neuromuscular condition or the nervous condition using a combination of advanced diagnostic techniques, including genetic testing, electromyography, nerve conduction studies, and muscle biopsy, to ensure accurate identification of the specific condition affecting the subject.
Detail 3: The method of detail 1, wherein the subject is suspected of having the neuromuscular condition or the nervous condition based on a comprehensive analysis of the subject's medical history, family history, lifestyle factors, environmental exposures, and presenting symptoms, using a sophisticated artificial intelligence algorithm that compares the subject's data to a vast database of known cases to determine the likelihood of the condition's presence.
Detail 4: The method of detail 1, wherein the subject has a significantly elevated likelihood of developing the neuromuscular condition or the nervous condition due to the presence of specific genetic mutations, biomarkers, or risk factors that have been identified through extensive epidemiological studies and validated by multiple independent research groups using large, diverse patient cohorts and rigorous statistical analysis.
Detail 5: The method of detail 1, wherein administering the therapeutically effective amount reduces a number of denervated neuromuscular junctions in the subject by a clinically significant percentage, as determined by comparing the number of denervated junctions before and after treatment using high-resolution imaging techniques such as electron microscopy and immunohistochemistry, with the results being statistically analyzed to ensure the reduction is not due to chance or natural variation.
Detail 6: The method of detail 1, wherein the at least one of Clopidogrel, Ticagrelor, Prasugrel, Cangrelor, or a pharmaceutically acceptable salt, prodrug, hydrate, or solvate thereof comprises Clopidogrel or a pharmaceutically acceptable salt, prodrug, hydrate, or solvate thereof, and wherein the Clopidogrel is in the form of a highly purified, stereochemically pure S-enantiomer that has been optimized for maximum potency and minimum side effects through extensive structure-activity relationship studies and advanced formulation techniques.
Detail 7: The method of detail 1, wherein the at least one of Clopidogrel, Ticagrelor, Prasugrel, Cangrelor, or a pharmaceutically acceptable salt, prodrug, hydrate, or solvate thereof comprises Ticagrelor or a pharmaceutically acceptable salt, prodrug, hydrate, or solvate thereof, and wherein the Ticagrelor is chemically modified with a targeted delivery moiety that specifically binds to receptors expressed on the surface of neuromuscular junctions, allowing for enhanced accumulation and retention of the drug at the site of action.
Detail 8: The method of detail 1, wherein the at least one of Clopidogrel, Ticagrelor, Prasugrel, Cangrelor, or a pharmaceutically acceptable salt, prodrug, hydrate, or solvate thereof comprises Prasugrel or a pharmaceutically acceptable salt, prodrug, hydrate, or solvate thereof, and wherein the Prasugrel is co-administered with a synergistic agent that potentiates its effects on neuromuscular junction preservation and repair, with the optimal ratio of Prasugrel to the synergistic agent being determined through extensive dose-response studies and pharmacokinetic/pharmacodynamic modeling.
Detail 9: The method of detail 1, wherein the at least one of Clopidogrel, Ticagrelor, Prasugrel, Cangrelor, or a pharmaceutically acceptable salt, prodrug, hydrate, or solvate thereof comprises Cangrelor or a pharmaceutically acceptable salt, prodrug, hydrate, or solvate thereof, and wherein the Cangrelor is formulated in a long-acting, sustained-release dosage form that maintains therapeutic concentrations of the drug at the neuromuscular junctions for an extended duration, as determined by in vivo imaging and pharmacokinetic studies.
Detail 10: The method of detail 1, wherein the neuromuscular condition is selected from the group consisting of amyotrophic lateral sclerosis, spinal muscular atrophy, and Duchenne muscular dystrophy, and wherein the specific condition is definitively diagnosed using a comprehensive battery of tests that includes genetic sequencing, electrophysiological studies, imaging analyses, and histopathological examinations of muscle and nerve tissue biopsies.
Detail 11: The method of detail 1, wherein the nervous condition is selected from the group consisting of Alzheimer's disease, Parkinson's disease, and Huntington's disease, and wherein the specific condition is definitively diagnosed using a combination of clinical assessments, neuropsychological tests, brain imaging studies, and biomarker analyses that are interpreted by a multidisciplinary team of experts using standardized diagnostic criteria.
Detail 12: The method of detail 1, wherein the therapeutically effective amount is administered orally, parenterally, subcutaneously, intravenously, intramuscularly, intraperitoneally, transdermally, intranasally, or rectally, using a dosage form and route of administration that are selected based on the subject's individual characteristics and preferences, as well as the pharmacokinetic and pharmacodynamic properties of the specific drug being administered, with the goal of maximizing therapeutic efficacy and minimizing adverse effects.
Detail 13: The method of detail 1, wherein the therapeutically effective amount is administered in a dosage form selected from the group consisting of a tablet, a capsule, a solution, a suspension, an emulsion, a powder, a granule, a suppository, an injection, an inhalant, and a transdermal patch, and wherein the dosage form is engineered using advanced manufacturing techniques such as 3D printing, nanotechnology, and microfluidics to optimize its release kinetics, stability, and bioavailability.
Detail 14: A method of reducing denervation of neuromuscular junctions in a subject, comprising: administering to the subject a rigorously determined therapeutically effective amount of at least one of Clopidogrel, Ticagrelor, Prasugrel, Cangrelor, or a pharmaceutically acceptable salt, prodrug, hydrate, or solvate thereof, wherein the therapeutically effective amount is established through a series of carefully controlled clinical trials that systematically evaluate the safety and efficacy of different doses and dosing regimens in large, diverse patient populations, wherein the at least one of Clopidogrel, Ticagrelor, Prasugrel, Cangrelor, or a pharmaceutically acceptable salt, prodrug, hydrate, or solvate thereof reduces a number of denervated neuromuscular junctions in the subject by a statistically significant and clinically meaningful margin, as assessed by multiple independent and blinded evaluators using validated, quantitative outcome measures.
Detail 15: The method of detail 14, wherein the subject is diagnosed with a neuromuscular condition or a nervous condition based on a definitive and unambiguous set of diagnostic criteria that have been established by international consensus panels of leading experts in the field and validated through extensive clinical testing and real-world experience.
Detail 16: The method of detail 14, wherein the subject is suspected of having a neuromuscular condition or a nervous condition based on a rigorous differential diagnosis that systematically rules out other potential causes of the subject's symptoms and signs, using a comprehensive battery of diagnostic tests and a standardized, evidence-based decision-making algorithm.
Detail 17: The method of detail 14, wherein the subject has a likelihood of developing a neuromuscular condition or a nervous condition that exceeds a precisely defined threshold level, as determined by a sophisticated risk prediction model that incorporates a wide range of genetic, epigenetic, demographic, environmental, and lifestyle factors and has been extensively validated in multiple independent patient cohorts.
Detail 18: The method of detail 14, wherein administering the therapeutically effective amount preserves and/or repairs neuromuscular junctions in the subject, as demonstrated by a significant improvement in one or more clinically relevant parameters such as muscle strength, endurance, and electrophysiological function, using standardized, validated assessment tools and protocols.
Detail 19: The method of detail 14, wherein the neuromuscular condition is selected from the group consisting of amyotrophic lateral sclerosis, spinal muscular atrophy, and Duchenne muscular dystrophy, and wherein the specific condition is classified into a precise subtype and stage based on a comprehensive set of molecular, physiological, and clinical criteria that have been established by international expert consensus and validated through extensive research.
Detail 20: The method of detail 14, wherein the nervous condition is selected from the group consisting of Alzheimer's disease, Parkinson's disease, and Huntington's disease, and wherein the specific condition is definitively diagnosed using a multimodal approach that integrates clinical, imaging, biochemical, and histopathological data, with the diagnosis being confirmed by an independent panel of experts using standardized, validated diagnostic algorithms.
Detail 21: The method of detail 1, wherein the therapeutically effective amount is determined using a personalized dosing regimen that is based on the subject's unique genetic profile, metabolic characteristics, and disease-specific factors, as determined by advanced pharmacogenomic and pharmacometabolomic testing and sophisticated computational modeling techniques.
Detail 22: The method of detail 1, wherein the at least one of Clopidogrel, Ticagrelor, Prasugrel, Cangrelor, or a pharmaceutically acceptable salt, prodrug, hydrate, or solvate thereof is administered in combination with one or more additional therapeutic agents that act synergistically to enhance the preservation and repair of neuromuscular junctions, with the specific combination and dosing regimen being optimized through extensive preclinical and clinical testing.
Detail 23: The method of detail 1, wherein the administration is performed using an advanced drug delivery system that enables precise spatial and temporal control of drug release, such as a targeted nanoparticle formulation or a programmable microchip device, based on real-time feedback from biosensors that continuously monitor the subject's physiological status and therapeutic response.
Detail 24: The method of detail 1, wherein the subject's response to the treatment is continuously monitored using a comprehensive set of biomarkers and clinical outcome measures, with the data being analyzed in real-time using machine learning algorithms to optimize the dosing regimen and maximize therapeutic efficacy while minimizing adverse effects.
Detail 25: The method of detail 1, wherein the treatment is administered as part of a comprehensive, multidisciplinary care plan that includes dietary modifications, physical therapy, occupational therapy, speech therapy, and psychosocial support, with all aspects of the plan being carefully coordinated and customized to the subject's individual needs and goals.
Detail 26: The method of detail 14, wherein the number of denervated neuromuscular junctions is quantified using a combination of high-resolution imaging techniques, electrophysiological assessments, and histomorphometric analyses, with the data being subjected to rigorous statistical analysis to ensure the reliability and validity of the results.
Detail 27: The method of detail 14, wherein the therapeutically effective amount is administered using a pulsatile dosing regimen that is designed to minimize the development of drug tolerance and maximize the long-term efficacy of the treatment, based on a thorough understanding of the pharmacokinetic and pharmacodynamic properties of the drug and the pathophysiological mechanisms underlying the disease.
Detail 28: The method of detail 14, wherein the at least one of Clopidogrel, Ticagrelor, Prasugrel, Cangrelor, or a pharmaceutically acceptable salt, prodrug, hydrate, or solvate thereof is administered in a formulation that is specifically designed to enhance its uptake and retention at the neuromuscular junctions, such as a liposomal or nanoparticulate formulation that is conjugated with targeting ligands that bind to specific receptors on the junctional membrane.
Detail 29: The method of detail 14, wherein the subject's response to the treatment is evaluated using a battery of functional assessments that are specifically designed to measure the strength, endurance, and coordination of the affected muscles, as well as the subject's overall quality of life and ability to perform activities of daily living.
Detail 30: The method of detail 14, wherein the treatment is administered in a carefully controlled clinical setting, with the subject being closely monitored for any signs of adverse effects or complications, and with the dosing regimen being adjusted as necessary based on the subject's individual response and tolerability.
Detail 31: A method of treating a neuromuscular condition or a nervous condition in a subject in need thereof, the method comprising: administering to the subject a precisely calculated therapeutically effective amount of a highly specific P2Y12 inhibitor selected from the group consisting of the potent antiplatelet agents Clopidogrel, Ticagrelor, Prasugrel, Cangrelor, and their pharmaceutically acceptable salts, biologically active prodrugs, stable crystalline hydrates and solvates thereof, wherein the P2Y12 inhibitor is administered orally, parenterally, intravenously, intramuscularly, transdermally or via inhalation at a carefully controlled dosage ranging from 0.1 mg to 1000 mg per day in single or divided doses, whereby, through the precise inhibition of the P2Y12 receptor and the consequent modulation of purinergic signaling pathways, the structural integrity and functional viability of neuromuscular junctions are effectively preserved and/or extensively repaired or a significant number of pathologically denervated neuromuscular junctions are markedly reduced in the subject, thereby alleviating the debilitating symptoms and halting the progressive deterioration associated with the neuromuscular condition or the nervous condition.
Detail 32: The method of detail 31, wherein the subject is definitively diagnosed with the specific neuromuscular condition or the particular nervous condition using a comprehensive battery of diagnostic tests including detailed clinical examination, electrophysiological studies, imaging techniques, biochemical assays and genetic analyses.
Detail 33: The method of detail 31, wherein the subject is strongly suspected of having the neuromuscular condition or the nervous condition based on the presence of characteristic signs and symptoms, suggestive findings on preliminary investigations and a high index of clinical suspicion.
Detail 34: The method of detail 31, wherein the subject has a significantly elevated likelihood of developing the neuromuscular condition or the nervous condition due to a positive family history, the presence of established risk factors, or the detection of specific biomarkers or genetic susceptibility variants.
Detail 35: The method of detail 31, wherein the P2Y12 inhibitor is the thienopyridine derivative Clopidogrel, or a pharmaceutically acceptable salt, biologically active prodrug, stable crystalline hydrate or solvate thereof, administered at a daily dosage equivalent to 75-300 mg of the base form.
Detail 36: The method of detail 31, wherein the P2Y12 inhibitor is the cyclopentyl-triazolo-pyrimidine Ticagrelor, or a pharmaceutically acceptable salt, biologically active prodrug, stable crystalline hydrate or solvate thereof, administered at a daily dosage equivalent to 60-180 mg of the base form.
Detail 37: The method of detail 31, wherein the P2Y12 inhibitor is the thienopyridine derivative Prasugrel, or a pharmaceutically acceptable salt, biologically active prodrug, stable crystalline hydrate or solvate thereof, administered at a daily dosage equivalent to 5-10 mg of the base form.
Detail 38: The method of detail 31, wherein the P2Y12 inhibitor is the ATP analogue Cangrelor, or a pharmaceutically acceptable salt, biologically active prodrug, stable crystalline hydrate or solvate thereof, administered intravenously at an initial bolus dose of 30 mcg/kg followed by a continuous infusion of 4 mcg/kg/min for the duration of therapy.
Detail 39: The method of detail 31, wherein the neuromuscular condition is selected from the group consisting of the relentlessly progressive and invariably fatal amyotrophic lateral sclerosis, the most common inherited cause of infant mortality spinal muscular atrophy, the severely disabling and life-limiting Duchenne muscular dystrophy, the milder variant Becker muscular dystrophy, the autoimmune disorder myasthenia gravis, the hereditary neuropathy Charcot-Marie-Tooth disease, and the X-linked recessive disorder spinal and bulbar muscular atrophy.
Detail 40: The method of detail 31, wherein the nervous condition is selected from the group consisting of the chronic neurodegenerative disorder Parkinson's disease characterized by tremor, rigidity and bradykinesia, the most common cause of dementia Alzheimer's disease marked by progressive cognitive decline and memory loss, the inherited neurodegenerative disorder Huntington's disease associated with involuntary choreiform movements and behavioral changes, and the immune-mediated demyelinating disorder multiple sclerosis leading to a wide range of neurological deficits.
Detail 41: The method of detail 31, wherein the therapeutically effective amount of the P2Y12 inhibitor is administered in combination with one or more additional therapeutic agents selected from the group consisting of neuroprotective agents, anti-inflammatory drugs, immunomodulators, muscle relaxants, cholinesterase inhibitors, dopaminergic agonists, anticholinergics, tetrabenazine, baclofen, riluzole, edaravone, nusinersen, eteplirsen, golodirsen, intravenous immunoglobulin, plasmapheresis, corticosteroids, azathioprine, mycophenolate mofetil, rituximab, interferon beta, glatiramer acetate, mitoxantrone, and physiotherapy.
Detail 42: The method of detail 31, further comprising monitoring the clinical response of the subject to the administered P2Y12 inhibitor using validated assessment scales, electrophysiological studies and imaging techniques to objectively document stabilization or improvement in neuromuscular function, muscular strength, respiratory capacity, ambulation, dexterity, cognition, behavior and quality of life.
Detail 43: The method of detail 31, further comprising determining the optimal maintenance dose of the P2Y12 inhibitor for long-term therapy by carefully titrating the dosage based on the individual subject's clinical response, tolerability profile, pharmacogenetic factors influencing drug metabolism, and any potential drug-drug interactions.
Detail 44: The method of detail 31, wherein the P2Y12 inhibitor is administered as a component of a comprehensive care plan integrating pharmacological treatment with supportive measures such as physical therapy, occupational therapy, speech therapy, nutritional support, respiratory care, orthotic devices, home adaptations and psychosocial interventions to optimize functional independence and quality of life.
Detail 45: The method of detail 31, wherein the P2Y12 inhibitor is administered prophylactically to a subject with a known genetic predisposition or multiple risk factors for developing the neuromuscular condition or the nervous condition, in order to delay the onset, attenuate the severity or prevent the manifestation of clinically overt disease.

The invention provides a method for treating neuromuscular or nervous conditions by administering a therapeutically effective amount of Clopidogrel, Ticagrelor, Prasugrel, Cangrelor, or their derivatives. The dosage is tailored based on individual patient characteristics such as age, weight, and health status. The treatment targets neuromuscular junctions to preserve and repair their function, using a controlled delivery system for consistent drug release. Diagnosis of the condition is achieved through advanced techniques, and the treatment aims to reduce denervation of neuromuscular junctions significantly. The method includes various administration routes and dosage forms, optimized for efficacy and minimal side effects. The approach is part of a comprehensive care plan, potentially involving additional therapeutic agents and advanced drug delivery systems, with continuous monitoring and adjustment based on patient response.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. The methods, kits, formulations, and devices disclosed herein can be combined in any way into systems to address the current public health emergency.

The technology described herein is further illustrated by the following examples which in no way should be construed as being further limiting. The Examples are provided to demonstrate examples of future planned work, which in some experiments is emergency work. Although methods and materials similar or equivalent to those described herein can be used in the practice or testing of this disclosure, suitable methods and materials are described below.

### EXAMPLES

The invention now being generally described, it will be more readily understood by reference to the following examples which are included merely for purposes of illustration of certain aspects and embodiments of the present invention and are not intended to limit the invention.

### Example 1 Mouse Model of ALS

FIGS. 11A-11F provide a summary of Clopidogrel treatment on a mouse model of ALS. FIG. 11A (control, left) and FIG. 11B (Clopidogrel, right) provide images showing neuromuscular junctions (NMJs) treated with vehicle (control, left) and Clopidogrel (right). Blue (or grey in upper panels) depicts axons and Magenta (grey in lower panels) muscle region in contact with motor axons. FIG. 11C and FIG. 11E are plots showing muscle regions are more intact in mice treated with Clopidogrel (“Clopid” label in plots). FIG. 11D and FIG. 11F are plots showing Clopidogrel reduces the number of denervated NMJs, and hence slows a hallmark of ALS pathogenesis.

### Example 2 Prophetic Prodrug and Precursor Development

In a prophetic example, chemical derivatives and mixtures of the therapeutic agents are developed as precursors (or prodrugs). The prodrug can be converted (to active) by an enzymatic activity of the host subject, a release from a carrier, action of a cell, a digestion such as by an amylase, or can be converted by a hydrolysis, wherein a hydroxyl in the parent compound is presented as an ester or a carbonate or carboxylic acid present in the parent compound is presented as an ester. Particles (such as silica) or even a food may be used as carriers/nanocarriers of the therapeutic agent(s) for prodrug/precursor experiments. A precursor or prodrug can be metabolized to the active parent compound (therapeutic agent) in vivo (e.g., the ester is hydrolyzed to the corresponding hydroxyl, or carboxylic acid). Formulation of a precursor/prodrug is accomplished by mixing the subject agent with a carrier/nanocarrier, by mixing with an acid and/or an alcohol (then testing hydrolysis at various pH), or by attaching an enzymatically cleavable functional group. The design of prodrugs requires consideration of the following: (1) can the therapeutic agent be modified, (2) what effect will the modification have on the ADMET (absorption, distribution, metabolism, excretion, and toxicity), and (3) can the parent therapeutic agent be regenerated efficiently without producing toxic by-products. Chemical modification of the proposed agent requires a suitable functional group that allows reaction to form the targeted pro-moiety that bestows the desired ability for hydrolysis back to the therapeutic agent. Example groups include hydroxyls, amines, carboxylic acids, esters, thiols, and carbonyls. The purpose of the targeted promoiety generally dictates its targeted functions in formulation. For example, increased membrane permeability of a hydrophilic drug can be accomplished through lipidation (decreasing hydrophilicity), typically via ester bond formation. In doing so, the prodrug offers improved bioavailability through enhanced absorption from the gastrointestinal tract into systemic circulation or via topical application. Other routes to improve ADMET properties include the addition of ionizable groups to increase solubility (increasing hydrophilicity). The masking of metabolically labile groups is also investigated to prevent their premature breakdown. Future work contemplates the conjugation of peptidic epitopes for the active targeting of specific tissue/cell surface receptors and the site-sensitive activation of the precursor/prodrug for selective release.

A therapeutic agent is further examined without the ester (in a charged form) to determine various penetrations into cells and if blood brain barrier (BBB) penetration can be controlled using either de-esterification, saponification, charge, or a prodrug as described above. Drugs that target the same receptor as Clopidogrel are tested to see if these will work effectively or with other advantages. They are Ticagrelor, Prasugrel, and Cangrelor.

### Example 3 Example Statistical Analysis

GraphPad Prism version 9.5.0 can be used for statistical analyses and graphical representation (GraphPad, San Diego, CA, USA). Data can be presented as means±standard deviation (SD) or standard error of the mean (SEM). The relations between groups can be compared using two-tailed, paired Student's T tests or one-way ANOVA tests. Survival can be analyzed with the Kaplan-Meier method and compared with the log-rank test. For multiple testing, Tukey's or Benjamini-Hochberg's methods can be employed. Statistical significance can be, for example, reported as follows: p≤0.05: *, p≤0.01: **, and p≤0.001: ***.

### REFERENCES

1. Zarel, S. et al. A comprehensive review of amyotrophic lateral sclerosis. Surg. Neurol. Int. 6, 171 (2015).
2. Tumer M. R. & Swash, M. The expanding syndrome of amyotrophic lateral sclerosis a clinical and molecular odyssey. J. Neurol. Neurosurg. Psychiatry 86, 667-673 (2015).
3. Takei, K. et al. Edaravone and its clinical development for amyotrophic lateral sclerosis. Amyotrophic Lateral Sclerosis and Frontotemporal Degeneration vol. 18 5-10 (2017)
4. Montes. J. et al. Translational research in ALS. in Animal and Translational Models for CNS Drug Discovery vol. 2 267-310 (Elsevier Inc., 2008).
5. Taylor, J. P., Brown, R. H. & Cleveland, D. W. Decoding ALS: from genes to mechanism. Nature 539, 197-208 (2016).
6. Moloney, E. B., de Winter, F. & Verhaegen, J. ALS as a distal axonopathy: molecular mechanisms affecting neuromuscular junction stability in the presymptomatic stages of the disease. Front. Neurosci. 8, 252 (2014).
7. Darabid, H., Perez-Gonzalez, A. P. & Robitaille. R. Neuromuscular synaptogenesis: coordinating partners with multiple functions. Nat. Rev. Neurosci. 15, 703-18 (2014).
8. Arbour, D., Tremblay, E., Martineau, E., Julien, J.-P. & Robitaille, R. Early and persistent abnormal decoding by glial cells at the neuromuscular junction in an ALS model. J. Neurosci. 35, 688-706 (2015).
9. Park. S. at al. Growth factor-expressing human neural progenitor cell grafts protect motor neurons but do not ameliorate motor performance and survival in ALS mice Exp. Mol Med. 41, 487-500 (2009)
10. Ko, C.-P. & Robitaille, R. Perisynaptic Schwann Cells at the Neuromuscular Synapse: Adaptable. Multitasking Glial Cells. Cold Spring Harb. Perspect. Biol. 7: a020503 (2015)
11. Griffin, J. W. & Thompson, W. J. Biology and pathology of nonmyelinating schwann cells. Glia 56, 1518-1531 (2008)
12. Alvarez-Suarez, P., Gawor, M. & Prószyński, T. J. Pensynaptic schwann cells—The multitasking cells at the developing neuromuscular junctions. Semin. Cell Dev. Biol. 0-1 (2020) doi:10.1016/j.semcdb.2020.02.011.
13. Winter, F. De et al. The expression of the chemorepellent Semaphorin 3A is selectively induced in terminal Schwann cells of a subset of neuromuscular synapses that display limited anatomical plasticity and enhanced vulnerability in motor neuron disease. Mol. Cell. Neurosci. 32, 102-117 (2006).
14. Arbour, D., Tremblay, E., Martineau, E., Julien, J.-P. & Robitaille, R. Early and persistent abnormal decoding by glial cells at the neuromuscular junction in an ALS model. J. Neurosci. 35, 688-706 (2015).
15. Robitaille. R. Purinergic receptors and their activation by endogenous purines at pensynaptic glial cells of the frog neuromuscular junction. J. Neurosci. 15, 7121-31 (1995).
16. Rochon, D., Rousse, I. & Robitaille, R. Synapse-glia interactions at the mammalian neuromuscular junction. J. Neurosci. 21, 3819-29 (2001).
17. Robitaille, R. Modulation of synaptic efficacy and synaptic depression by glial cells at the frog neuromuscular junction, Neuron 21, 847-65 (1998).
18. Darabid, H, Arbour, D. & Robitaille, R Glial cells decipher synaptic competition at the mammalian neuromuscular junction. J. Neurosci. 33, 1297-1313 (2013).
19. VanSaun, M, Humburg, B. C., Arnett, M. G., Pence, M. & Werle, M. J. Activation of Matrix Metalloproteinase-3: s altered at the frog neuromuscular junction following changes in synaptic activity. Dev. Neurobiol. 67, 1486-97 (2007)
20. VanSaun, M. & Warle, M. J. Matrix metalloproteinase-3 removes agrin from synaptic basal lamina. J. Neurobiol. 43, 140-9 (2000).
21. Smith, I. W., Mikesh, M., Lee, Y. II & Thompson, W. J. Terminal Schwann cells participate in the competition underlying neuromuscular synapse elimination. J. Neurosci. 33, 17724-17736 (2013)
22. Duregotti. E. et al. Mitochondrial alarmins released by degenerating motor axon terminals activate perisynaptic Schwann cells. Proc. Natl. Acad. Sci. U.S.A. 112, E497-505 (2015)
23. Kawabuchi, M. et al. The spatiotemporal relationship among Schwann cells, axons and postsynaptic acetylcholine receptor regions during muscle reinnervation in aged rats. Anat. Rec. 264, 183-202 (2001).
24. Kang. H. & Lichtman, J. W. Motor axon regeneration and muscle reinnervation in young adult and aged animals. J. Neurosci. 33, 19480-19491 (2013).
25. Reddy, L. V., Koirala, S., Sugiura, Y, Herrera, A. A & Ko, C. P. Glial cells maintain synaptic structure and function and promote development of the neuromuscular junction in vivo. Neuron 40, 563-580 (2003).
26. Barik, A., LI. L., Sathyamurthy, A., Xiong, W. C. & Mei, L. Schwann cells in neuromuscular junction formation and maintenance. J. Neurosci. 36, 9770-9751 (2016).
27. Messing. A. et al. P0 promoter directs expression of reporter and toxin genes to schwann cells of transgenic mice. Neuron 8, 507-520 (1992).
28. Halstead, S. K. et al. Anti-disialoside antibodies kill perisynaptic Schwann cells and damage motor nerve terminals via membrane attack complex in a murine model of neuropathy. Brain 127, 2109-23 (2004).
29. Riethmacher, D. at al. Severe neuropathies in mice with targeted mutations in the ErbB3 receptor. Nature 389, 725-730 (1997)
30. Woldeyesus, M. T. et al. Peripheral nervous system defects in erb82 mutants following genetic rescue of heart development. Genes Dev. 13, 2538-2548 (1999).
31. Morris. J. K. et al. Rescue of the cardiac defect in ErbB2 mutant mice reveals essential roles of ErbB2 in peripheral nervous system development. Neuron 23, 273-83 (1999)
32. Lin. W. et al. Aberrant development of motor axons and neuromuscular synapses in erbB2-deficient mice. Proc. Natl. Acad. Sci. U.S.A. 97, 1299-1304 (2000).
33. Brockes, J. P., Fields. K. L. & Raff, M. C. Studies on cultured rat Schwann cells. I. Establishment of purified populations from cultures of peripheral nerve. Brain Res. 165, 105-118 (1979)
34. Perez. V. J. & Moore, B. W. WALLERIAN DEGENERATION IN RABBIT TIBIAL NERVE: CHANGES IN AMOUNTS OF THE S.100 PROTEIN. J. Neurochem 15, 971-977 (1968)
35. Stallcup, W. B. The NG2 antigen, a putative lineage marker: immunofluorescent localization in primary cultures of rat brain. Dev. Biol. 83, 154-66 (1981).
36. Bergles, D. E., Jabs, R. & Steinhäuser. C. Neuron-glia synapses in the brain. Brain Research Reviews vol. 63 130-137 (2010)
37. Zhu. X., Bergles, D. E. & Nishiyama, A. NG2 cells generale both oligodendrocytes and gray matter astrocyles. Development 135, 145-157 (2008).
38. Zuo. Y. et al. Fluorescent proteins expressed in mouse transgenic lines mark subsets of glia, neurons, macrophages, and dendritic cells for vital examination. J. Neurosci. 24, 10999-11009 (2004).
39. Girard, E. et al. Butyrylcholinesterase and the control of synaptic responses in acetylcholinesterase knockout mice. Life Sci. 80, 2380-2385 (2007).
40. Petrov. K. A. et al. Schwann cells sense and control acetylcholine spillover at the neuromuscular junction by a7 nicotinic receptors and butyrylcholinesterase. J. Neurosci. 34, 11870-83 (2014)
41. Kessaris, N. et al. Competing waves of oligodendrocytes in the forebrain and postnatal elimination of an embryonic lineage. Nat. Neurosci. 9, 173-9 (2006)
42. Zhu, X. et al. Age-dependent fate and lineage restriction of single NG2 cells. Development 138, 745-753 (2011)
43. Valdez, G., Tapia, J. C., Lichtman, J. W., Fox. M. A. & Sanes, J. R. Shared resistance to aging and ALS in neuromuscular junctions of specific muscles. PLOS One 7, e34640 (2012).
44. Li, Y., Lee. Y. il & Thompson, W. J. Changes in aging mouse neuromuscular junctions are explained by degeneration and regeneration of muscle fiber segments at the synapse. J. Neurosci. 31, 14910-9 (2011).
45. Carrasco, D. L., Sebum. K. L & Pinter, M J. Altered terminal Schwann cell morphology precedes denervation in SOD1 mice. Exp. Neurol. 275, 172-181 (2016).
46. Ludatscher. R. M., Silbermann. M., Gershon, D. & Reznick, A. Evidence of schwann cell degeneration in the aging mouse motor end-plate region. Exp. Gerontol. 20, 81-91 (1985).
47. Wokke, J. H. et al. Morphological changes in the human end plate with age. J. Neurol. Sci. 95, 291-310 (1990).
48. Brill. M. S., Lichtman, J. W., Thompson, W., Zuo, Y. & Misgeld, T. Spatial constraints dictate glial territories at murine neuromuscular junctions. J. Cell Biol. 195, 293-305 (2011)
49. Wassle, H. & Riemann, H. J. The mosaic of nerve cells in the mammalian retina. Proc. R. Soc. London—Biol. Sci. (1978) doi: 10.1098/rspb. 1978.0026.
50. Cook, J. E. Spatial properties of retinal mosaics: An empirical evaluation of some existing measures. Vis. Neurosci. (1996) doi: 10.1017/80962523800007094.
51. Feng, G. et al. Imaging neuronal subsets in transgenic mice expressing multiple spectral variants of GFP. Neuron 28, 41-51 (2000).
52. Castro, R. et al. identification of a molecular fingerprint for synaptic glia. bioRxiv 2020.03.13.991323 (2020) doi 10.1101/2020.03.13.991323.
53. Aronesty, E. Comparison of Sequencing Utility Programs. Open Bioinforma. J. 7, 1-8 (2013).
54. Kong. Y. Btrim: A fast, lightweight adapter and quality trimming program for next-generation sequencing technologies. Genomics 98, 162-153 (2011)
55. Kim, D., Langmead, B. & Salzberg, S. L. HISAT: a fast spliced aligner with low memory requirements. Nat. Methods 12, 357-60 (2015),
56. Love. M. L., Huber, W. & Anders, S. Moderated estimation of fold change and dispersion for RNA-seq data with DESeq2. Genome Biol. 15, 550 (2014).
57. Maxwell. N. et al. α-Motor neurons are spared from aging while their synaptic inputs degenerate in monkeys and mice. Aging Cell 17, e12726 (2018)
58. Valdez, G. et al. Attenuation of age-related changes in mouse neuromuscular synapses by caloric restriction and exercise. Proc. Natl. Acad. Sci. U.S.A. 107, 14863-8 (2010)
59. Terauchi. A. et al. Distinct FGFs promote differentiation of excitatory and inhibitory synapses. Nature 465, 783-7 (2010)
60. Georgiou, J. & Chariton, M. P. Non-myelin-forming perisynaptic Schwann cells express protein zero and myelin-associated glycoprotein. Glia 27, 101-109 (1999).
61. Georgiou, J., Robitaille, R., Trimble. W. S & Chariton, M. P. Synaptic regulation of glial protein expression in vivo. Neuron 12, 443-455 (1994)
62. Wright, M. C. et al. Distinct Muscarinic Acetylcholine Receptor Subtypes Contribute to Stability and Growth, But Not Compensatory Plasticity, of Neuromuscular Synapses. J. Neurosci. 29, 14942-14955 (2009).
63. Darabid, H., St-Pierre-See. A. & Robitaille, R. Purinergic-Dependent Glial Regulation of Synaptic Plasticity of Competing Terminals and Synapse Elimination at the Neuromuscular Junction. Cell Rep. 25, 2070-2082.66 (2018).
64. Beaino, W. et al. Purinergic receptors P2Y12R and P2X7R: potential targets for PET imaging of microglia phenotypes in multiple sclerosis. J. Neuroinflammation 14, 259 (2017)
65. Jacobson, K. A. et al. Update of P2Y Receptor Pharmacology: IUPHAR Review: 27. Br. J. Pharmacol. (2020) doi: 10.1111/bph.15005.
66. Reichert, F., Saada. A. & Rotshenker. S Peripheral nerve injury induces Schwann cells to express two macrophage phenotypes: Phagocytosis and the galactose-specific lectin MAC-2. J. Neurosci. 14, 3231-3245 (1994).
67. Tremblay, E., Martineau, E & Robitaille, R. Opposite synaptic alterations at the neuromuscular junction in an ALS mouse model: When motor units matter J. Neurosci. 37, 8901-6918 (2017)
68. Haynes. S. E. et al. The P2Y12 receptor regulates microglial activation by extracellular nucleotides. Nat. Neurosci. 9, 1512-1519 (2006).

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize or be able to ascertain using no more than routine experimentation, many equivalents to the specific embodiments described herein. Such equivalents are intended to be encompassed by the following exemplary claims.

We claim:

1. A method of treating a neuromuscular condition or a nervous condition in a subject in need thereof, the method comprising the steps of:
(1) obtaining a subject diagnosed with a condition, suspected of having the condition, or a subject with a likelihood of developing the condition; and
(2) administering a therapeutically effective amount of one or more of Clopidogrel, Ticagrelor, Prasugrel, and/or Cangrelor, or a therapeutically effective amount of a salt of one or more of Clopidogrel, Ticagrelor, Prasugrel, and/or Cangrelor thereof, a prodrug of one or more of Clopidogrel, Ticagrelor, Prasugrel, and/or Cangrelor thereof, a hydrate and/or a solvate of one or more of Clopidogrel, Ticagrelor, Prasugrel, and/or Cangrelor thereof, or a combination thereof; 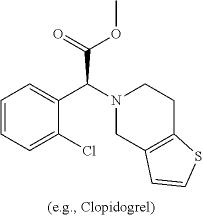 [SMILES: COC(=O)[C@H](c1ccccc1Cl)N1CCc2sccc2C1]
whereby neuromuscular junctions (NMJs) are preserved and repaired in the subject by the administering.

2. The method of claim 1, wherein the neuromuscular condition is selected from the group consisting of amyotrophic lateral sclerosis (ALS), spinal muscular atrophy (SMA), Duchenne muscular dystrophy, Becker muscular dystrophy, myasthenia gravis, Charcot-Marie-Tooth disease, spinal cord injury, a condition due to the normal aging process, sarcopenia, cachexia, peripheral demyelination diseases, nerve damages, nerve injuries, damages due to strenuous physical activities, fatigue, aging diseases, or other conditions that affect synapses and/or skeletal muscles.

3. The method of claim 1, wherein the nervous condition is selected from the group consisting of Alzheimer's disease, Parkinson's disease, Huntington's disease, multiple sclerosis, amyotrophic lateral sclerosis (ALS), spinal muscular atrophy (SMA), Charcot-Marie-Tooth disease, Friedreich's ataxia, Lewy body dementia, vascular dementia, frontotemporal dementia, progressive supranuclear palsy, corticobasal degeneration, chronic traumatic encephalopathy, Creutzfeldt-Jakob disease, and/or neurodegenerative disorders.

4. The method of claim 1, wherein the administering is performed via a route selected from the group consisting of oral, parenteral, subcutaneous, intravenous, intramuscular, intraperitoneal, transdermal, intranasal, and rectal administration.

5. The method of claim 1, wherein the one or more of Clopidogrel, Ticagrelor, Prasugrel, and/or Cangrelor or its pharmaceutically acceptable forms is administered in a dosage form optimized for the selected route of administration, the dosage form being selected from the group consisting of a tablet, a capsule, a solution, a suspension, an emulsion, a powder, a granule, a suppository, an injection, an infusion, and an implant.

6. The method of claim 1, wherein the therapeutically effective amount of one or more of Clopidogrel, Ticagrelor, Prasugrel, and/or Cangrelor is determined based on the severity and progression of the neuromuscular or nervous condition, an age, weight, and general health of the subject, and an optimal balance between therapeutic efficacy and potential side effects, and ranges from about 1 mg to about 1000 mg per day.

7. The method of claim 1, wherein the therapeutically effective amount of one or more of Clopidogrel, Ticagrelor, Prasugrel, and/or Cangrelor is administered as a single daily dose or divided into multiple doses per day to maintain a consistent plasma concentration and to minimize potential adverse effects while maximizing therapeutic benefits.

8. The method of claim 1, wherein the therapeutically effective amount of one or more of Clopidogrel, Ticagrelor, Prasugrel, and/or Cangrelor is administered in a designed dosing regimen of once, twice, or thrice daily, taking into account the pharmacokinetic and pharmacodynamic properties of one or more of Clopidogrel, Ticagrelor, Prasugrel, and/or Cangrelor, to ensure optimal receptor occupancy and to minimize the risk of tachyphylaxis or tolerance development.

9. The method of claim 1, wherein the method modulates P2Y12 receptors in perisynaptic Schwann cells in the subject.

10. The method of claim 1, wherein the method is executed in combination with a physical therapy, a gene therapy, a stem cell therapy, administration with other drugs, or a combination thereof.

11. A method of preserving and repairing damages to neuromuscular junctions (NMJs) in a subject having, suspected of having, or likely to develop a neuromuscular condition or a nervous condition, the method comprising:
administering to the subject a therapeutically effective amount of one or more of Clopidogrel, Ticagrelor, Prasugrel, and/or Cangrelor and/or its pharmaceutically acceptable forms.

12. The method of claim 11, wherein the subject is diagnosed with the neuromuscular condition or the nervous condition, is suspected of having the neuromuscular condition or the nervous condition, or has a likelihood of developing the neuromuscular condition or the nervous condition.

13. The method of claim 11, wherein the pharmaceutically acceptable forms of one or more of Clopidogrel, Ticagrelor, Prasugrel, and/or Cangrelor comprise one or more of a salt of one or more of Clopidogrel, Ticagrelor, Prasugrel, and/or Cangrelor, a prodrug of one or more of Clopidogrel, Ticagrelor, Prasugrel, and/or Cangrelor, a hydrate of one or more of Clopidogrel, Ticagrelor, Prasugrel, and/or Cangrelor, a solvate of one or more of Clopidogrel, Ticagrelor, Prasugrel, and/or Cangrelor, or a combination thereof.

14. The method of claim 11, wherein the neuromuscular condition is selected from the group consisting of amyotrophic lateral sclerosis (ALS), spinal muscular atrophy (SMA), Duchenne muscular dystrophy, Becker muscular dystrophy, myasthenia gravis, Charcot-Marie-Tooth disease, spinal cord injury, a condition due to the normal aging process, nerve damages, nerve injuries, damages due to strenuous physical activities, fatigue, or other conditions that affect synapses as well as skeletal muscles.

15. The method of claim 11, wherein the nervous condition is selected from the group consisting of Alzheimer's disease, Parkinson's disease, Huntington's disease, multiple sclerosis, amyotrophic lateral sclerosis (ALS), spinal muscular atrophy (SMA), Charcot-Marie-Tooth disease, Friedreich's ataxia, Lewy body dementia, vascular dementia, frontotemporal dementia, progressive supranuclear palsy, corticobasal degeneration, chronic traumatic encephalopathy, Creutzfeldt-Jakob disease, and other neurodegenerative disorders.

16. The method of claim 11, wherein the effective amount of one or more of Clopidogrel, Ticagrelor, Prasugrel, and/or Cangrelor or its pharmaceutically acceptable forms is a therapeutically effective amount that is determined based on a comprehensive assessment of the subject's medical history, the severity and progression of the neuromuscular or nervous condition, the presence of comorbidities, the concomitant use of other medications, and the individual pharmacokinetic and pharmacodynamic profile of the subject, and is adjusted as necessary to achieve the optimal balance between therapeutic efficacy and safety, while minimizing the risk of adverse effects and drug interactions, and is administered in a personalized dosing regimen that takes into account the subject's age, weight, gender, and other relevant factors, and is monitored and modified as needed to ensure the best possible outcomes for the subject.

17. The method of claim 11, wherein the administering is performed via a route selected from the group consisting of oral, parenteral, subcutaneous, intravenous, intramuscular, intraperitoneal, transdermal, intranasal, and rectal administration.

18. The method of claim 11, wherein the one or more of Clopidogrel, Ticagrelor, Prasugrel, and/or Cangrelor or its pharmaceutically acceptable forms is administered in a dosage form optimized for the selected route of administration, the dosage form being selected from the group consisting of a tablet, a capsule, a solution, a suspension, an emulsion, a powder, a granule, a suppository, an injection, an infusion, and an implant.
